(19) United States
(12) Patent Application Publication
(10) Pub. No.: US 2025/0351662 A1
(43) Pub. Date: Nov. 13, 2025

(54) ORGANIC COMPOUND AND ORGANIC LIGHT EMITTING DEVICE COMPRISING SAME

(71) Applicant: LG CHEM, LTD., Seoul (KR)

(72) Inventors: Jae Seung Ha, Daejeon (KR); Sujeong Geum, Daejeon (KR); Woochul Lee, Daejeon (KR); Jiye Jeon, Daejeon (KR); Woo Jin Cho, Daejeon (KR); Ji Young Choi, Daejeon (KR)

(21) Appl. No.: 19/093,501

(22) Filed: Mar. 28, 2025

(30) Foreign Application Priority Data
May 10, 2024 (KR) 10-2024-0061689

(51) Int. Cl. H10K 50/12; H10K 85/60; H10K 101/00

(52) U.S. Cl. CPC H10K 50/12; H10K 85/622; H10K 85/626; H10K 85/633; H10K 85/636; H10K 85/654; H10K 85/655; H10K 85/6572; H10K 85/6574; H10K 85/6576; H10K 85/658; H10K 2101/90

(57) ABSTRACT

An organic compound of Chemical Formula 1:

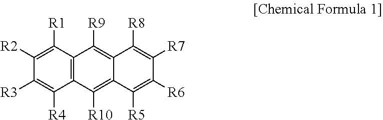

wherein: one or more of R1 to R10 are bonded to L1 in Chemical Formula 1-A below; one or more of R1 to R10 not bonded to L1 in Chemical Formula 1-A above are bonded to L2 in Chemical Formula 1-B below:

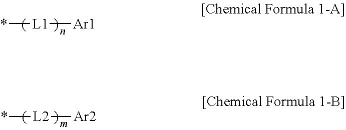

Ar1 is Chemical Formula 1-C, and Ar2 is Chemical Formula 1-D:

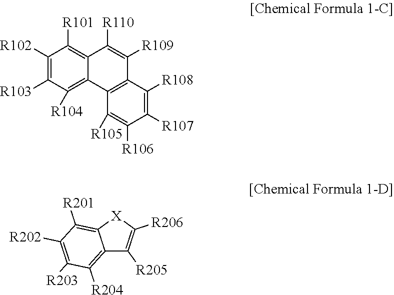

X is O or S; one of R101 to R108 is bonded to L1; at least one adjacent pair in R201 to R206 is bonded to each other to form one or more substituted or unsubstituted ring groups; any one of R201 to R206 not forming the substituted or unsubstituted ring group is bonded to L2; and the other substituents are as defined in the specification; and an organic light emitting device including the same.

### CROSS-REFERENCE TO RELATED APPLICATIONS

This application claims priority to and the benefit of Korean Patent Application No. 10-2024-0061689 filed in the Korean Intellectual Property Office on May 10, 2024, the entire contents of which are incorporated herein by reference.

### TECHNICAL FIELD

The present specification relates to an organic compound and an organic light emitting device including the same.

### BACKGROUND ART

An organic light emission phenomenon generally refers to a phenomenon converting electrical energy to light energy using an organic material. An organic light emitting device using the organic light emitting phenomenon usually has a structure including an anode, a cathode, and an organic material layer interposed therebetween. Here, the organic material layer has in many cases a multi-layered structure composed of different materials in order to improve the efficiency and stability of the organic light emitting device, and for example, may be composed of a hole injection layer, a hole transport layer, a light emitting layer, an electron transport layer, an electron injection layer, and the like. In the structure of the organic light emitting device, if a voltage is applied between two electrodes, holes are injected from an anode into the organic material layer and electrons are injected from a cathode into the organic material layer, and when the injected holes and electrons meet each other, an exciton is formed, and light is emitted when the exciton falls down again to a ground state.

There is a continuous need for developing a new material for the aforementioned organic light emitting device.

### RELATED ART DOCUMENTS

### Patent Document

(Patent Document 1) Chinese Patent Application Publication No. 114621274

### SUMMARY OF THE INVENTION

The present invention has been made in an effort to provide an organic compound and an organic light emitting device including the same.

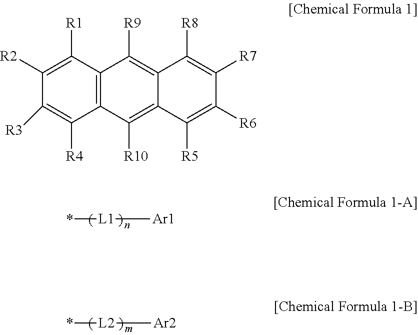

In Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C,

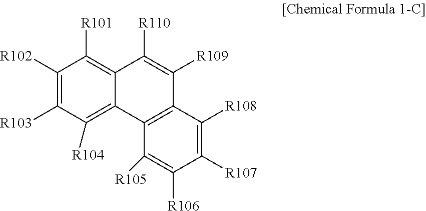

in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is necessarily bonded to L1,
Ar2 is the following Chemical Formula 1-D,

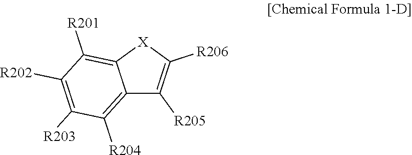

in Chemical Formula 1-D,
X is O or S,
R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups,
the substituted or unsubstituted ring group; or any one of R201 to R206 not forming the substituted or unsubstituted ring group is bonded to L2, and
* is a moiety bonded to Chemical Formula 1.

An exemplary embodiment of the present invention provides an organic light emitting device including: an anode; a cathode; and an organic material layer having one or more layers provided between the anode and the cathode, in which one or more layers of the organic material layers include the above-described organic compound.

The organic compound described in the present specification can be used as a material for an organic material layer of an organic light emitting device.

The organic compound according to at least one exemplary embodiment of the present specification may improve the efficiency, achieve low driving voltage and/or improve service life characteristics in the organic light emitting device.

### BRIEF DESCRIPTION OF THE DRAWINGS

FIG. 1 exemplifies a structure of an organic light emitting device in which a first electrode 2, a light emitting layer 4, and a second electrode 3 are sequentially stacked on a substrate 1. The compound is included in the light emitting layer.

FIG. 2 exemplifies the structure of an organic light emitting device in which a first electrode 2, a hole injection layer 5, a hole transport layer 6, a hole adjusting layer 7, a light emitting layer 4, an electron adjusting layer 8, an electron transport layer 9, an electron injection layer 10, a second electrode 3, and a capping layer 11 are sequentially stacked on a substrate 1. The compound is included in the light emitting layer.

FIG. 3 is an MS graph of Compound A.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

In the present specification, “dotted line (---)” means a position bonded to a chemical formula or a compound.

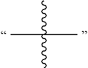

In the present specification, “s” means a position bonded to a formula or a compound.

In the present specification, “energy level” means a size of energy. Therefore, the energy level is interpreted to mean the absolute value of the corresponding energy value. For example, a low or deep energy level means that the absolute value increases in the negative direction from the vacuum level.

In the present specification, the highest occupied molecular orbital (HOMO) means a molecular orbital (highest occupied molecular orbital) in the highest energy region in regions in which electrons can participate in bonding, the lowest unoccupied molecular orbital (LUMO) means the molecular orbital (lowest unoccupied molecular orbital) in which electrons are present in the lowest energy region among the semi-bonded regions, and the HOMO energy level means the distance from the vacuum level to the HOMO. Furthermore, the LUMO energy level means the distance from the vacuum level to the LUMO.

In the present specification, a bandgap means a difference in energy level between HOMO and LUMO, that is, a HOMO-LUMO gap (Gap).

In the present specification, the HOMO energy level may be measured using a photoelectron spectrometer under the atmosphere (manufactured by RIKEN KEIKI Co., Ltd.: AC3), and the LUMO energy level may be calculated from wavelength values measured through photoluminescence (PL).

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present invention, the term “substituted or unsubstituted” means being substituted with one or two or more substituents selected from the group consisting of deuterium; a halogen group; a cyano group (—CN); a nitro group; a hydroxyl group; an alkyl group; a cycloalkyl group; an alkoxy group; a phosphine oxide group; an aryloxy group; an alkylthioxy group; an arylthioxy group; an alkylsulfoxy group; an arylsulfoxy group; an alkenyl group; a silyl group; a boron group; an amine group; an aryl group; or a heterocyclic group, being substituted with a substituent to which two or more substituents among the exemplified substituents are linked, or having no substituent. For example, “the substituent to which two or more substituents are linked” may be a biphenyl group. That is, the biphenyl group may also be an aryl group, and may be interpreted as a substituent to which two phenyl groups are linked.

In the present specification, the term “substituted or unsubstituted” means being substituted with one or two or more substituents selected from the group consisting of deuterium; a halogen group; a cyano group; a silyl group; an alkoxy group; an aryloxy group; an alkyl group; an aryl group; and a heterocyclic group, being substituted with a substituent to which two or more substituents among the exemplified substituents are linked, or having no substituent.

In the present specification, the term “substituted or unsubstituted” means being substituted with one or two or more substituents selected from the group consisting of deuterium; an alkyl group; an aryl group; and a heterocyclic group, being substituted with a substituent to which two or more substituents among the exemplified substituents are linked, or having no substituent.

In the present specification, the fact that two or more substituents are linked indicates that hydrogen of any one substituent is linked to another substituent. For example, an isopropyl group and a phenyl group may be linked to each other to become a substituent of

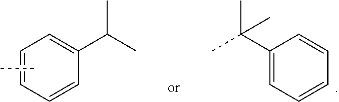

In the present specification, the fact that three substituents are linked to one another includes not only a case where (Substituent 1)-(Substituent 2)-(Substituent 3) are consecutively linked to one another, but also a case where (Substituent 2) and (Substituent 3) are linked to (Substituent 1). For example, two phenyl groups and an isopropyl group may be linked to each other to become a substituent of

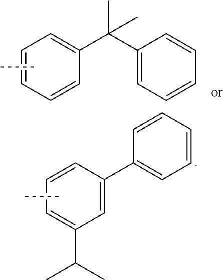

The same also applies to the case where four or more substituents are linked to one another.

Examples of the substituents will be described below; however, the substituents are not limited thereto.

In the present specification, examples of a halogen group include fluorine (—F), chlorine (—Cl), bromine (—Br) or iodine (—I).

In the present specification, a silyl group may be represented by a formula of —SiYaYbYc, and Ya, Yb, and Yc may be each hydrogen; a substituted or unsubstituted alkyl group; or a substituted or unsubstituted aryl group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be represented by a formula of —BYdYe, and Yd and Ye may be each hydrogen; a substituted or unsubstituted alkyl group; or a substituted or unsubstituted aryl group. Specific examples of the boron group include a trimethylboron group, a triethylboron group, a t-butyldimethylboron group, a triphenylboron group, a phenylboron group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the above-described description on the alkyl group may be applied to an arylalkyl group, except that the arylalkyl group is substituted with an aryl group.

In the present specification, the alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, an alkenyl group may be straight-chained or branched, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10. According to still another exemplary embodiment, the number of carbon atoms of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, the alkynyl group may be straight-chained or branched as a substituent including a triple bond between a carbon atom and a carbon atom, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to another embodiment, the number of carbon atoms of the cycloalkyl group is from 3 to 6. Specific examples thereof may include a cyclopropyl group, a cyclobutyl group, a cyclopentyl group, a cyclohexyl group, a cycloheptyl group, a cyclooctyl group and the like, but are not limited thereto.

In the present specification, an amine group is —NH₂, and the amine group may be substituted with the above-described alkyl group, aryl group, heterocyclic group, alkenyl group, cycloalkyl group, a combination thereof, and the like. The number of carbon atoms of the substituted amine group is not particularly limited, but is preferably 1 to 30. According to an exemplary embodiment, the number of carbon atoms of the amine group is 1 to 20. According to an exemplary embodiment, the number of carbon atoms of the amine group is 1 to 10. Specific examples of the substituted amine group include a methylamine group, a dimethylamine group, an ethylamine group, a diethylamine group, a phenylamine group, a 9,9-dimethylfluorenylphenylamine group, a pyridylphenylamine group, a diphenylamine group, a phenylpyridylamine group, a naphthylamine group, a biphenylamine group, an anthracenylamine group, a dibenzofuranylphenylamine group, a 9-methylanthracenylamine group, a diphenylamine group, a phenylnaphthylamine group, a ditolylamine group, a phenyltolylamine group, a diphenylamine group, and the like, but are not limited thereto.

In the present specification, an aryl group is not particularly limited, but has preferably 6 to 60 carbon atoms, and may be a monocyclic aryl group or a polycyclic aryl group. According to an exemplary embodiment, the number of carbon atoms of the aryl group is from 6 to 30. According to an exemplary embodiment, the number of carbon atoms of the aryl group is from 6 to 20. The aryl group may be an aryl group composed of a single ring or a polycyclic aryl group (a bicyclic or more aryl group). The aryl group composed of the single ring may mean a phenyl group; or a group to which two or more phenyl groups are linked. Examples of the aryl group composed of the sing ring include a phenyl group, a biphenyl group, a terphenyl group, a quaterphenyl group, and the like, but are not limited thereto. The polycyclic aryl group may mean a group in which two or more monocyclic rings such as a naphthyl group and a phenanthrenyl group are fused. Examples of the polycyclic aryl group include a naphthyl group, an anthracenyl group, a phenanthrenyl group, a pyrenyl group, a perylenyl group, a chrysenyl group, a fluorenyl group, a triphenylenyl group, and the like, but are not limited thereto.

In the present specification, a fluorenyl group may be substituted, and two substituents may be bonded to each other to form a spiro structure.

When the fluorenyl group is substituted, the substituent may be a spirofluorenyl group such as

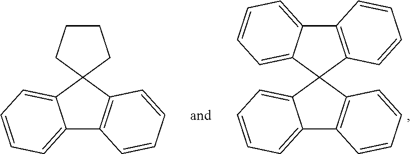

and/and a substituted fluorenyl group such as

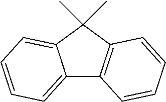

(a 9,9-dimethylfluorenyl group) and

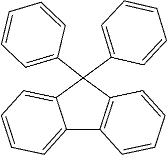 [SMILES: c1ccc(C2(c3ccccc3)c3ccccc3-c3ccccc32)cc1]

(a 9,9-diphenylfluorenyl group). However, the substituent is not limited thereto.

In the present specification, the above-described description on the aryl group may be applied to an aryl group in an aryloxy group.

In the present specification, a heterocyclic group is a cyclic group including one or more of N, O, P, S, Si, and Se as a heteroatom, and the number of carbon atoms thereof is not particularly limited, but is preferably 2 to 60. According to one embodiment, the number of carbon atoms of the heterocyclic group is from 2 to 30. According to one embodiment, the number of carbon atoms of the heterocyclic group is from 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzonaphthothiophene group, an indenocarbazole group, a triazinyl group, and the like, but are not limited thereto.

In the present specification, the above-described description on the heterocyclic group may be applied to a heteroaryl group except for an aromatic heteroaryl group.

In the present specification, a heteroaryl group includes one or more atoms other than carbon, that is, one or more heteroatoms, and specifically, the heteroatom may include one or more atoms selected from the group consisting of O, N, Se, S, and the like. The number of carbon atoms thereof is not particularly limited, but is preferably 2 to 30, and the heteroaryl group may be monocyclic or polycyclic. Examples of the heteroaryl group include a thiophene group, a furan group, a pyrrole group, an imidazole group, a thiazole group, an oxazole group, an oxadiazole group, a pyridine group, a bipyridine group, a pyrimidine group, a triazine group, a triazole group, an acridine group, a pyridazine group, a pyrazine group, a quinoline group, a quinazoline group, a quinoxaline group, a phthalazine group, a pyridopyrimidine group, a pyridopyrazine group, a pyrazinopyrazine group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthridine group, a phenanthroline group, an isoxazole group, a thiadiazole group, a dibenzofuran group, a dibenzosilole group, a phenoxathiine group, a phenoxazine group, a phenothiazine group, a dihydroindenocarbazole group, a spirofluorenexanthene group, a spirofluorenethioxanthene group, and the like, but are not limited thereto.

In the present specification, the description on the aryl group may be applied to an arylene group except for a divalent arylene group.

In the present specification, the description on the heterocyclic group may be applied to a divalent heterocyclic group except for a divalent heterocyclic group.

In the present specification, the description on the aryl group may be applied to a (n+1)-valent aryl group except for a (n+1)-valent aryl group.

In the present specification, the description on the heterocyclic group may be applied to a (n+1)-valent heterocyclic group except for a (n+1)-valent heterocyclic group.

In the present specification, in a substituted or unsubstituted ring formed by being bonded to an adjacent group, the “ring” means a hydrocarbon ring; or a hetero ring.

The hydrocarbon ring may be an aromatic ring, an aliphatic ring, or a fused ring of the aromatic ring and the aliphatic ring, and may be selected from the examples of the cycloalkyl group or the aryl group.

In the present specification, being bonded to an adjacent group to form a ring means being bonded to an adjacent group to form a substituted or unsubstituted aliphatic hydrocarbon ring; a substituted or unsubstituted aromatic hydrocarbon ring; a substituted or unsubstituted aliphatic hetero ring; a substituted or unsubstituted aromatic hetero ring; or a fused ring thereof. The hydrocarbon ring means a ring composed only of carbon and hydrogen atoms. The hetero ring means a ring including one or more selected from among N, O, P, S, Si and Se. In the present specification, the aliphatic hydrocarbon ring, the aromatic hydrocarbon ring, the aliphatic hetero ring, and the aromatic hetero ring may be monocyclic or polycyclic.

In the present specification, the aliphatic hydrocarbon ring means a ring composed only of carbon and hydrogen atoms as a non-aromatic ring. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, and the like, but are not limited thereto.

In the present specification, an aromatic hydrocarbon ring means an aromatic ring composed only of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, and the like, but are not limited thereto. In the present specification, the aromatic hydrocarbon ring may be interpreted to have the same meaning as the aryl group.

In the present specification, an aliphatic hetero ring means an aliphatic ring including one or more of hetero atoms. Examples of the aliphatic hetero ring include oxirane, tetrahydrofuran, 1,4-dioxane, pyrrolidine, piperidine, morpholine, oxepane, azocane, thiocane, and the like, but are not limited thereto.

In the present specification, an aromatic hetero ring means an aromatic ring including one or more of hetero atoms. Examples of the aromatic hetero ring include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxine, triazine, tetrazine, isoquinoline, quinoline, quinone, quinazoline, quinoxaline, naphthyridine, acridine, phenanthridine, diaza naphthalene, triazaindene, indole, indolizine, benzothiazole, benzoxazole, benzoimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, and the like, but are not limited thereto.

In the present specification, a fused ring means a cyclic structure in a form where two or more rings share two or more atoms. The fused ring may be an aromatic hydrocarbon ring, an aliphatic hydrocarbon ring, or a fused ring of an aromatic hydrocarbon ring and an aliphatic hydrocarbon ring, but is not limited thereto.

In the present specification, the fused aromatic hydrocarbon ring group means a ring in which two or more aromatic hydrocarbon rings are fused together. Examples of the fused aromatic hydrocarbon ring group may include a naphthyl group, an anthracenyl group, a phenanthrenyl group, a pyrenyl group, a perylenyl group, a triphenylene group, a chrysenyl group, a fluorenyl group, a triphenylenyl group, and the like, but are not limited thereto.

Unless otherwise defined in the present specification, all technical and scientific terms used in the present specification have the same meaning as commonly understood by one with ordinary skill in the art to which the present invention pertains. Although methods and materials similar to or equivalent to those described in the present specification may be used in the practice or in the test of exemplary embodiments of the present invention, suitable methods and materials will be described below. All publications, patent applications, patents, and other references mentioned in the present specification are hereby incorporated by reference in their entireties, and in the case of conflict, the present specification, including definitions, will control unless a particular passage is mentioned. Further, the materials, methods, and examples are illustrative only and are not intended to be limiting.

Hereinafter, preferred exemplary embodiments of the present invention will be described in detail. However, the exemplary embodiments of the present invention may be modified into various other forms, and the scope of the present invention is not limited to the exemplary embodiments which will be described below.

The organic light emitting device of the present invention is characterized by including both the compound represented by Chemical Formula 1 and the compound represented by Chemical Formula 2, and the organic light emitting device of the present invention exhibits the effects of low voltage, high efficiency and/or long service life.

Hereinafter, an organic compound of the following Chemical Formula 1 will be described in detail.

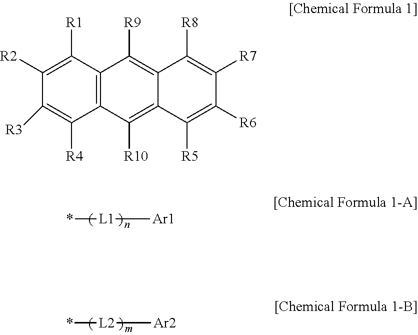

In Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C,

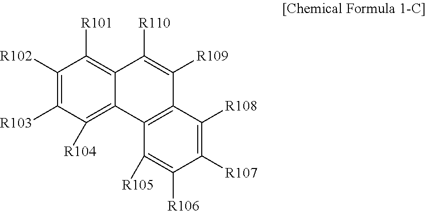

in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is necessarily bonded to L1,
Ar2 is the following Chemical Formula 1-D,

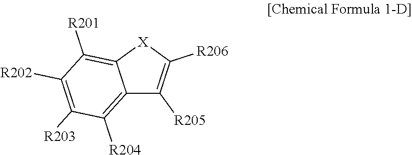

in Chemical Formula 1-D,
X is O or S,
R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups,
the substituted or unsubstituted ring group; or any one of R201 to R206 not forming the substituted or unsubstituted ring group is bonded to L2, and
* is a moiety bonded to Chemical Formula 1.

In an exemplary embodiment of the present specification, the substituted or unsubstituted ring group of Chemical Formula 1-D is an organic compound which is any one of the following Chemical Formulae 1-D-1 to 1-D-8.

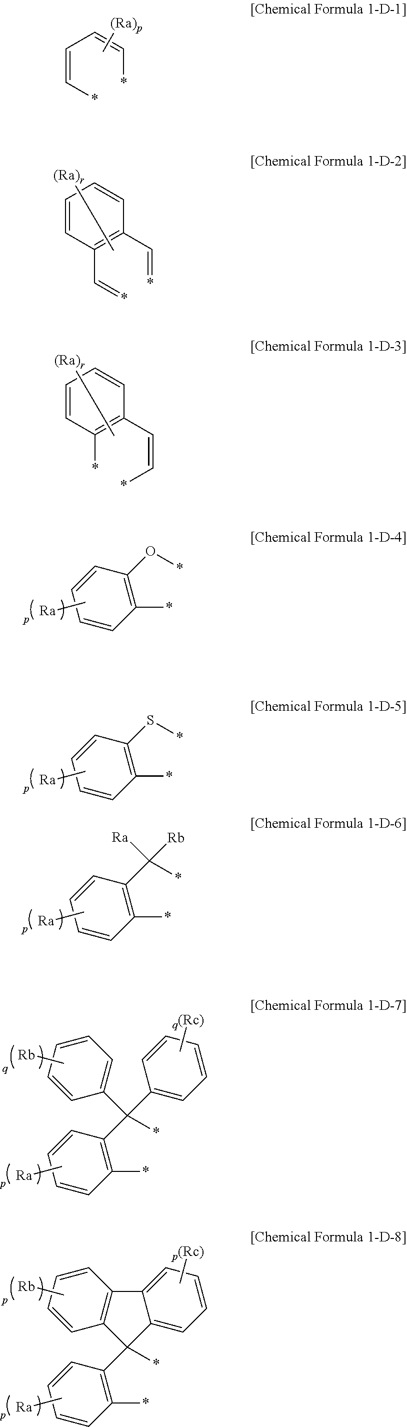

In Chemical Formulae 1-D-1 to 1-D-8,
Ra to Rc are the same as or different from each other, and are each independently hydrogen; deuterium; a substituted or unsubstituted alkyl group; a substituted or unsubstituted aryl group; or a substituted or unsubstituted heterocyclic group, or optionally bonded to an adjacent group to form a substituted or unsubstituted ring group, or bonded to L2 above,
p is an integer from 1 to 4, and when p is 2 or higher, two or more Ra's, Rb's, or Rc's are the same as or different from each other,
q is an integer from 1 to 5, and when q is 2 or higher, two or more Rb's or Rc's are the same as or different from each other,
r is an integer from 1 to 6, and when r is 2 or higher, two or more Ra's are the same as or different from each other, and
* is a moiety bonded to an adjacent pair of R201 to R206 in Chemical Formula 1-D.

In an exemplary embodiment of the present specification, Ra to Rc are the same as or different from each other, and are each independently hydrogen; deuterium; a substituted or unsubstituted alkyl group having 1 to 30 carbon atoms; a substituted or unsubstituted aryl group having 6 to 30 carbon atoms; or a substituted or unsubstituted heterocyclic group having 2 to 30 carbon atoms, or optionally bonded to an adjacent group to form a substituted or unsubstituted ring group, or bonded to L2.

In an exemplary embodiment of the present specification, Ra to Rc are the same as or different from each other, and are each independently hydrogen; deuterium; a substituted or unsubstituted alkyl group having 1 to 20 carbon atoms; a substituted or unsubstituted aryl group having 6 to 20 carbon atoms; or a substituted or unsubstituted heterocyclic group having 2 to 20 carbon atoms, or optionally bonded to an adjacent group to form a substituted or unsubstituted ring group, or bonded to L2.

In an exemplary embodiment of the present specification, Ra to Rc are the same as or different from each other, and are each independently hydrogen; deuterium; a substituted or unsubstituted alkyl group having 1 to 10 carbon atoms; a substituted or unsubstituted aryl group having 6 to 10 carbon atoms; or a substituted or unsubstituted heterocyclic group having 2 to 10 carbon atoms, or optionally bonded to an adjacent group to form a substituted or unsubstituted ring group, or bonded to L2.

In an exemplary embodiment of the present specification, Ra to Rc are the same as or different from each other, and are each independently hydrogen or deuterium, or optionally bonded to an adjacent group to form a substituted or unsubstituted ring group, or bonded to L2.

In an exemplary embodiment of the present specification, Ra to Rc are the same as or different from each other, and are each independently hydrogen, or optionally bonded to an adjacent group to form a substituted or unsubstituted ring group.

In an exemplary embodiment of the present specification, Ra to Rc are the same as or different from each other, and are each independently deuterium, or optionally bonded to an adjacent group to form a substituted or unsubstituted ring group, or bonded to L2.

An exemplary embodiment of the present specification is an organic compound in which Chemical Formula 1 above is any one of the following Chemical Formulae 1-1 to 1-4.

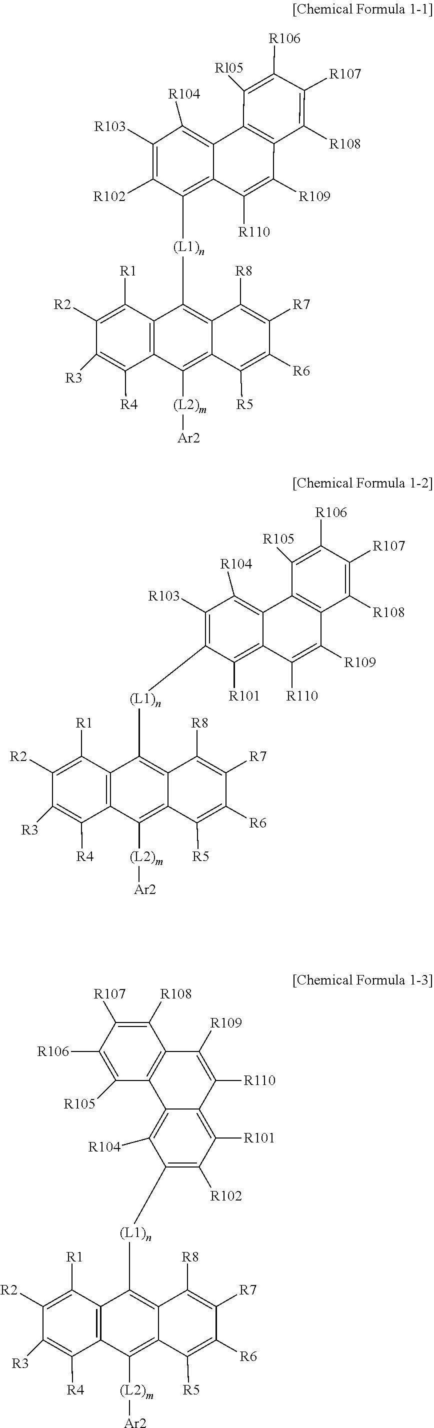

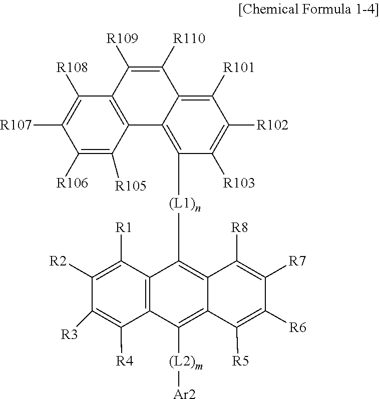

In Chemical Formulae 1-1 to 1-4,
the definitions of L1, L2, Ar2, n, and m are the same as the definitions in Chemical Formula 1 above,
R1 to R8 are each independently hydrogen; deuterium; a substituted or unsubstituted alkyl group; a substituted or unsubstituted cycloalkyl group; a substituted or unsubstituted aryl group; or a substituted or unsubstituted heteroaryl group, and
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group.

In the present specification, Chemical Formula 1 above is Chemical Formula 1-1 above.

In the present specification, Chemical Formula 1 above is Chemical Formula 1-2 above.

In the present specification, Chemical Formula 1 above is Chemical Formula 1-3 above.

In the present specification, Chemical Formula 1 above is Chemical Formula 1-4 above.

An exemplary embodiment of the present specification is an organic compound in which Chemical Formula 1 above is any one of the following Chemical Formulae 1-5 to 1-10.

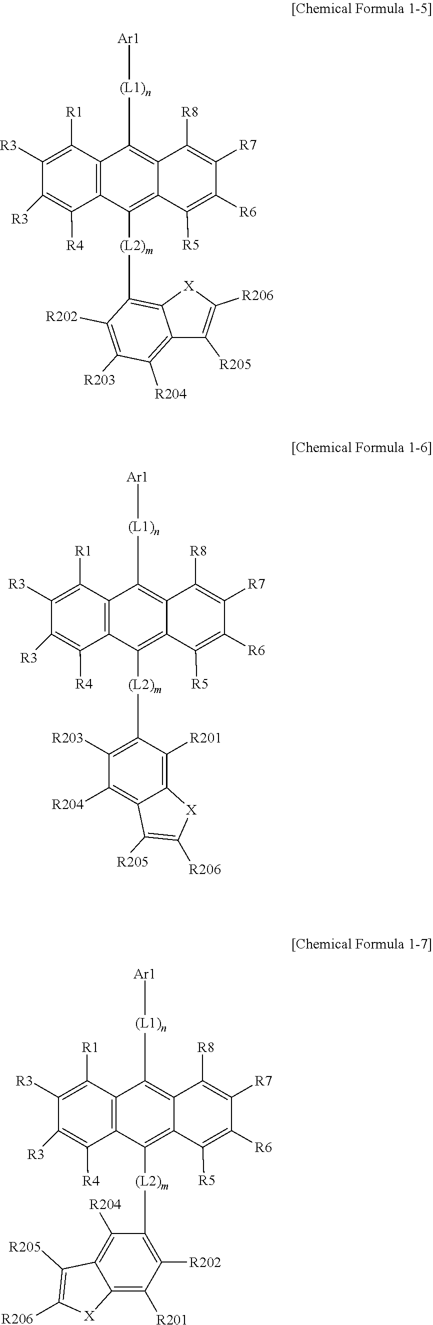

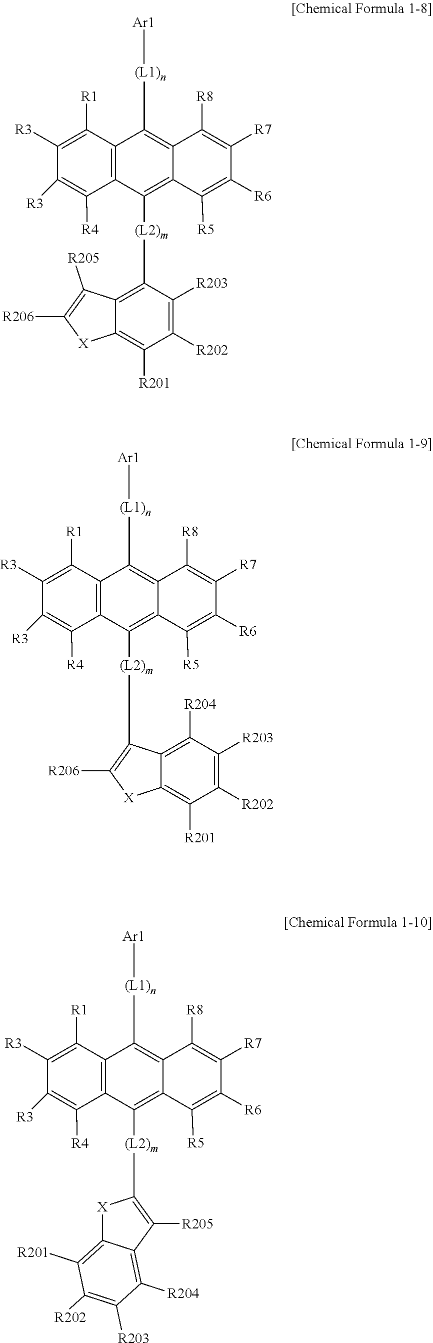

In Chemical Formulae 1-5 to 1-10,
the definitions of L1, L2, Ar1, X, n, and m are the same as the definitions in Chemical Formula 1 above,
R1 to R8 are each independently hydrogen; deuterium; a substituted or unsubstituted alkyl group; a substituted or unsubstituted cycloalkyl group; a substituted or unsubstituted aryl group; or a substituted or unsubstituted heteroaryl group, and
R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups.

In the present specification, Chemical Formula 1 above is Chemical Formula 1-5 above.

In the present specification, Chemical Formula 1 above is Chemical Formula 1-6 above.

In the present specification, Chemical Formula 1 above is Chemical Formula 1-7 above.

In the present specification, Chemical Formula 1 above is Chemical Formula 1-8 above.

In the present specification, Chemical Formula 1 above is Chemical Formula 1-9 above.

In the present specification, Chemical Formula 1 above is Chemical Formula 1-10 above.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group, a substituted or unsubstituted cycloalkyl group; a substituted or unsubstituted aryl group, or a substituted or unsubstituted heteroaryl group.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 30 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 30 carbon atoms, a substituted or unsubstituted aryl group having 6 to 30 carbon atoms, or a substituted or unsubstituted heteroaryl group having 2 to 30 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 20 carbon atoms, a substituted or unsubstituted aryl group having 6 to 20 carbon atoms, or a substituted or unsubstituted heteroaryl group having 2 to 20 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 10 carbon atoms, a substituted or unsubstituted aryl group having 6 to 10 carbon atoms, or a substituted or unsubstituted heteroaryl group having 2 to 10 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted phenyl group; or a naphthyl group unsubstituted or substituted with deuterium.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen or deuterium.

In an exemplary embodiment of the present specification, R1 to R10, which are not bonded to L1 in Chemical Formula 1-A above and are not bonded to L2 in Chemical Formula 1-B, are hydrogen.

In an exemplary embodiment of the present specification, R1 to R10, which are not bonded to L1 in Chemical Formula 1-A above and are not bonded to L2 in Chemical Formula 1-B, are deuterium.

In an exemplary embodiment of the present specification, L1 and L2 are the same as or different from each other, and are each independently a direct bond; a substituted or unsubstituted arylene group; or a substituted or unsubstituted heteroarylene group.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms; or a heteroarylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms; or a heteroarylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms; or a heteroarylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms.

In an exemplary embodiment of the present specification, L1 and L2 are the same as or different from each other, and are each independently a direct bond; an arylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms; or a heteroarylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms; or a biphenylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms; or a biphenylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms; or a biphenylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms.

In an exemplary embodiment of the present specification, L1 and L2 are the same as or different from each other, and are each independently a direct bond; a naphthylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms; or a biphenylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms.

In an exemplary embodiment of the present specification, L1 and L2 are the same as or different from each other, and are each independently a direct bond; a substituted or unsubstituted phenylene group; a substituted or unsubstituted biphenylene group; a substituted or unsubstituted terphenylene group; a substituted or unsubstituted naphthylene group; or a substituted or unsubstituted fluorenylene group.

In an exemplary embodiment of the present specification, L1 and L2 are the same as or different from each other, and are each independently a direct bond; a substituted or unsubstituted phenylene group; a substituted or unsubstituted naphthylene group; a substituted or unsubstituted biphenylene group; or a substituted or unsubstituted terphenylene group.

In an exemplary embodiment of the present specification, R101 to R110 are the same as or different from each other, and are each independently hydrogen; deuterium; a substituted or unsubstituted alkyl group having 1 to 30 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 30 carbon atoms; a substituted or unsubstituted aryl group having 6 to 30 carbon atoms; or a substituted or unsubstituted heteroaryl group having 2 to 30 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 20 carbon atoms; or a substituted or unsubstituted heteroaryl group having 2 to 20 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 10 carbon atoms; or a substituted or unsubstituted heteroaryl group having 2 to 10 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted iso-propyl group; a substituted or unsubstituted tert-butyl group; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted tetrahydronaphthalene group; a substituted or unsubstituted spirobifluorenyl group; a substituted or unsubstituted triphenylenyl group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group, and one of R101 to R108 is necessarily bonded to L1.

X is O or S.

X is O.

X is S.

In an exemplary embodiment of the present specification, R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R204 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 30 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 30 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and at least one pair adjacent to each other in R201 to R204 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 30 carbon atoms; a substituted or unsubstituted aryl group having 6 to 30 carbon atoms; or a substituted or unsubstituted heteroaryl group having 2 to 30 carbon atoms, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 30 carbon atoms; a substituted or unsubstituted aryl group having 6 to 30 carbon atoms; or a substituted or unsubstituted heteroaryl group having 2 to 30 carbon atoms, and at least one pair adjacent to each other in R201 to R204 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 20 carbon atoms; or a substituted or unsubstituted heteroaryl group having 2 to 20 carbon atoms, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 20 carbon atoms; or a substituted or unsubstituted heteroaryl group having 2 to 20 carbon atoms, and at least one pair adjacent to each other in R201 to R204 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 10 carbon atoms; a substituted or unsubstituted aryl group having 6 to 10 carbon atoms; or a substituted or unsubstituted heteroaryl group having 2 to 10 carbon atoms, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted alkyl group having 1 to 10 carbon atoms; a substituted or unsubstituted cycloalkyl group having 3 to 10 carbon atoms; a substituted or unsubstituted aryl group having 6 to 10 carbon atoms; or a substituted or unsubstituted heteroaryl group having 2 to 10 carbon atoms, and at least one pair adjacent to each other in R201 to R204 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted iso-isopropyl group; a substituted or unsubstituted tert-butyl group; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted tetrahydronaphthalene group; a substituted or unsubstituted adamantane group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group, and at least one pair adjacent to each other in R201 to R206 is bonded to each other to necessarily form one or more substituted or unsubstituted ring groups.

In an exemplary embodiment of the present specification, R201 to R206 are the same as or different from each other, and are each independently hydrogen; deuterium; a substituted or unsubstituted iso-isopropyl group; a substituted or unsubstituted tert-butyl group; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted tetrahydronaphthalene group; a substituted or unsubstituted adamantane group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group, and at least one pair adjacent to each other in R201 to R204 is bonded to each other to necessarily form one or more substituted or unsubstituted ring groups.

In an exemplary embodiment of the present specification, the substituted or unsubstituted ring group; or any one of R201 to R206 not forming the substituted or unsubstituted ring group is bonded to L2.

In an exemplary embodiment of the present specification, the substituted or unsubstituted ring group is bonded to L2.

In an exemplary embodiment of the present specification, any one of R201 to R206 not forming the substituted or unsubstituted ring group is bonded to L2.

In an exemplary embodiment of the present specification, n is an integer from 1 to 4.

In an exemplary embodiment of the present specification, n is 4.

In an exemplary embodiment of the present specification, n is 3.

In an exemplary embodiment of the present specification, n is 2.

In an exemplary embodiment of the present specification, n is 1.

In an exemplary embodiment of the present specification, m is an integer from 1 to 4.

In an exemplary embodiment of the present specification, m is 4.

In an exemplary embodiment of the present specification, m is 3.

In an exemplary embodiment of the present specification, m is 2.

In an exemplary embodiment of the present specification, m is 1.

According to an exemplary embodiment of the present specification, R9 of Chemical Formula 1 above is bonded to L1 of Chemical Formula 1-A above, and R10 of Chemical Formula 1 above is bonded to L2 of Chemical Formula 1-B above, and
all of R1 to R8 of Chemical Formula 1 above are deuterium.

According to an exemplary embodiment of the present specification, the deuterium substitution rate of Chemical Formula 1 is 15% or more.

According to an exemplary embodiment of the present specification, the deuterium substitution rate of Chemical Formula 1 is 30% or more.

According to an exemplary embodiment of the present specification, the deuterium substitution rate of Chemical Formula 1 is 40% or more.

According to an exemplary embodiment of the present specification, the deuterium substitution rate of Chemical Formula 1 is 50% or more.

According to an exemplary embodiment of the present specification, the deuterium substitution rate of Chemical Formula 1 is 60% or more.

According to an exemplary embodiment of the present specification, the deuterium substitution rate of Chemical Formula 1 is 70% or more.

According to an exemplary embodiment of the present specification, the deuterium substitution rate of Chemical Formula 1 is 80% or more.

According to an exemplary embodiment of the present specification, the deuterium substitution rate of Chemical Formula 1 is 90% or more.

According to an exemplary embodiment of the present specification, the deuterium substitution rate of Chemical Formula 1 is 100%.

According to an exemplary embodiment of the present specification, when Chemical Formula 1 includes deuterium, there are the following effects. Specifically, the physicochemical properties related to deuterium, such as the chemical bond length, are different from those of hydrogen, the van der Waals radius of deuterium is smaller than that of hydrogen because the stretching amplitude of the C-D bond is smaller than that of the C-H bond, and in general, it can be shown that the C-D bond is shorter and stronger than the C-H bond. Therefore, when hydrogen at a substitutable position in Chemical Formula 1 is substituted with deuterium, the energy of the ground state is reduced and the bond length between deuterium and carbon is shortened, resulting in a reduction in molecular hardcore volume, and accordingly, the electrical polarizability may be reduced, and the thin film volume may be increased by weakening the intermolecular interaction. In addition, these characteristics may have an effect of reducing the crystallinity of the thin film, that is, create an amorphous state, and may be generally effective for increasing the service life and driving characteristics of an organic light emitting device, and the heat resistance may be improved compared to organic light emitting devices in the related art.

In the present specification, “including deuterium, “deuteration” or “deuterated” means that hydrogen at a substitutable position of a compound is substituted with deuterium.

In the present specification, “overdeuterated” means a compound or group in which all hydrogens in the molecule are substituted with deuterium, and has the same meaning as “100% deuterated”.

In the present specification, “X % deuterated”, “X % degree of deuteration”, or “X % deuterium substitution rate” means that X % of the hydrogens at substitutable positions in the corresponding structure are substituted with deuterium. For example, when the corresponding structure is dibenzofuran, the dibenzofuran being “25% deuterated”, “25% degree of deuteration” of the dibenzofuran, or “25% deuterium substitution rate” of the dibenzofuran means that 2 of the 8 hydrogens at the substitutable positions of the dibenzofuran are substituted with deuterium.

In the present specification, the “degree of deuteration” or “deuterium substitution rate” may be confirmed by a known method such as nuclear magnetic resonance spectroscopy (¹H NMR), thin-layer chromatography/mass spectrometry (TLS/MS), or gas chromatography/mass spectrometry (GC/MS).

Specifically, when the “degree of deuteration” or “deuterium substitution rate” is analyzed using nuclear magnetic resonance spectroscopy (¹H NMR), the degree of deuteration or deuterium substitution rate may be calculated from the integral amount of the total peak through the integration ratio on 1H NMR by adding dimethylformamide (DMF) as an internal standard.

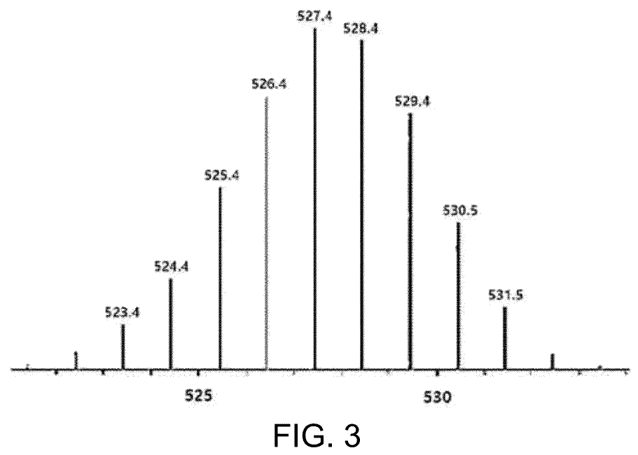

In addition, when the “degree of deuteration” or “deuterium substitution rate” is analyzed by thin-layer chromatography/mass spectrometry (TLS/MS), the substitution rate may be calculated based on the maximum value (median value) of the distribution of molecular weights at the end of the reaction. For example, when the degree of deuteration of the following Compound A is analyzed, the molecular weight of the following starting material is set to 506 and the maximum molecular weight (median value) of the following Compound A is set to 527 in the MS graph of FIG. 3, then it may be calculated that about 81% of the hydrogen has been deuterated because 21 hydrogens of the hydrogens (26 ea) at the substitutable positions in the following starting material have been substituted with deuterium.

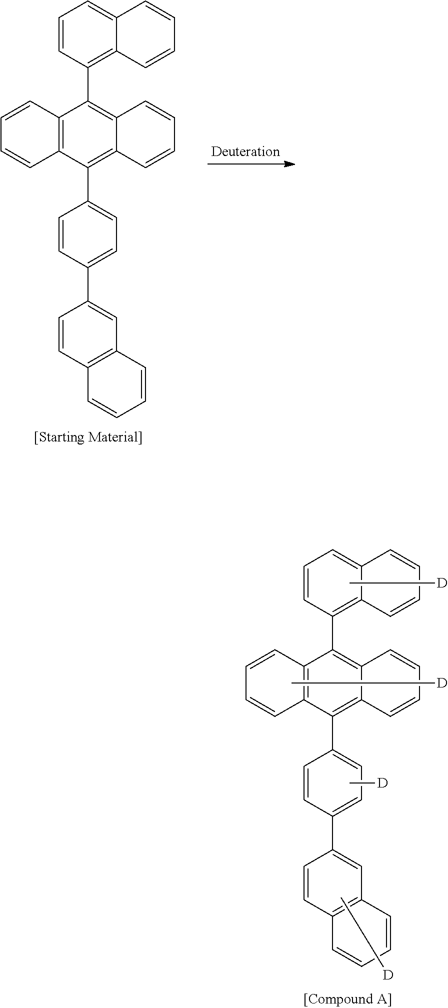

In the present specification, D means deuterium.

In an exemplary embodiment of the present specification, Chemical Formula 1 above is an organic compound of any one of the following compounds.

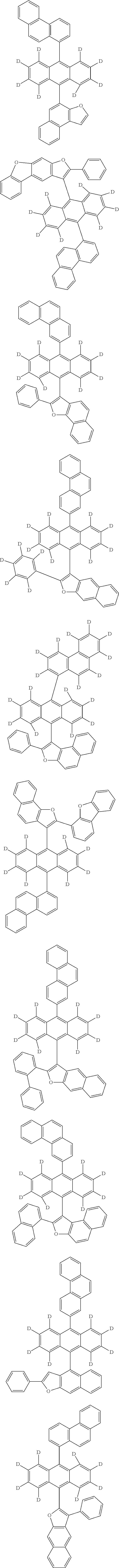

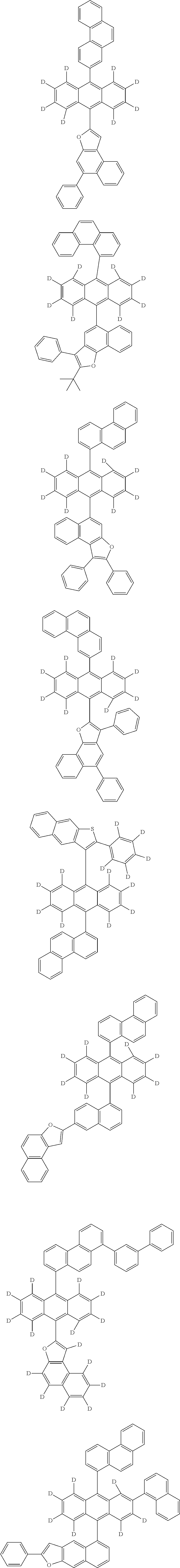

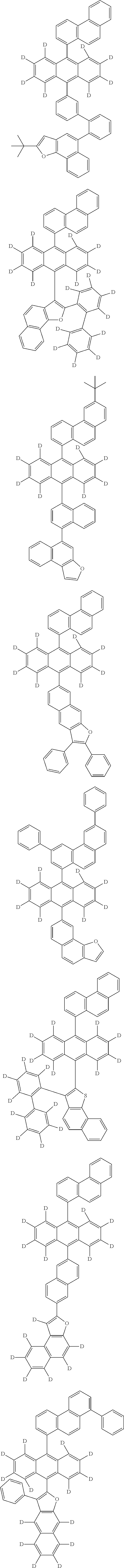

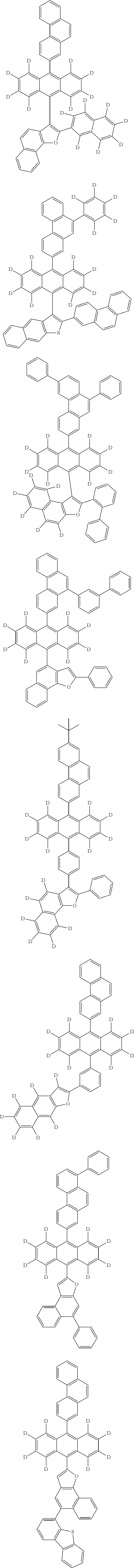

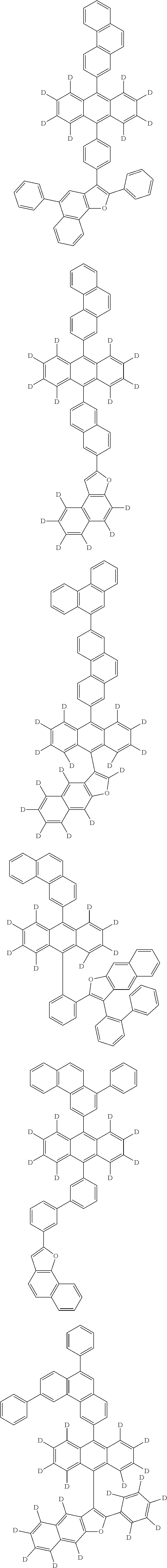

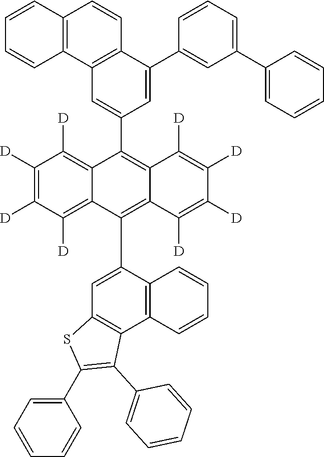 [SMILES: [2H]c1c([2H])c([2H])c2c(-c3cc4sc(-c5ccccc5)c(-c5ccccc5)c4c4ccccc34)c3c([2H])c([2H])c([2H])c([2H])c3c(-c3cc(-c4cccc(-c5ccccc5)c4)c4ccc5ccccc5c4c3)c2c1[2H]]

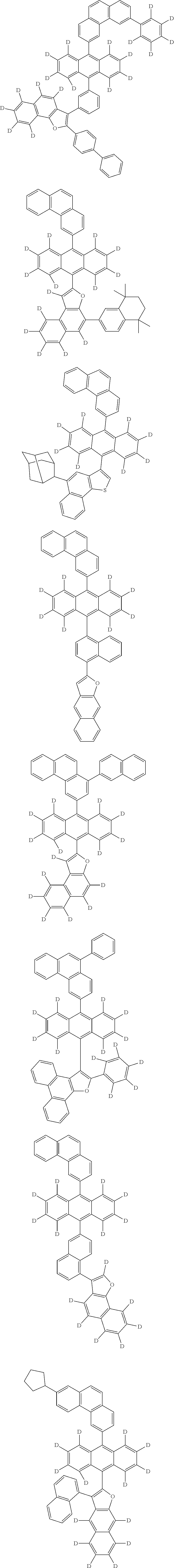

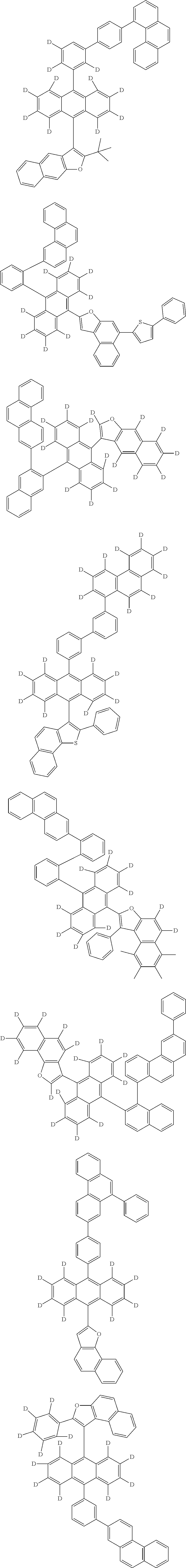

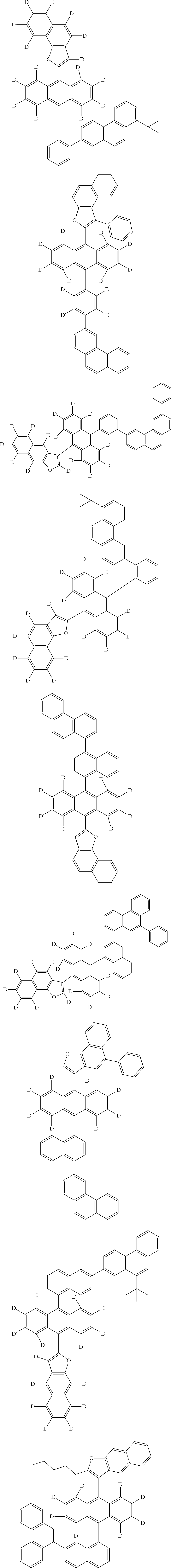

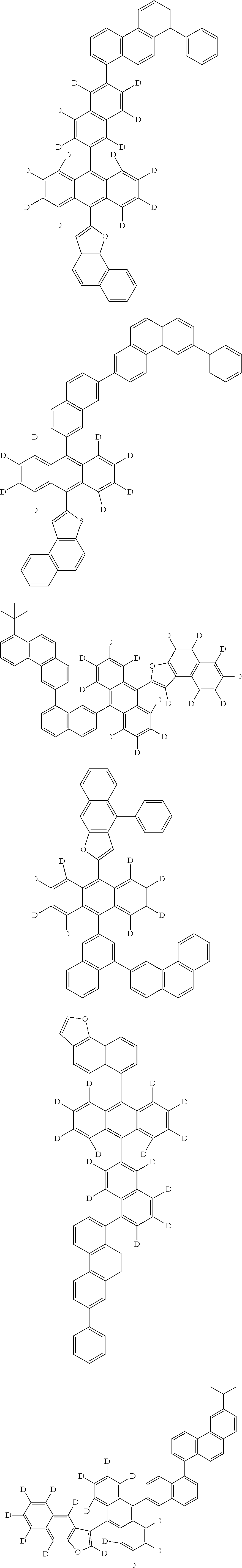

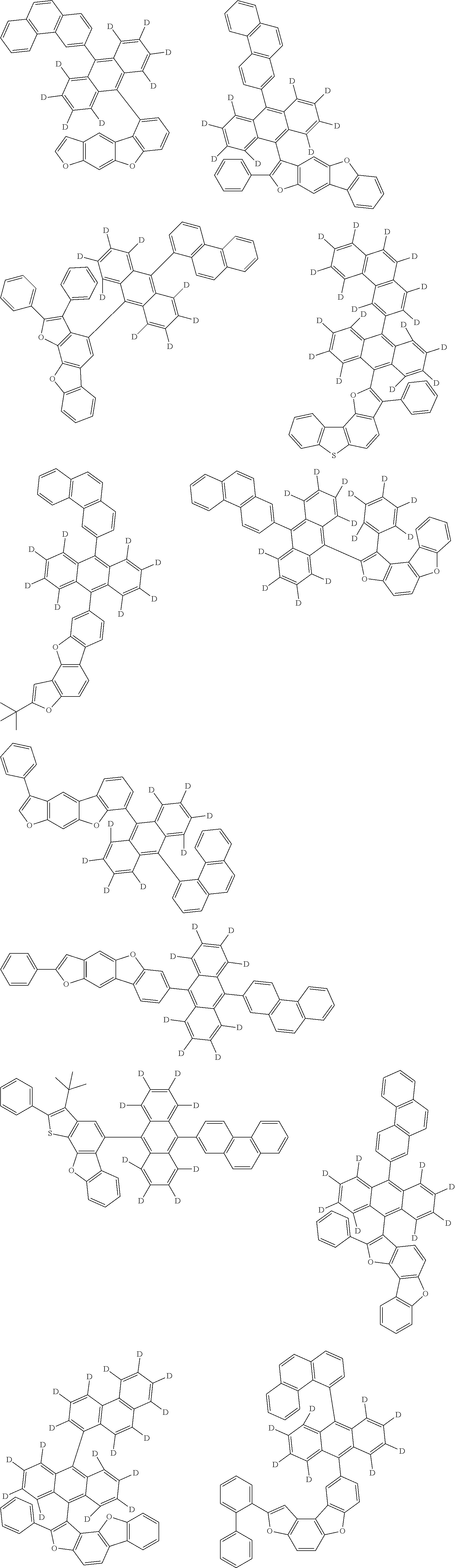

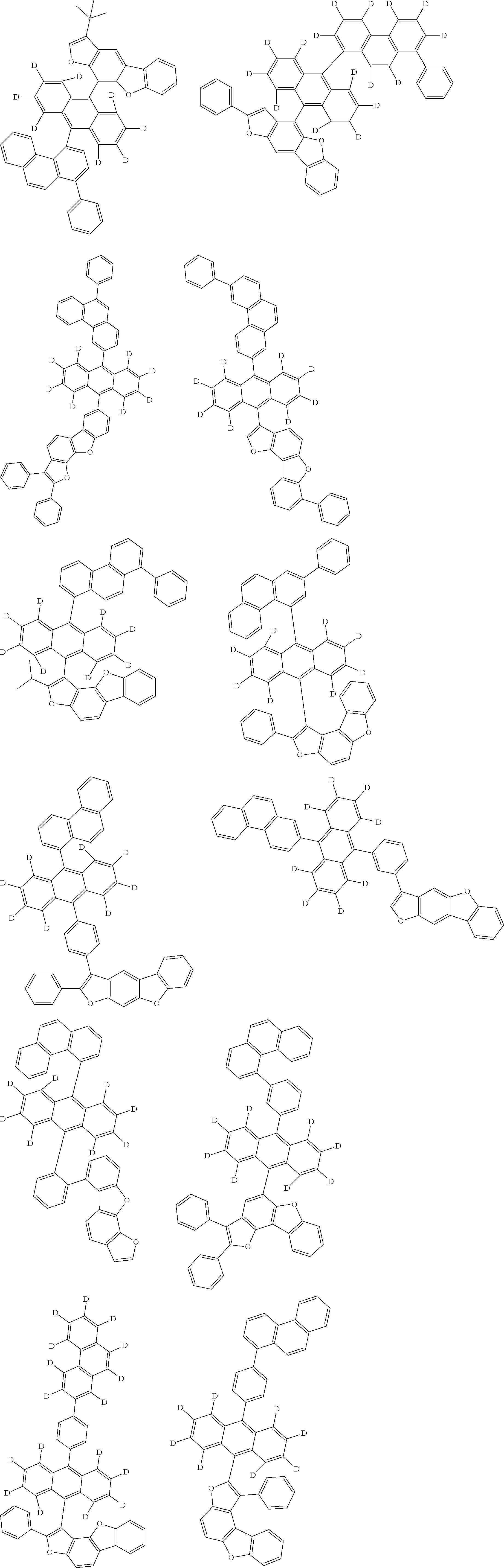

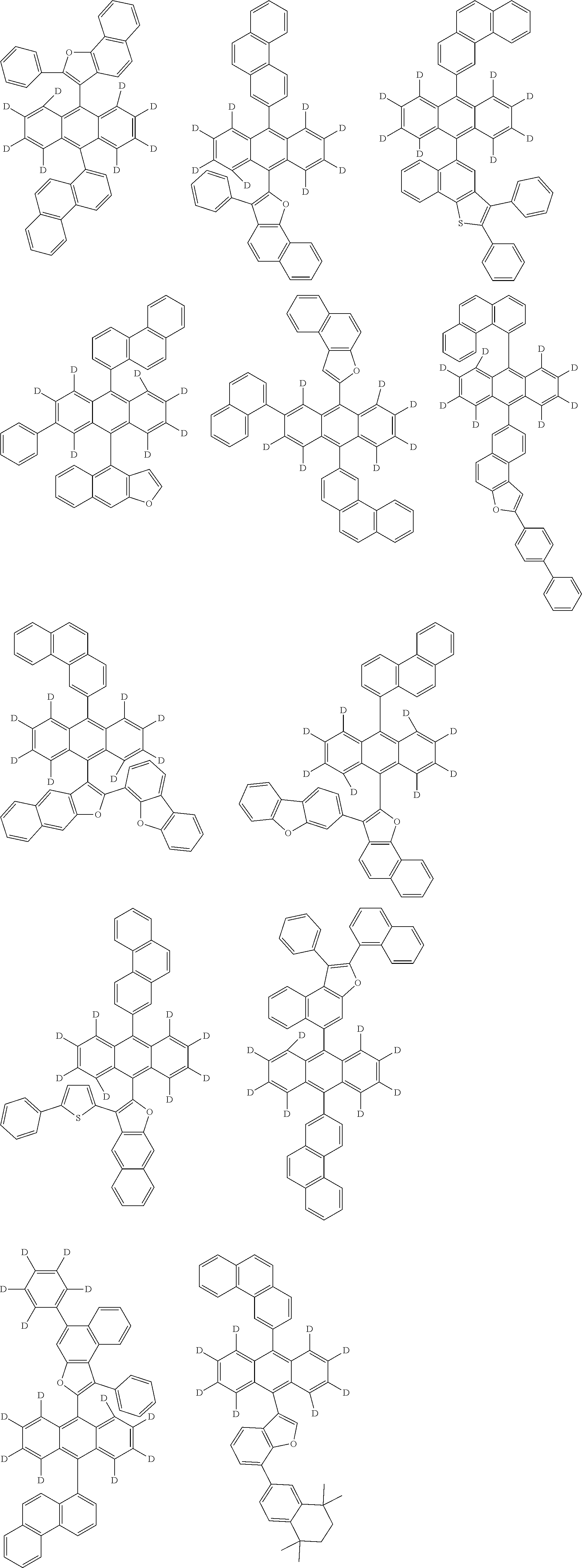

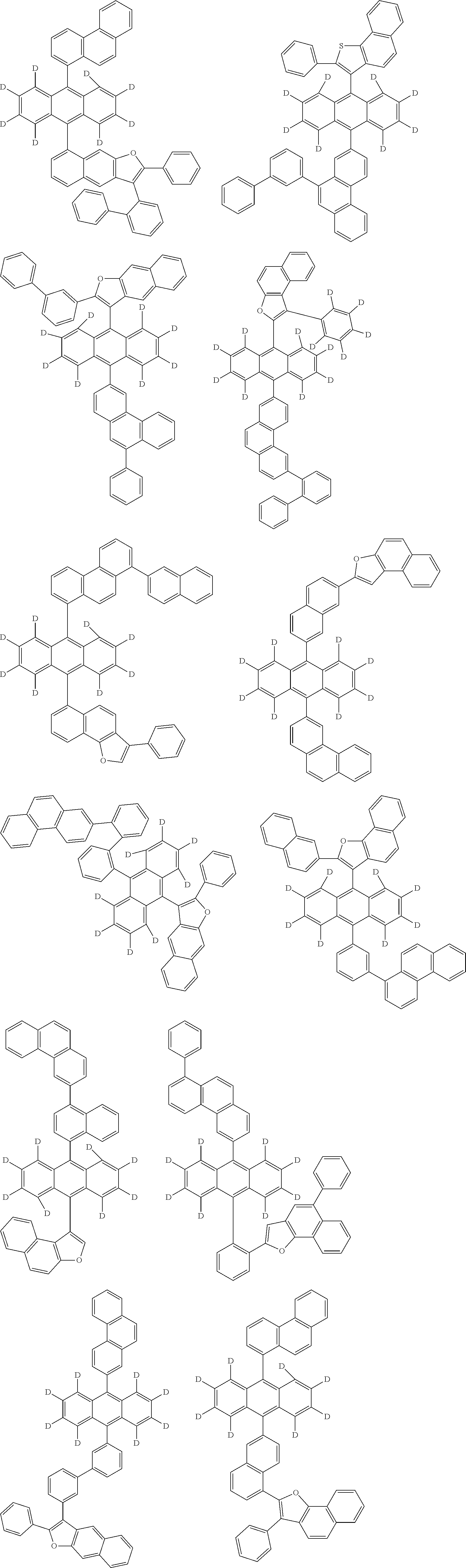

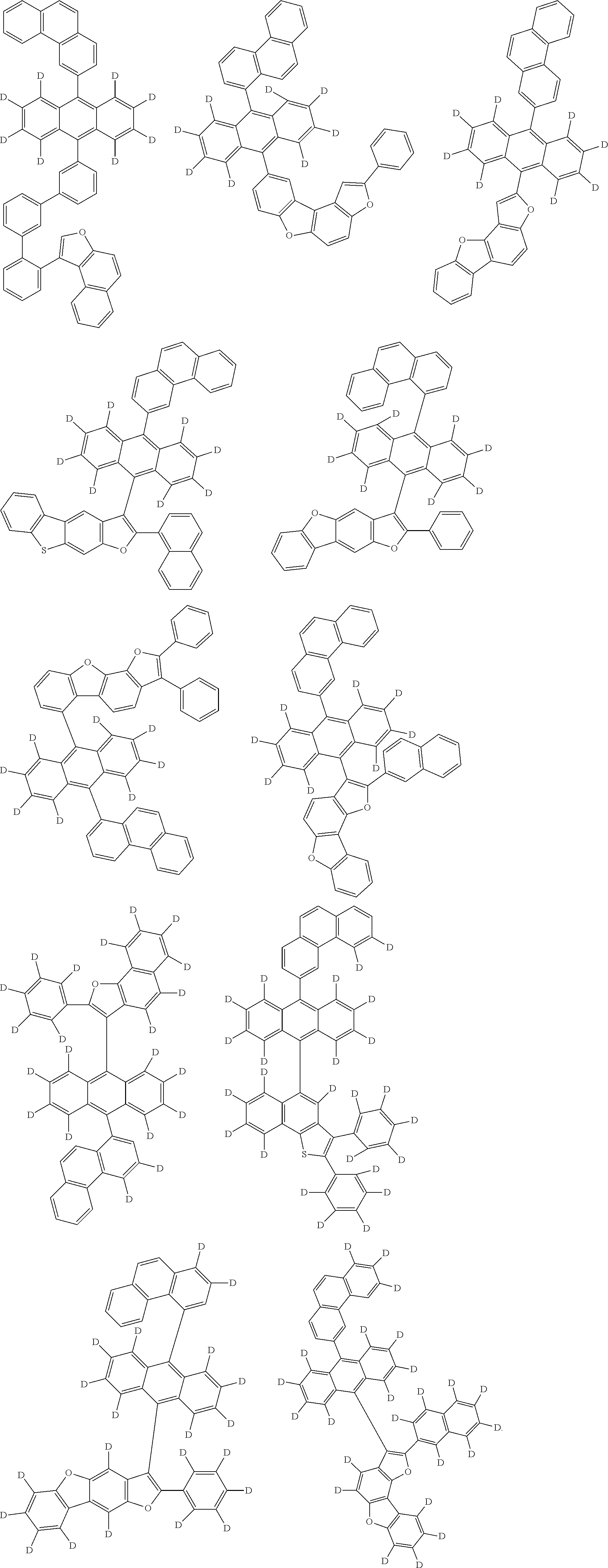

In the organic compound of Chemical Formula 1 according to an exemplary embodiment of the present specification, a core structure may be prepared as in the Preparation Examples described below. The substituent may be bonded by a method known in the art, and the kind and position of the substituent or the number of substituents may be changed according to the technology known in the art.

In the present specification, compounds having various energy bandgaps may be synthesized by introducing various substituents into the core structure of the organic compound of Chemical Formula 1. Further, in the present specification, various substituents may be introduced into the core structures having the structure described above to adjust the HOMO and LUMO energy levels of a compound.

In addition, the present specification provides an organic light emitting device including the above-described organic compound.

When one member is disposed “on” another member in the present specification, this includes not only a case where the one member is brought into contact with another member, but also a case where still another member is present between the two members.

The organic light emitting device according to the present specification is an organic light emitting device including: an anode; a cathode; and an organic material layer having one or more layers provided between the anode and the cathode, in which one or more layers of the organic material layer include the above-described organic compound represented by Chemical Formula 1.

The organic material layer of the organic light emitting device of the present specification may be composed of a single-layered structure, but may also be composed of a multi-layered structure in which two or more organic material layers are stacked. For example, the organic light emitting device of the present invention may have a structure including one or more layers of a hole transport layer, a hole injection layer, a hole adjusting layer, a hole transport and injection layer, an electron transport layer, an electron injection layer, an electron adjusting layer, and an electron transport and injection layer as organic material layers. However, the structure of the organic light emitting device of the present specification is not limited thereto, and may include a fewer or greater number of organic material layers.

In the organic light emitting device of the present specification, the organic light emitting device includes a light emitting layer, and the light emitting layer may include the organic compound.

For example, the organic compound of Chemical Formula 1 may be included as a host of the light emitting layer.

According to an exemplary embodiment of the present specification, the light emitting layer includes a dopant, and the dopant includes a fluorescent dopant.

According to an exemplary embodiment of the present specification, the fluorescent dopant is a pyrene-based compound or a bipyrene-based compound.

According to an exemplary embodiment of the present specification, the bipyrene-based compound includes a boron-based compound.

According to an exemplary embodiment of the present specification, the light emitting layer further includes one or more hosts different from the compound of Chemical Formula 1.

In an exemplary embodiment of the present specification, the host different from the compound of Chemical Formula 1 includes a compound of the following Chemical Formula H-1.

In an exemplary embodiment of the present specification, the light emitting layer of the organic light emitting device includes the organic compound as a host and further includes one host represented by the following Chemical Formula H-1 different from the organic compound.

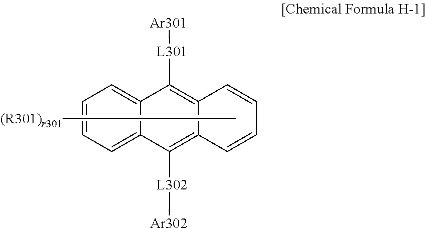

In Chemical Formula H-1,
L301 and L302 are the same as or different from each other, and are each independently a direct bond; a substituted or unsubstituted arylene group; or a substituted or unsubstituted heterocyclic group,
Ar301 and Ar302 are the same as or different from each other, and are each independently hydrogen; deuterium; a substituted or unsubstituted aryl group; or a substituted or unsubstituted heterocyclic group,
R301 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r301 is an integer from 1 to 7, and when r301 is 2 or higher, two or more R301's are the same as or different from each other.

According to an exemplary embodiment of the present specification, Chemical Formula H-1 is represented by the following compound.

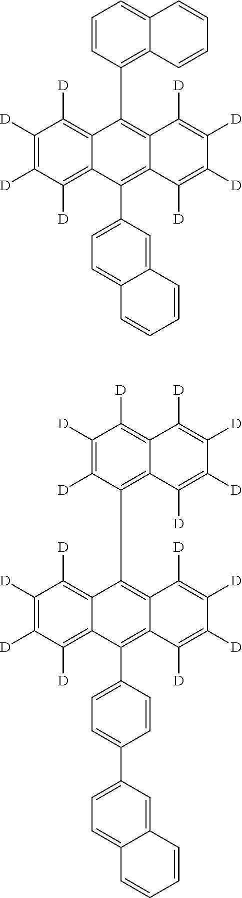 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3c4c([2H])c([2H])c([2H])c([2H])c4c(-c4ccc(-c5ccc6ccccc6c5)cc4)c4c([2H])c([2H])c([2H])c([2H])c34)c([2H])c([2H])c([2H])c2c1[2H]]

Any anthracene-based host used in the art can be used without limitation as long as the host different from the compound of Chemical Formula 1 is different from Chemical Formula 1, and the host is not limited thereto.

According to an exemplary embodiment of the present specification, the light emitting layer includes a host and a dopant.

According to an exemplary embodiment of the present specification, the light emitting layer includes a host and a dopant, and the host includes the compound represented by Chemical Formula 1.

According to an exemplary embodiment of the present specification, the dopant is a blue dopant.

According to an exemplary embodiment of the present specification, the organic light emitting device is a blue organic light emitting device.

According to an exemplary embodiment of the present specification, the light emitting layer includes a host and a dopant, the host includes the compound represented by Chemical Formula 1, and the dopant includes one or more selected from a pyrene-based compound and a bipyrene-based compound.

According to an exemplary embodiment of the present specification, the light emitting layer includes a host and a dopant, and for the light emitting layer, the host and the dopant have a weight ratio of 8:2 to 99:1.

According to an exemplary embodiment of the present specification, the light emitting layer includes a host and a dopant, and for the light emitting layer, the host and the dopant have a weight ratio of 8:2 to 95:5.

According to an exemplary embodiment of the present specification, the light emitting layer includes a host and a dopant, and for the light emitting layer, the host and the dopant have a weight ratio of 90:10 to 99:1.

According to another exemplary embodiment of the present specification, the light emitting layer further includes a capping layer provided on a surface of at least one of the first electrode and the second electrode opposite to the surface facing the organic material layer.

The capping layer is formed to prevent a considerable amount of light from being lost through total reflection of light in the organic light emitting device, and the capping layer has the performance capable of sufficiently protecting the underlying negative electrode and light emitting layer from external moisture penetration or contamination, and has a high refractive index, and thus may prevent light loss caused by total reflection.

According to still another exemplary embodiment of the present specification, the capping layer may be provided on each of a surface of the first electrode opposite to the surface facing the organic layer and a surface of the second electrode opposite to the surface facing the organic layer.

According to yet another exemplary embodiment of the present specification, the capping layer may be provided on a surface of the first electrode opposite to the surface facing the organic layer.

According to still yet another exemplary embodiment of the present specification, the capping layer may be provided on a surface of the second electrode opposite to the surface facing the organic layer.

According to an exemplary embodiment of the present specification, the organic light emitting device further includes one or two or more layers selected from the group consisting of a hole injection layer, a hole transport layer, a light emitting layer, an electron transport layer, an electron injection layer, an electron adjusting layer, and a hole adjusting layer.

According to an exemplary embodiment of the present specification, the organic light emitting device includes: a first electrode; a second electrode; a light emitting layer provided between the first electrode and the second electrode; and an organic material layer having two or more layers provided between the light emitting layer and the first electrode, or between the light emitting layer and the second electrode.

According to an exemplary embodiment of the present specification, as the organic material layer having two or more layers between the light emitting layer and the first electrode, or between the light emitting layer and the second electrode, two or more may be selected from the group consisting of a light emitting layer, a hole transport layer, a hole injection layer, a hole injection and transport layer, a hole adjusting layer, an electron adjusting layer, an electron injection layer, an electron transport layer, and an electron injection and transport layer.

According to an exemplary embodiment of the present specification, a hole transport layer having two or more layers is included between the light emitting layer and the first electrode. The hole transport layer having two or more layers may include materials which are the same as or different from each other.

According to an exemplary embodiment of the present specification, the first electrode is an anode or a cathode.

According to an exemplary embodiment of the present specification, the second electrode is a cathode or an anode.

According to an exemplary embodiment of the present specification, the organic light emitting device may be a normal type organic light emitting device in which an anode, an organic material layer having one or more layers, and a cathode are sequentially stacked on a substrate.

According to an exemplary embodiment of the present specification, the organic light emitting device may be an inverted type organic light emitting device in which a cathode, an organic material layer having one or more layers, and an anode are sequentially stacked on a substrate.

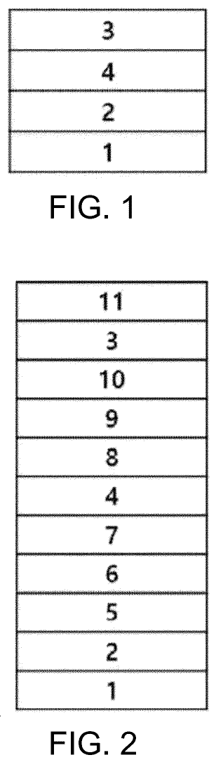

For example, the structure of the organic light emitting device according to an exemplary embodiment of the present specification is exemplified in FIGS. 1 and 2. FIGS. 1 and 2 exemplify an organic light emitting device, and the organic light emitting device is not limited thereto.

FIG. 1 exemplifies a structure of an organic light emitting device in which a first electrode 2, a light emitting layer 4, and a second electrode 3 are sequentially stacked on a substrate 1. The compound is included in the light emitting layer.

FIG. 2 exemplifies the structure of an organic light emitting device in which a first electrode 2, a hole injection layer 5, a hole transport layer 6, a hole adjusting layer 7, a light emitting layer 4, an electron adjusting layer 8, an electron transport layer 9, an electron injection layer 10, a second electrode 3, and a capping layer 11 are sequentially stacked on a substrate 1. The compound is included in the light emitting layer.

The organic light emitting device of the present specification may be manufactured by the materials and methods known in the art, except that the light emitting layer is included or the light emitting layer includes the compound, that is, the compound of Chemical Formula 1.

When the organic light emitting device includes a plurality of organic material layers, the organic material layers may be formed with materials the same as or different from each other.

For example, the organic light emitting device of the present specification may be manufactured by sequentially stacking a first electrode, an organic material layer, and a second electrode on a substrate. In this case, the organic light emitting device may be manufactured by depositing a metal or a metal oxide having conductivity, or an alloy thereof on a substrate to form an anode, forming an organic material layer including a hole injection layer, a hole transport layer, a light emitting layer, and an electron transport layer thereon, and then depositing a material, which may be used as a cathode, thereon, by using a physical vapor deposition (PVD) method such as sputtering or e-beam evaporation. In addition to the method described above, an organic light emitting device may be manufactured by sequentially depositing a second electrode material, an organic material layer, and a first electrode material on a substrate.

Further, the compound represented by Chemical Formula 1 may be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In addition to the method described above, an organic light emitting device may be made by sequentially depositing a second electrode material, an organic material layer, and a first electrode material on a substrate. However, the manufacturing method is not limited thereto.

As the first electrode material, materials having a high work function are usually preferred so as to facilitate the injection of holes into an organic material layer. Examples thereof include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene](PEDOT), polypyrrole, and polyaniline; and the like, but are not limited thereto.

As the second electrode material, materials having a low work function are usually preferred so as to facilitate the injection of electrons into an organic material layer. Examples thereof include: a metal, such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The light emitting layer may include a host material and a dopant material. When an additional light emitting layer is included in addition to a light emitting layer including the compound of Chemical Formula 1 according to an exemplary embodiment of the present specification, examples of a host material include a fused and/or non-fused aromatic ring derivative, a hetero ring-containing compound, or the like. Specific examples of the fused aromatic ring derivative include anthracene derivatives, pyrene derivatives, naphthalene derivatives, pentacene derivatives, phenanthrene compounds, fluoranthene compounds, and the like, and specific examples of the hetero ring-containing compound include dibenzofuran derivatives, ladder-type furan compounds, pyrimidine derivatives, and the like, but the examples are not limited thereto.

According to an exemplary embodiment of the present specification, the host includes the compound represented by Chemical Formula 2, but is not limited thereto.

Examples of the dopant material include an aromatic amine derivative, a styrylamine compound, a boron complex, a fluoranthene compound, a metal complex, and the like. Specifically, the aromatic amine derivative is a fused aromatic ring derivative having a substituted or unsubstituted arylamine group, and examples thereof include pyrene, anthracene, chrysene, periflanthene, and the like having an arylamine group. Further, the styrylamine compound is a compound in which a substituted or unsubstituted arylamine is substituted with at least one arylvinyl group, and one or two or more substituents selected from the group consisting of an aryl group, a silyl group, an alkyl group, a cycloalkyl group, and an arylamine group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

According to an exemplary embodiment of the present specification, the dopant material includes a compound of the following Chemical Formula D-1 or D-2, but is not limited thereto.

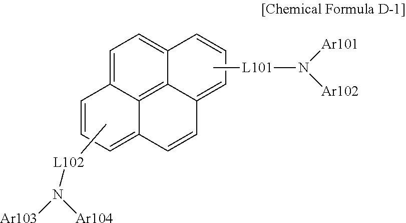

In Chemical Formula D-1,
L101 and L102 are the same as or different from each other, and are each independently a direct bond; or a substituted or unsubstituted arylene group, and
Ar101 to Ar104 are the same as or different from each other, and are each independently a substituted or unsubstituted aryl group; or a substituted or unsubstituted heteroaryl group,

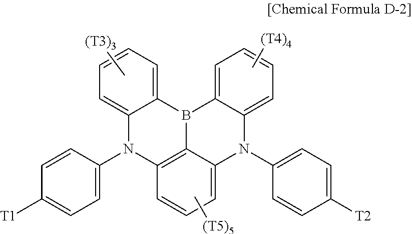

in Chemical Formula D-2,
T1 to T5 are the same as or different from each other, and are each independently hydrogen; a substituted or unsubstituted alkyl group; a substituted or unsubstituted amine group; or a substituted or unsubstituted aryl group,
t3 and t4 are each an integer from 1 to 4,
t5 is an integer from 1 to 3,
when t3 is 2 or higher, two or more T3's are the same as or different from each other,
when t4 is 2 or higher, two or more T4's are the same as or different from each other, and
when t5 is 2 or higher, two or more T5's are the same as or different from each other.

According to an exemplary embodiment of the present specification, L101 and L102 are a direct bond.

According to an exemplary embodiment of the present specification, Ar101 to Ar104 are the same as or different from each other, and are each independently a substituted or unsubstituted monocyclic or polycyclic aryl group having 6 to 30 carbon atoms; or a substituted or unsubstituted monocyclic or polycyclic heteroaryl group having 2 to 30 carbon atoms.

According to an exemplary embodiment of the present specification, Ar101 to Ar104 are the same as or different from each other, and are each independently a monocyclic or polycyclic aryl group having 6 to 30 carbon atoms, which is unsubstituted or substituted with a straight-chained or branched alkyl group having 1 to 30 carbon atoms; or a monocyclic or polycyclic heteroaryl group having 2 to 30 carbon atoms.

According to an exemplary embodiment of the present specification, Ar101 to Ar104 are the same as or different from each other, and are each independently a phenyl group substituted with a methyl group; or a dibenzofuran group.

According to an exemplary embodiment of the present specification, Chemical Formula D-1 is represented by the following compound.

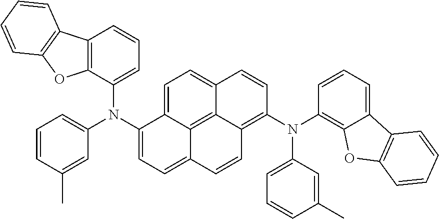 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1]

According to an exemplary embodiment of the present specification, T1 to T5 are the same as or different from each other, and are each independently hydrogen; a substituted or unsubstituted straight-chained or branched alkyl group having 1 to 30 carbon atoms; a substituted or unsubstituted monocyclic or polycyclic arylamine group having 6 to 30 carbon atoms; or a substituted or unsubstituted monocyclic or polycyclic aryl group having 6 to 30 carbon atoms.

According to an exemplary embodiment of the present specification, T1 to T5 are the same as or different from each other, and are each independently hydrogen; a straight-chained or branched alkyl group having 1 to 30 carbon atoms; a monocyclic or polycyclic arylamine group having 6 to 30 carbon atoms; or a monocyclic or polycyclic aryl group having 6 to 30 carbon atoms, which is unsubstituted or substituted with a straight-chained or branched alkyl group having 1 to 30 carbon atoms.

According to an exemplary embodiment of the present specification, T1 to T5 are the same as or different from each other, and are each independently hydrogen; a methyl group; a tert-butyl group; a diphenylamine group; or a phenyl group which is unsubstituted or substituted with a methyl group or tert-butyl group.

According to an exemplary embodiment of the present specification, Chemical Formula D-2 is represented by the following compound.

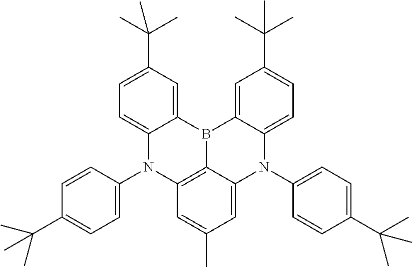 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

The hole injection layer is a layer which accepts holes from an electrode. It is preferred that the hole injection material has an ability to transport holes, and has an effect of accepting holes from an anode and an excellent hole injection effect for a light emitting layer or a light emitting material. Further, the hole injection material is preferably a material which is excellent in ability to prevent excitons produced from a light emitting layer from moving to an electron injection layer or an electron injection material. In addition, the hole injection material is preferably a material which is excellent in ability to form a thin film. In addition, the highest occupied molecular orbital (HOMO) of the hole injection material is preferably a value between the work function of the anode material and the HOMO of the neighboring organic material layer. Specific examples of the hole injection material include: metal porphyrin, oligothiophene, and arylamine-based organic materials; hexanitrile hexaazatriphenylene-based organic materials; quinacridone-based organic materials; perylene-based organic materials; polythiophene-based conductive polymers such as anthraquinone and polyaniline; and the like, but are not limited thereto.

According to an exemplary embodiment of the present specification, the hole injection layer includes a compound represented by the following Chemical Formula HI-1, but is not limited thereto.

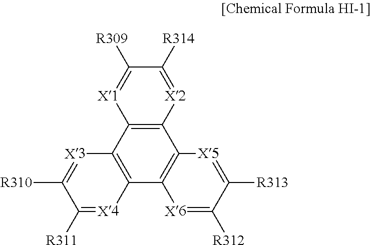

In Chemical Formula HI-1,
at least one of X′1 to X′6 is N, and the others are CH, and
R309 to R314 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a cyano group.

According to an exemplary embodiment of the present specification, Chemical Formula HI-1 is represented by the following compound.

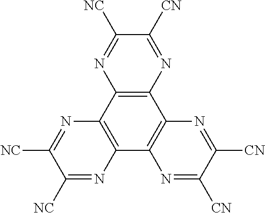 [SMILES: N#Cc1nc2c3nc(C#N)c(C#N)nc3c3nc(C#N)c(C#N)nc3c2nc1C#N]

The hole transport layer is a layer which accepts holes from a hole injection layer and transports the holes to a light emitting layer. A hole transport material is preferably a material having high hole mobility which may accept holes from an anode or a hole injection layer and transfer the holes to a light emitting layer. Specific examples thereof include arylamine-based organic materials, conductive polymers, block copolymers having both conjugated portions and non-conjugated portions, and the like, but are not limited thereto.

According to an exemplary embodiment of the present specification, the hole transport layer or hole adjusting layer includes a compound of following Chemical Formula HT-1, but is not limited thereto.

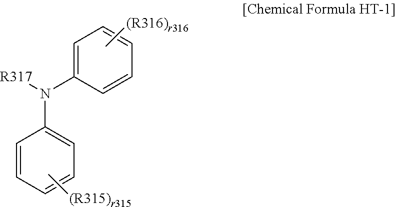

In Chemical Formula HT-1,
R315 to R317 are the same as or different from each other, and are each independently any one selected from the group consisting of hydrogen; deuterium; a substituted or unsubstituted alkyl group; a substituted or unsubstituted aryl group; a substituted or unsubstituted heteroaryl group; and a combination thereof, or are bonded to an adjacent group to form a substituted or unsubstituted ring,
r315 is an integer from 1 to 5, and when r315 is 2 or higher, two or more R315's are the same as or different from each other, and
r316 is an integer from 1 to 5, and when r316 is 2 or higher, two or more R316's are the same as or different from each other.

According to an exemplary embodiment of the present specification, R317 is any one selected from the group consisting of a substituted or unsubstituted aryl group; a substituted or unsubstituted heteroaryl group; and a combination thereof.

According to an exemplary embodiment of the present specification, R317 is any one selected from the group consisting of a carbazole group; a phenyl group; a biphenyl group; a fluorene group; and a combination thereof.

According to an exemplary embodiment of the present specification, R315 and R316 are the same as or different from each other, and are each independently a substituted or unsubstituted aryl group, or are bonded to an adjacent group to form an aromatic hydrocarbon ring substituted with an alkyl group.

According to an exemplary embodiment of the present specification, R315 and R316 are the same as or different from each other, and are each independently a phenyl group or a phenanthrene group, or are bonded to an adjacent group to form indene substituted with a methyl group.

According to an exemplary embodiment of the present specification, Chemical Formula HT-1 is represented by any one of the following compounds.

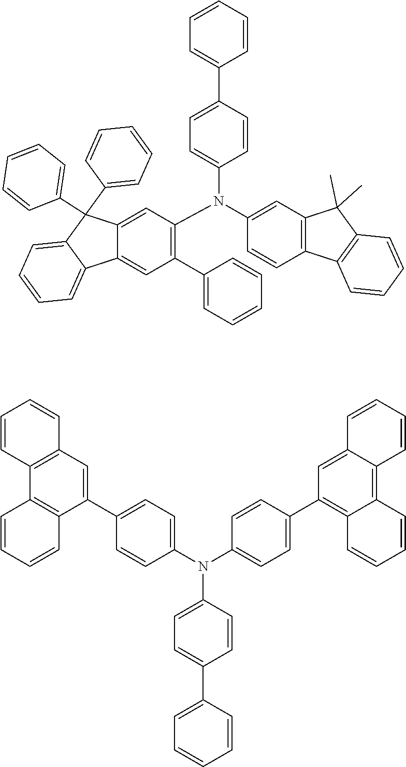 [SMILES: CC1(C)c2ccccc2-c2ccc(N(c3ccc(-c4ccccc4)cc3)c3cc4c(cc3-c3ccccc3)-c3ccccc3C4(c3ccccc3)c3ccccc3)cc21 ; c1ccc(-c2ccc(N(c3ccc(-c4cc5ccccc5c5ccccc45)cc3)c3ccc(-c4cc5ccccc5c5ccccc45)cc3)cc2)cc1]

The hole adjusting layer is a layer which may improve the service life and efficiency of a device by adjusting holes transported from a hole transport layer such that the holes are smoothly injected into a light emitting layer, and preventing electrons injected from an electron injection layer from passing through a light emitting layer and entering a hole injection layer. As the hole adjusting layer, the publicly-known material can be used without limitation, and the hole adjusting layer may be formed between a light emitting layer and a hole injection layer, between a light emitting layer and a hole transport layer, or between a light emitting layer and a layer which simultaneously injects and transports holes.

As a material for the hole adjusting layer, the examples of Chemical Formula HT-I may be applied, but the material is not limited thereto.

The electron adjusting layer is a layer that adjusts electrons transferred from the electron transport layer to be smoothly injected into the light emitting layer, and known materials can be used without limitation.

According to an exemplary embodiment of the present specification, the electron adjusting layer includes a compound of the following Chemical Formula EG-1, but is not limited thereto.

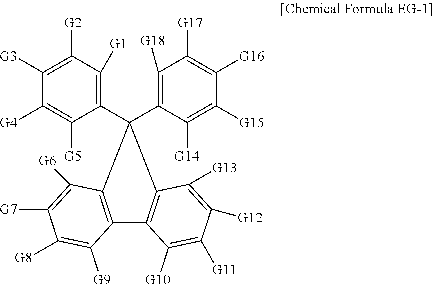

In Chemical Formula EG-1,
at least one of G1 to G18 is -L5-Ar5, the others are hydrogen, or G1 and G18 are linked through -L51- to form a substituted or unsubstituted ring,
L5 is a direct bond; or a substituted or unsubstituted arylene group,
Ar5 is a substituted or unsubstituted aryl group; or a substituted or unsubstituted heteroaryl group, and
L51 is O; or S.

According to an exemplary embodiment of the present specification, L51 is O.

According to an exemplary embodiment of the present specification, L51 is S.

According to an exemplary embodiment of the present specification, G1 and G18 are linked through -L51- to form a substituted or unsubstituted hetero ring.

According to an exemplary embodiment of the present specification, G1 and G18 are linked through -L51- to form a substituted or unsubstituted xanthene ring; or a substituted or unsubstituted thioxanthene ring.

According to an exemplary embodiment of the present specification, G1 and G18 are linked through —O— to form a substituted or unsubstituted xanthene ring.

According to an exemplary embodiment of the present specification, G1 and G18 are linked through —S— to form a substituted or unsubstituted thioxanthene ring.

According to an exemplary embodiment of the present specification, G1 and G18 are linked through —O— to form a xanthene ring.

According to an exemplary embodiment of the present specification, G1 and G18 are linked through —S— to form a thioxanthene ring.

According to an exemplary embodiment of the present specification, L5 is a direct bond; or a substituted or unsubstituted monocyclic or polycyclic arylene group having 6 to 30 carbon atoms.

According to an exemplary embodiment of the present specification, L5 is a direct bond; or a substituted or unsubstituted monocyclic or polycyclic arylene group having 6 to 20 carbon atoms.

According to an exemplary embodiment of the present specification, L5 is a direct bond; or a monocyclic or polycyclic arylene group having 6 to 30 carbon atoms.

According to an exemplary embodiment of the present specification, L5 is a direct bond; or a monocyclic or polycyclic arylene group having 6 to 20 carbon atoms.

According to an exemplary embodiment of the present specification, L5 is a direct bond; or a phenylene group.

According to an exemplary embodiment of the present specification, Ar5 is a substituted or unsubstituted triazine group.

According to an exemplary embodiment of the present specification, Ar5 is a triazine group which is unsubstituted or substituted with a monocyclic or polycyclic aryl group having 6 to 30 carbon atoms.

According to an exemplary embodiment of the present specification, Ar5 is a triazine group substituted with a phenyl group.

According to an exemplary embodiment of the present specification, EG-1 is represented by the following compound.

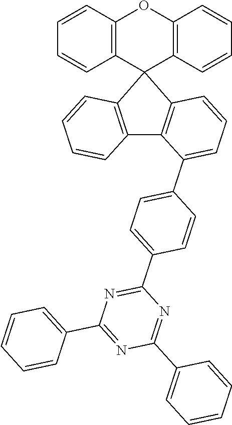 [SMILES: c1ccc(-c2nc(-c3ccccc3)nc(-c3ccc(-c4cccc5c4-c4ccccc4C54c5ccccc5Oc5ccccc54)cc3)n2)cc1]

The electron transport layer is a layer which accepts electrons from an electron injection layer and transports the electrons to a light emitting layer. An electron transport material is preferably a material having high electron mobility which may proficiently accept electrons from a cathode and transfer the electrons to a light emitting layer. Specific examples thereof include: Al complexes of 8-hydroxyquinoline; complexes including Alq3; organic radical compounds; hydroxyflavone-metal complexes; and the like, but are not limited thereto. The electron transport layer may be used with any desired cathode material, as used according to the related art. In particular, an appropriate cathode material is a typical material which has a low work function, followed by an aluminum layer or a silver layer. Specific examples thereof include cesium, barium, calcium, ytterbium, and samarium, in each case followed by an aluminum layer or a silver layer.

According to an exemplary embodiment of the present specification, the electron transport layer includes a compound of the following Chemical Formula ET-1, but is not limited thereto.

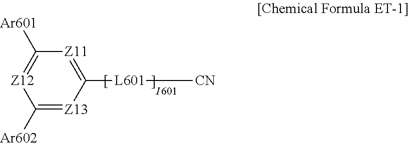

In Chemical Formula ET-1,
at least one of Z11 to Z13 is N, and the others are CH,
L601 is a direct bond; a substituted or unsubstituted arylene group; or a substituted or unsubstituted heteroarylene group,
Ar601 and Ar602 are the same as or different from each other, and are each independently a substituted or unsubstituted aryl group; or a substituted or unsubstituted heteroaryl group, and
1601 is an integer from 1 to 5, and when 1601 is 2 or higher, two or more L601's are the same as or different from each other.

According to an exemplary embodiment of the present specification, L601 is a substituted or unsubstituted monocyclic or polycyclic arylene group having 6 to 30 carbon atoms.

According to an exemplary embodiment of the present specification, L601 is a phenylene group; a biphenylylene group; or a naphthylene group.

According to an exemplary embodiment of the present specification, Ar601 and Ar602 are the same as or different from each other, and are each independently a substituted or unsubstituted monocyclic or polycyclic aryl group having 6 to 30 carbon atoms.

According to an exemplary embodiment of the present specification, Ar601 and Ar602 are a phenyl group.

According to an exemplary embodiment of the present specification, Chemical Formula ET-1 is represented by the following compound.

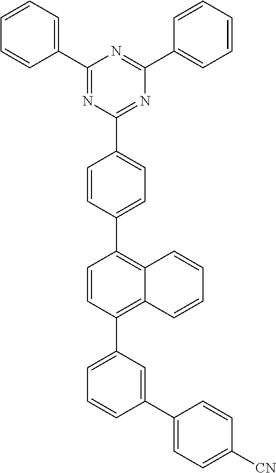 [SMILES: N#Cc1ccc(-c2cccc(-c3ccc(-c4ccc(-c5nc(-c6ccccc6)nc(-c6ccccc6)n5)cc4)c4ccccc34)c2)cc1]

The electron injection layer is a layer which accepts electrons from an electrode. It is preferred that an electron injection material is excellent in ability to transport electrons and has an effect of accepting electrons from the second electrode and an excellent electron injection effect for a light emitting layer or a light emitting material. Further, the electron injection material is preferably a material which prevents excitons produced from a light emitting layer from moving to a hole injection layer and is excellent in ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

Examples of the metal complex compounds include 8-hydroxyquinolinato lithium, bis(8-hydroxyquinolinato) zinc, bis(8-hydroxyquinolinato) copper, bis(8-hydroxyquinolinato) manganese, tris(8-hydroxyquinolinato) aluminum, tris(2-methyl-8-hydroxyquinolinato) aluminum, tris(8-hydroxyquinolinato) gallium, bis(10-hydroxybenzo[h] quinolinato) beryllium, bis(10-hydroxybenzo[h] quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

According to an exemplary embodiment of the present specification, the electron injection and transport layer is a layer that transports electrons to the light emitting layer. For the electron injection and transport layer, materials exemplified for the electron transport layer and the electron injection layer may be used, but the materials are not limited thereto.

According to an exemplary embodiment of the present specification, the electron injection and transport layer may further include a metal complex compound. The metal complex compound is as described above.

The hole adjusting layer is a layer which may improve the service life and efficiency of a device by preventing electrons injected from an electron injection layer from passing through a light emitting layer and entering a hole injection layer. The publicly-known material can be used without limitation, and the hole adjusting layer may be formed between a light emitting layer and a hole injection layer, between a light emitting layer and a hole transport layer, or between a light emitting layer and a layer which simultaneously injects and transports holes.

The electron adjusting layer is a layer which blocks holes from reaching a cathode, and may be generally formed under the same conditions as those of the electron injection layer. Specific examples thereof include oxadiazole derivatives or triazole derivatives, phenanthroline derivatives, aluminum complexes, and the like, but are not limited thereto.

The capping layer is formed to prevent a considerable amount of light from being lost through total reflection of light in the organic light emitting device, and the capping layer has the performance capable of sufficiently protecting the underlying negative electrode and light emitting layer from external moisture penetration or contamination, and has a high refractive index, and thus may prevent light loss caused by total reflection, and materials in the related art can be used without limitation.

According to an exemplary embodiment of the present specification, the capping layer includes a compound represented by the following Chemical Formula CP-1, but is not limited thereto.

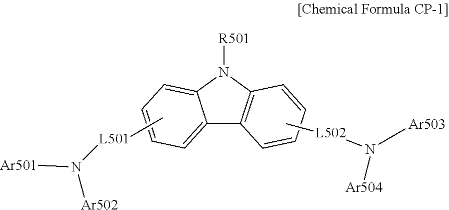

In Chemical Formula CP-1,
L501 and L502 are the same as or different from each other, and are each independently a direct bond; a substituted or unsubstituted arylene group; or a substituted or unsubstituted heteroarylene group, and
R501 and Ar501 to Ar504 are the same as or different from each other, and are each independently a substituted or unsubstituted aryl group; or a substituted or unsubstituted heteroaryl group, or adjacent groups are bonded to each other to form a substituted or unsubstituted ring.

According to an exemplary embodiment of the present specification, L501 and L502 are the same as or different from each other, and are each independently a substituted or unsubstituted monocyclic or polycyclic arylene group having 6 to 30 carbon atoms.

According to an exemplary embodiment of the present specification, L501 and L502 are a phenylene group.

According to an exemplary embodiment of the present specification, R501 and Ar501 to Ar504 are the same as or different from each other, and are each independently a substituted or unsubstituted monocyclic or polycyclic aryl group having 6 to 30 carbon atoms, or are bonded to an adjacent group to form a substituted or unsubstituted monocyclic or polycyclic hetero ring having 2 to 30 carbon atoms.

According to an exemplary embodiment of the present specification, R501 and Ar501 to Ar504 are a phenyl group, or are bonded to an adjacent group to form a carbazole which is unsubstituted or substituted with a phenyl group.

According to an exemplary embodiment of the present specification, Ar501 and L501 are bonded to form a carbazole substituted with a phenyl group.

According to an exemplary embodiment of the present specification, Ar503 is bonded to L503 to form a carbazole substituted with a phenyl group.

According to an exemplary embodiment of the present specification, Chemical Formula CP-1 is represented by the following compound.

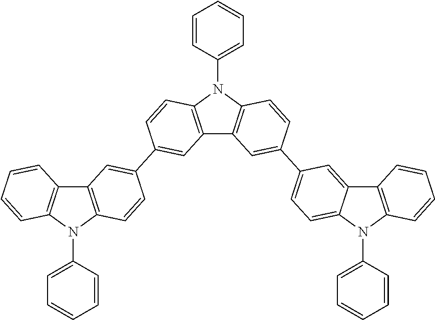 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

The organic light emitting device according to the present specification may be a top-emission type, a bottom-emission type or a dual-emission type depending on the materials used.

The organic light emitting device according to the present specification may be included and used in various electronic devices. For example, the electronic device may be a display panel, a touch panel, a solar module, a lighting device, and the like, and is not limited thereto.

Hereinafter, the present specification will be described in detail with reference to Examples for specifically describing the present specification. However, the Examples according to the present specification may be modified in various forms, and it is not interpreted that the scope of the present application is limited to the Examples described in detail below. The Examples of the present application are provided to explain the present specification more completely to a person with ordinary skill in the art.

### Preparation Example 1 (Synthesis of Chemical Formula 1)

### 1) Synthesis of Chemical Formula A1

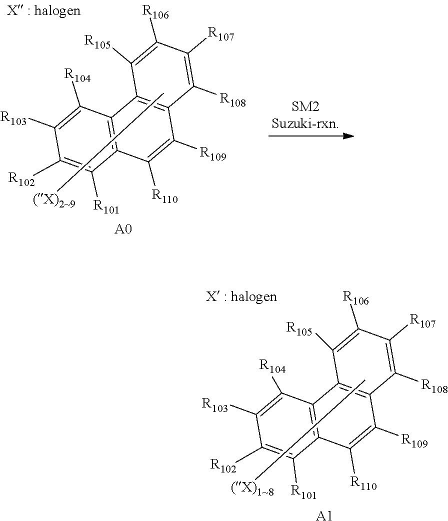

After SM1 (AG, 1 eq) and SM2 (1.1 eq) were added to tetrahydrofuran (THF) (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formula A1 (A1-1 to A1-5).

Compounds A1-1 to A1-5 in [Table 1] were synthesized in the same manner as in the method of synthesizing Chemical Formula A1, except that SM1 and SM2 were changed to the materials in the following Table 1.

TABLE 1

| | | | | | MS |
|---|---|---|---|---|---|
| | SM1 | SM2 | Product | Yield | [M + H]+ |
| A1-1 | 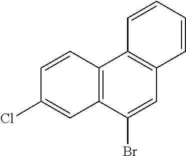 | 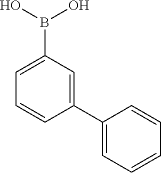 | 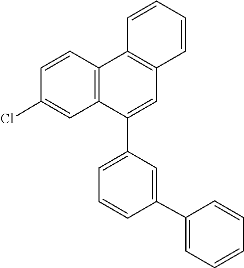 [SMILES: Clc1ccc2c(c1)c(-c1cccc(-c3ccccc3)c1)cc1ccccc12] | 73% | 365.87 |
| A1-2 | 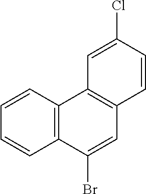 | 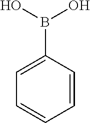 | 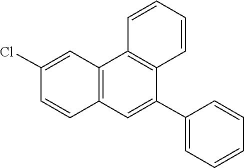 [SMILES: Clc1ccc2cc(-c3ccccc3)c3ccccc3c2c1] | 75% | 289.77 |
| A1-3 | 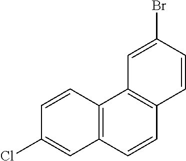 | 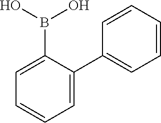 | 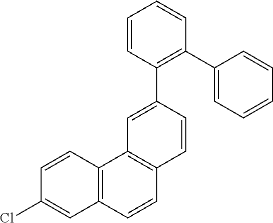 [SMILES: Clc1ccc2c(ccc3ccc(-c4ccccc4-c4ccccc4)cc32)c1] | 70% | 365.87 |
| A1-4 | 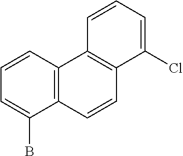 | 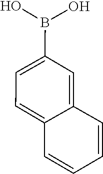 | 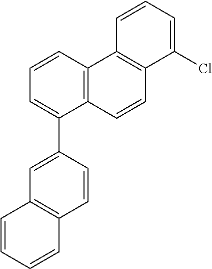 [SMILES: Clc1cccc2c1ccc1c(-c3ccc4ccccc4c3)cccc12] | 71% | 339.83 |
| A1-5 | 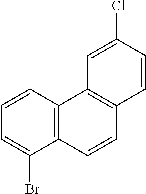 | 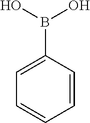 | 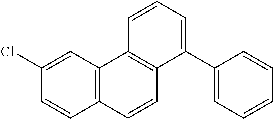 [SMILES: Clc1ccc2ccc3c(-c4ccccc4)cccc3c2c1] | 69% | 289.77 |

### 2) Synthesis of Chemical Formulae A2 and B2

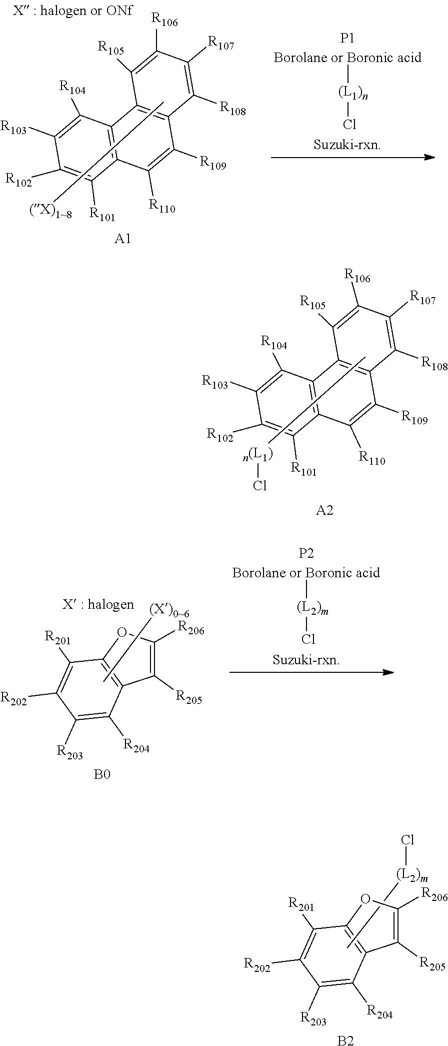

After SM1 (one of A1 or BO, 1 eq) and SM2 (one of P1 or P2, 1.1 eq) were added to tetrahydrofuran (THF) (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formulae A2 and B2 (A2-1 to A2-3 and B2-1 to B2-5).

A2-1 to A2-3 in [Table 2] and B2-1 to B2-5 in [Table 3] were synthesized in the same manner as in the method of synthesizing Chemical Formulae A2 and B2, except that SM1 and SM2 were changed to the materials in the following Tables 2 and 3.

TABLE 2

| | | | | | MS |
|---|---|---|---|---|---|
| | SM1 | SM2 | Product | Yield | [M + H]+ |
| A2-1 | 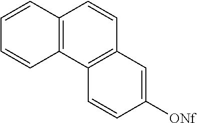 | 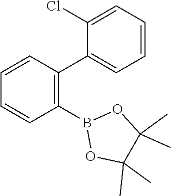 | 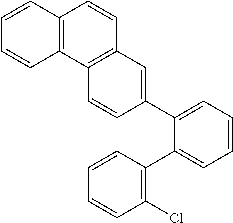 [SMILES: Clc1ccccc1-c1ccccc1-c1ccc2c(ccc3ccccc32)c1] | 73% | 365.87 |
| A2-2 | 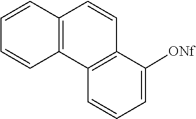 | 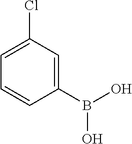 | 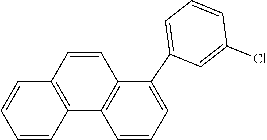 [SMILES: Clc1cccc(-c2cccc3c2ccc2ccccc23)c1] | 72% | 289.77 |
| A2-3 | 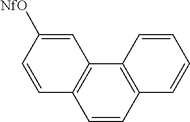 | 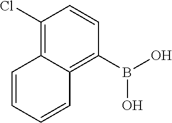 | 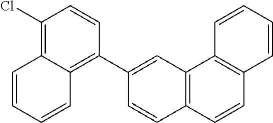 [SMILES: Clc1ccc(-c2ccc3ccc4ccccc4c3c2)c2ccccc12] | 70% | 339.83 |

TABLE 3

| | | | | | MS |
|---|---|---|---|---|---|
| | SM1 | SM2 | Product | Yield | [M + H]+ |
| B2-1 | 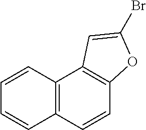 | 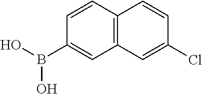 | 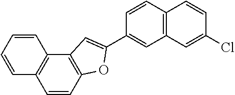 [SMILES: Clc1ccc2ccc(-c3cc4c(ccc5ccccc54)o3)cc2c1] | 69% | 329.80 |
| B2-2 | 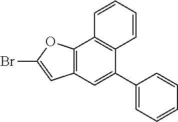 | 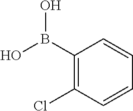 | 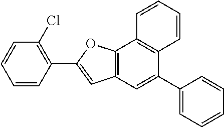 [SMILES: Clc1ccccc1-c1cc2cc(-c3ccccc3)c3ccccc3c2o1] | 71% | 355.83 |
| B2-3 | 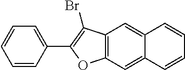 | 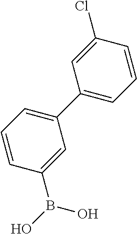 | 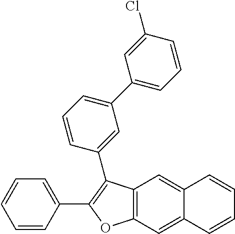 [SMILES: Clc1cccc(-c2cccc(-c3c(-c4ccccc4)oc4cc5ccccc5cc34)c2)c1] | 68% | 431.93 |
| B2-4 | 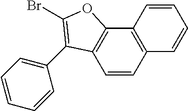 | 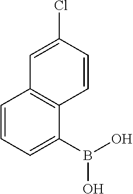 | 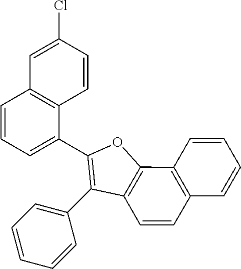 [SMILES: Clc1ccc2c(-c3oc4c(ccc5ccccc54)c3-c3ccccc3)cccc2c1] | 65 % | 405.89 |
| B2-5 | 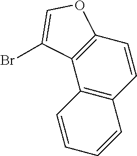 | 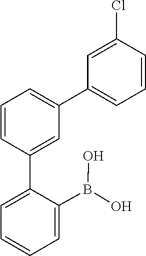 | 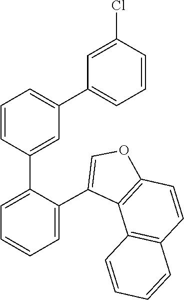 [SMILES: Clc1cccc(-c2cccc(-c3ccccc3-c3coc4ccc5ccccc5c34)c2)c1] | 67% | 431.93 |

### 3) Synthesis of Chemical Formulae A3 and B3

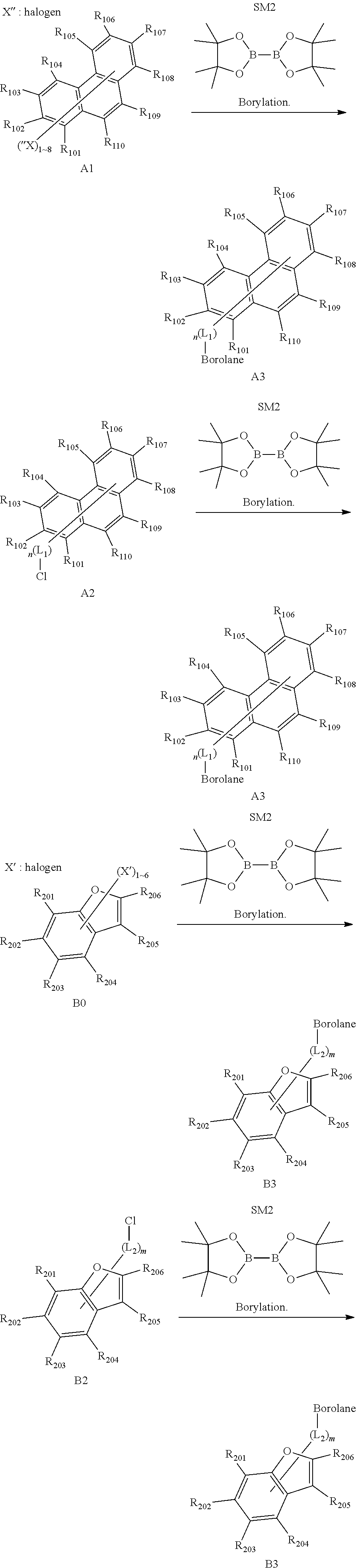

SM1 (one of A1, A2, BO, and B2, 1 eq) and SM2 (1.3 eq) were put into 1,4-dioxane (12-fold <mass ratio> compared to SM1), and potassium acetate (3 eq) was added thereto, and the resulting mixture was stirred and refluxed. After bis(diphenylphosphino)ferrocene dichloropalladium (0.05 eq) was stirred in 1,4-dioxane for 5 minutes, the resulting mixture was added thereto, and 2 hours later, the mixture was cooled to room temperature after confirming that the reaction was terminated. After ethanol and water were added thereto, the resulting mixture was filtered and purified by recrystallization with ethyl acetate and ethanol to prepare Chemical Formulae A3 and B3 (A3-1 to A3-8 and B3-1 to B3-31).

A3-1 to A3-8 in [Table 4] and B3-1 to B3-31 in [Table 5] were synthesized in the same manner as in the method of synthesizing Chemical Formulae A3 and B3, except that SM1 and SM2 were changed to the materials in the following Tables 4 and 5.

TABLE 4

| | | | | | MS |
|---|---|---|---|---|---|
| | SM1 | SM2 | Product | Yield | [M + H]+ |
| A3-1 | A1-1 | 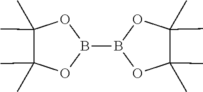 | 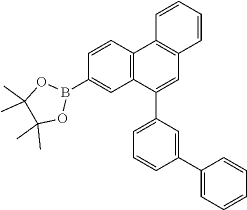 [SMILES: CC1(C)OB(c2ccc3c(c2)c(-c2cccc(-c4ccccc4)c2)cc2ccccc23)OC1(C)C] | 75% | 457.39 |
| A3-2 | A1-2 | 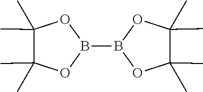 | 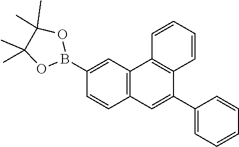 [SMILES: CC1(C)OB(c2ccc3cc(-c4ccccc4)c4ccccc4c3c2)OC1(C)C] | 70% | 381.29 |
| A3-3 | A1-3 | 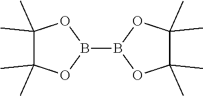 | 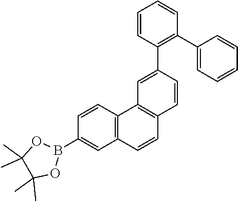 [SMILES: CC1(C)OB(c2ccc3c(ccc4ccc(-c5ccccc5-c5ccccc5)cc43)c2)OC1(C)C] | 76% | 457.39 |
| A3-4 | A1-4 | 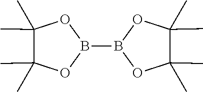 | 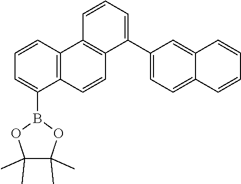 [SMILES: CC1(C)OB(c2cccc3c2ccc2c(-c4ccc5ccccc5c4)cccc23)OC1(C)C] | 73% | 431.35 |
| A3-5 | A2-1 | 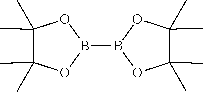 | 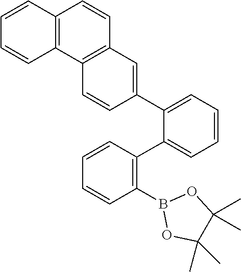 [SMILES: CC1(C)OB(c2ccccc2-c2ccccc2-c2ccc3c(ccc4ccccc43)c2)OC1(C)C] | 74% | 457.39 |
| A3-6 | A2-2 | 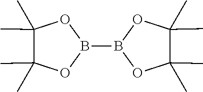 | 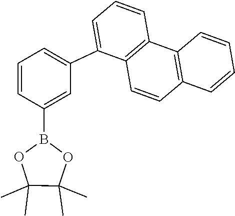 [SMILES: CC1(C)OB(c2cccc(-c3cccc4c3ccc3ccccc34)c2)OC1(C)C] | 71% | 381.29 |
| A3-7 | A2-3 | 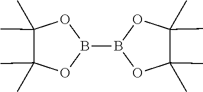 | 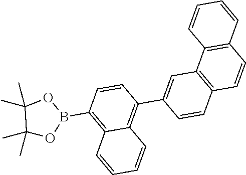 [SMILES: CC1(C)OB(c2ccc(-c3ccc4ccc5ccccc5c4c3)c3ccccc23)OC1(C)C] | 70% | 431.35 |
| A3-8 | A1-5 | 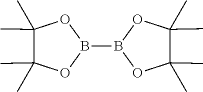 | 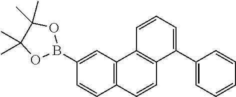 [SMILES: CC1(C)OB(c2ccc3ccc4c(-c5ccccc5)cccc4c3c2)OC1(C)C] | 73% | 381.29 |

TABLE 5

| | | | | | MS |
|---|---|---|---|---|---|
| | SM1 | SM2 | Product | Yield | [M + H]+ |
| B3-1 | 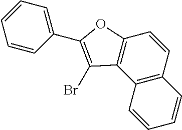 | 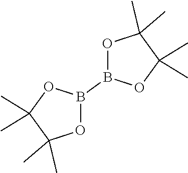 | 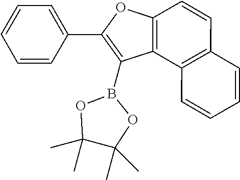 [SMILES: CC1(C)OB(c2c(-c3ccccc3)oc3ccc4ccccc4c23)OC1(C)C] | 76% | 371.26 |
| B3-2 | 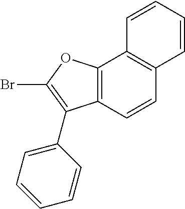 | 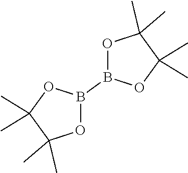 | 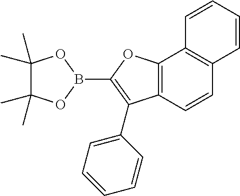 [SMILES: CC1(C)OB(c2oc3c(ccc4ccccc43)c2-c2ccccc2)OC1(C)C] | 71% | 371.26 |
| B3-3 | 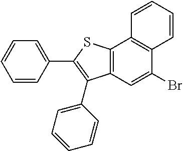 | 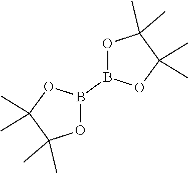 | 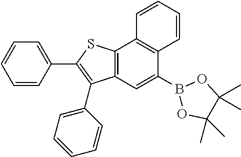 [SMILES: CC1(C)OB(c2cc3c(-c4ccccc4)c(-c4ccccc4)sc3c3ccccc23)OC1(C)C] | 73% | 463.41 |
| B3-4 | 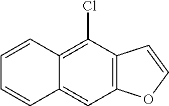 | 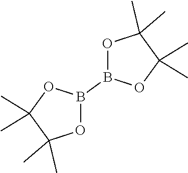 | 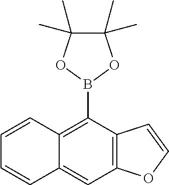 [SMILES: CC1(C)OB(c2c3ccccc3cc3occc23)OC1(C)C] | 70% | 295.16 |
| B3-5 | 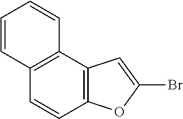 | 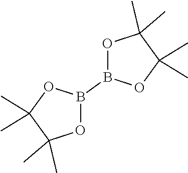 | 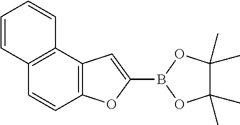 [SMILES: CC1(C)OB(c2cc3c(ccc4ccccc43)o2)OC1(C)C] | 75% | 295.16 |
| B3-6 | 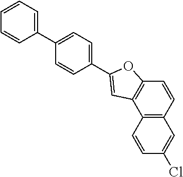 | 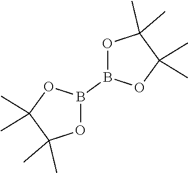 | 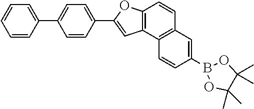 [SMILES: CC1(C)OB(c2ccc3c(ccc4oc(-c5ccc(-c6ccccc6)cc5)cc43)c2)OC1(C)C] | 69% | 447.35 |
| B3-7 | 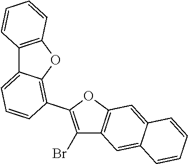 | 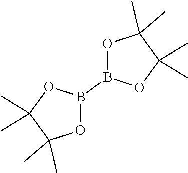 | 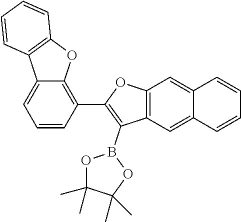 [SMILES: CC1(C)OB(c2c(-c3cccc4c3oc3ccccc34)oc3cc4ccccc4cc23)OC1(C)C] | 73% | 461.34 |
| B3-8 | 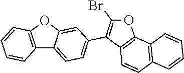 | 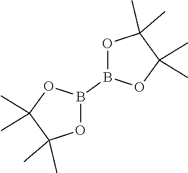 | 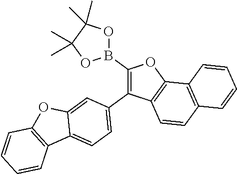 [SMILES: CC1(C)OB(c2oc3c(ccc4ccccc43)c2-c2ccc3c(c2)oc2ccccc23)OC1(C)C] | 71% | 461.34 |
| B3-9 | 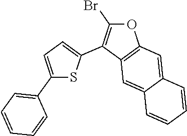 | 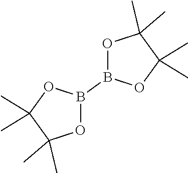 | 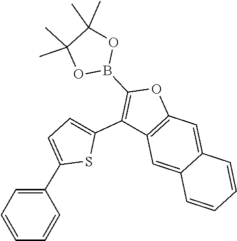 [SMILES: CC1(C)OB(c2oc3cc4ccccc4cc3c2-c2ccc(-c3ccccc3)s2)OC1(C)C] | 70% | 453.38 |
| B3-10 | 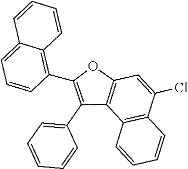 | 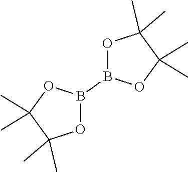 | 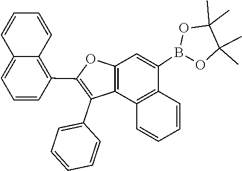 [SMILES: CC1(C)OB(c2cc3oc(-c4cccc5ccccc45)c(-c4ccccc4)c3c3ccccc23)OC1(C)C] | 75% | 497.41 |
| B3-11 | 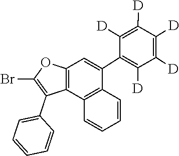 | 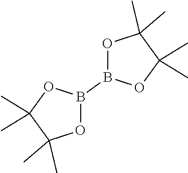 | 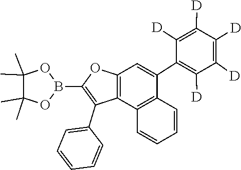 [SMILES: [2H]c1c([2H])c([2H])c(-c2cc3oc(B4OC(C)(C)C(C)(C)O4)c(-c4ccccc4)c3c3ccccc23)c([2H])c1[2H]] | 73% | 452.38 |
| B3-12 | 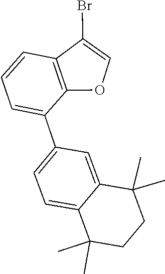 | 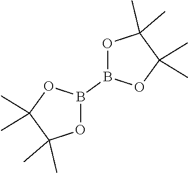 | 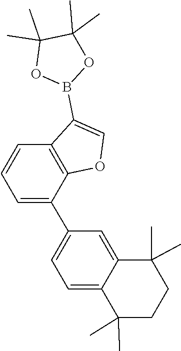 [SMILES: CC1(C)CCC(C)(C)c2cc(-c3cccc4c(B5OC(C)(C)C(C)(C)O5)coc34)ccc21] | 72% | 431.30 |
| B3-13 | 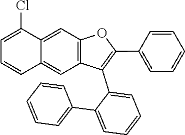 | 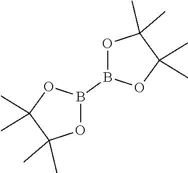 | 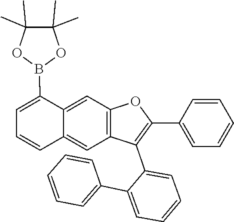 [SMILES: CC1(C)OB(c2cccc3cc4c(-c5ccccc5-c5ccccc5)c(-c5ccccc5)oc4cc23)OC1(C)C] | 70% | 523.45 |
| B3-14 | 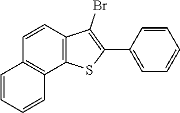 | 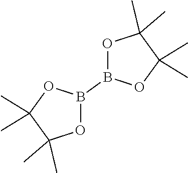 | 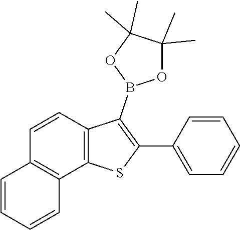 [SMILES: CC1(C)OB(c2c(-c3ccccc3)sc3c2ccc2ccccc23)OC1(C)C] | 68% | 387.32 |
| B3-15 | 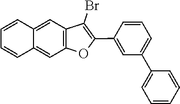 | 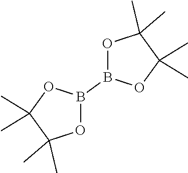 | 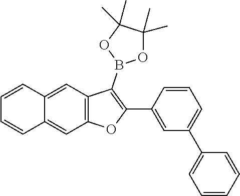 [SMILES: CC1(C)OB(c2c(-c3cccc(-c4ccccc4)c3)oc3cc4ccccc4cc23)OC1(C)C] | 73% | 447.35 |
| B3-16 | 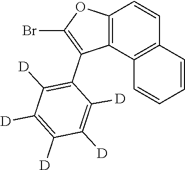 | 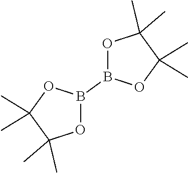 | 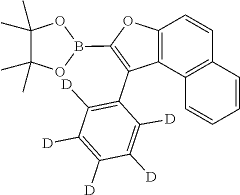 [SMILES: [2H]c1c([2H])c([2H])c(-c2c(B3OC(C)(C)C(C)(C)O3)oc3ccc4ccccc4c23)c([2H])c1[2H]] | 70% | 376.29 |
| B3-17 | 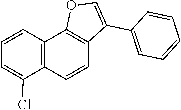 | 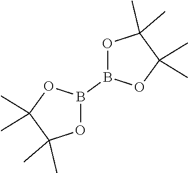 | 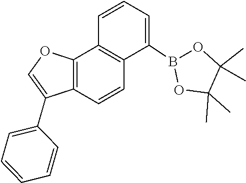 [SMILES: CC1(C)OB(c2cccc3c2ccc2c(-c4ccccc4)coc23)OC1(C)C] | 71% | 371.26 |
| B3-18 | B2-1 | 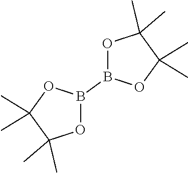 | 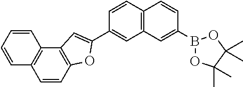 [SMILES: CC1(C)OB(c2ccc3ccc(-c4cc5c(ccc6ccccc65)o4)cc3c2)OC1(C)C] | 68% | 421.32 |
| B3-19 | 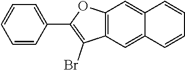 | 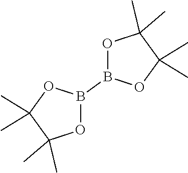 | 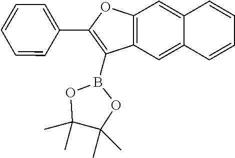 [SMILES: CC1(C)OB(c2c(-c3ccccc3)oc3cc4ccccc4cc23)OC1(C)C] | 71% | 371.26 |
| B3-20 | 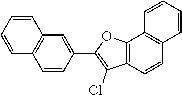 | 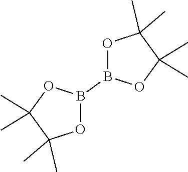 | 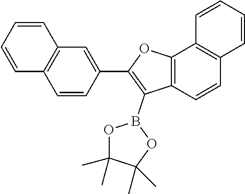 [SMILES: CC1(C)OB(c2c(-c3ccc4ccccc4c3)oc3c2ccc2ccccc23)OC1(C)C] | 69% | 421.32 |
| B3-21 | 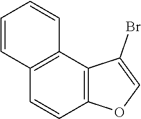 | 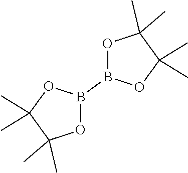 | 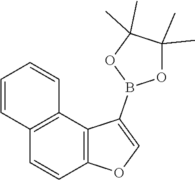 [SMILES: CC1(C)OB(c2coc3ccc4ccccc4c23)OC1(C)C] | 71% | 295.16 |
| B3-22 | B2-2 | 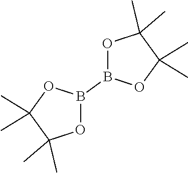 | 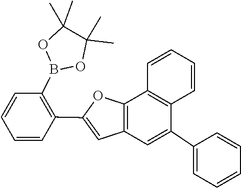 [SMILES: CC1(C)OB(c2ccccc2-c2cc3cc(-c4ccccc4)c4ccccc4c3o2)OC1(C)C] | 73% | 447.35 |
| B3-23 | B2-3 | 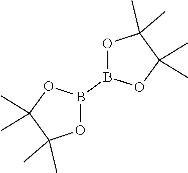 | 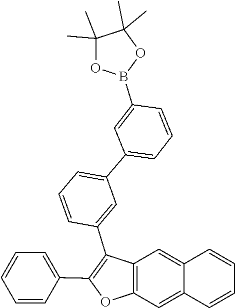 [SMILES: CC1(C)OB(c2cccc(-c3cccc(-c4c(-c5ccccc5)oc5cc6ccccc6cc45)c3)c2)OC1(C)C] | 75% | 523.45 |
| B3-24 | B2-4 | 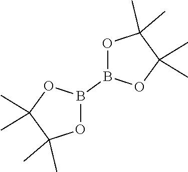 | 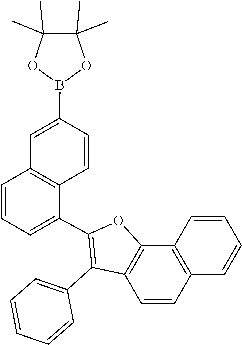 [SMILES: CC1(C)OB(c2ccc3c(-c4oc5c(ccc6ccccc65)c4-c4ccccc4)cccc3c2)OC1(C)C] | 73% | 497.41 |
| B3-25 | B2-5 | 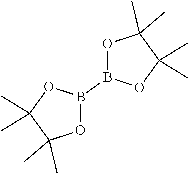 | 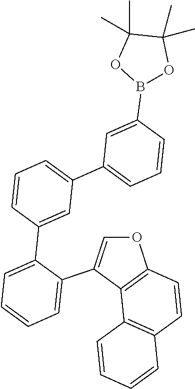 [SMILES: CC1(C)OB(c2cccc(-c3cccc(-c4ccccc4-c4coc5ccc6ccccc6c45)c3)c2)OC1(C)C] | 71% | 523.45 |
| B3-26 | 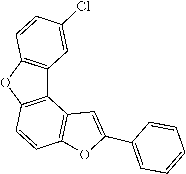 | 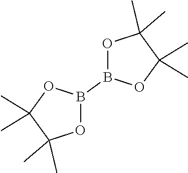 | 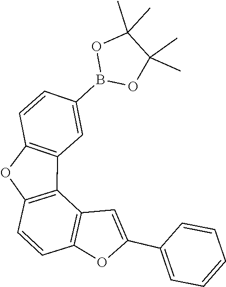 [SMILES: CC1(C)OB(c2ccc3oc4ccc5oc(-c6ccccc6)cc5c4c3c2)OC1(C)C] | 70% | 411.28 |
| B3-27 | 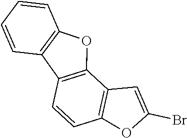 | 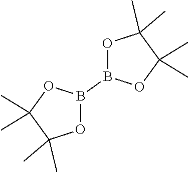 | 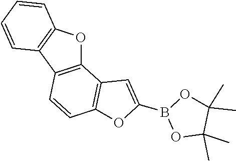 [SMILES: CC1(C)OB(c2cc3c(ccc4c5ccccc5oc34)o2)OC1(C)C] | 73% | 335.18 |
| B3-28 | 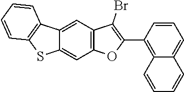 | 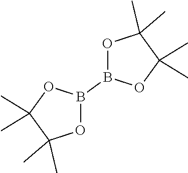 | 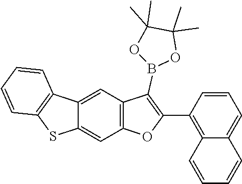 [SMILES: CC1(C)OB(c2c(-c3cccc4ccccc34)oc3cc4sc5ccccc5c4cc23)OC1(C)C] | 72% | 477.40 |
| B3-29 | 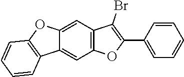 | 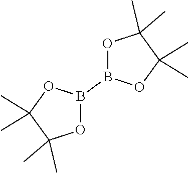 | 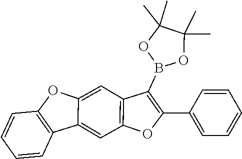 [SMILES: CC1(C)OB(c2c(-c3ccccc3)oc3cc4c(cc23)oc2ccccc24)OC1(C)C] | 70% | 411.28 |
| B3-30 | 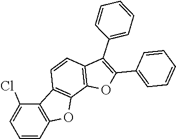 | 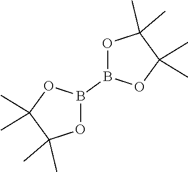 | 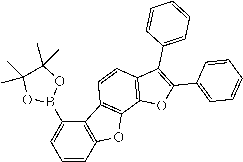 [SMILES: CC1(C)OB(c2cccc3oc4c(ccc5c(-c6ccccc6)c(-c6ccccc6)oc54)c23)OC1(C)C] | 68% | 487.37 |
| B3-31 | 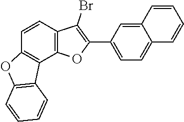 | 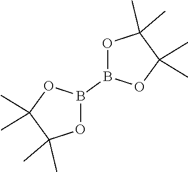 | 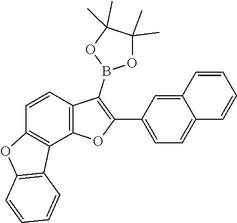 [SMILES: CC1(C)OB(c2c(-c3ccc4ccccc4c3)oc3c2ccc2oc4ccccc4c23)OC1(C)C] | 65% | 461.34 |

In the synthesis of Chemical Formula B3, B3-1 to B3-25 were synthesized with reference to LGC KR 2024-0003039 A, KR 2024-0003040 A, and KR 2024-0002594 A, and B3-26 to B3-31 were synthesized with reference to LGC KR 2024-0007056 A.

### 4) Synthesis of Int 1.

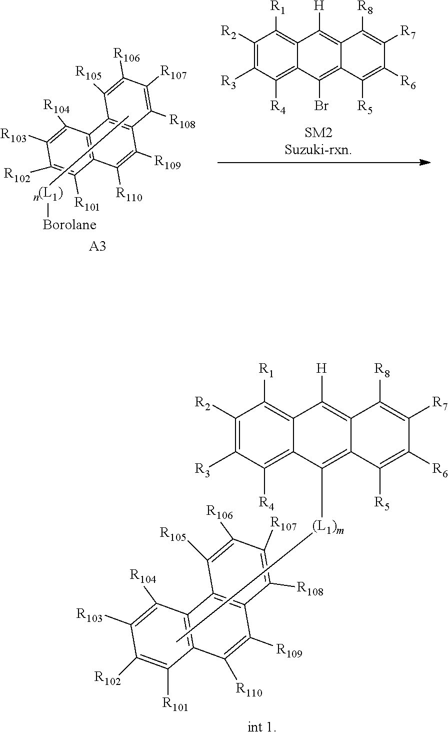

After SM1 (one of A3, 1 eq) and SM2 (1 eq) were added to tetrahydrofuran (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was recrystallized with chloroform and ethyl acetate to prepare Chemical Formula int. (int 1-1. to int 1-14.) above.

int 1-1. to int 1-14. in [Table 6] were synthesized in the same manner as in the method of synthesizing Chemical Formula int 1., except that SM1 and SM2 were changed to the materials in the following Table 6.

TABLE 6

| | | | | | MS |
|---|---|---|---|---|---|
| | SM2 | SM1 | Product | Yield | [M + H]+ |
| int 1-1 | 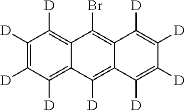 | 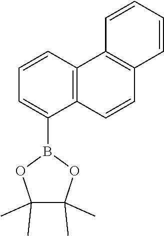 | 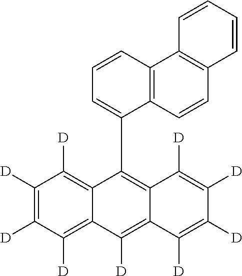 [SMILES: [2H]c1c([2H])c([2H])c2c(-c3cccc4c3ccc3ccccc34)c3c([2H])c([2H])c([2H])c([2H])c3c([2H])c2c1[2H]] | 73% | 364.51 |
| int 1-2 | 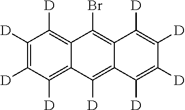 | 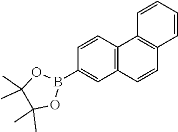 | 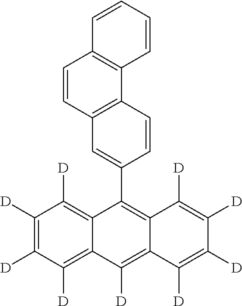 [SMILES: [2H]c1c([2H])c([2H])c2c(-c3ccc4c(ccc5ccccc54)c3)c3c([2H])c([2H])c([2H])c([2H])c3c([2H])c2c1[2H]] | 75% | 364.51 |
| int 1-3 | 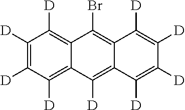 | 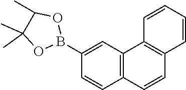 | 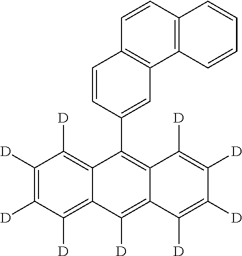 [SMILES: [2H]c1c([2H])c([2H])c2c(-c3ccc4ccc5ccccc5c4c3)c3c([2H])c([2H])c([2H])c([2H])c3c([2H])c2c1[2H]] | 70% | 364.51 |
| int 1-4 | 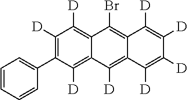 | 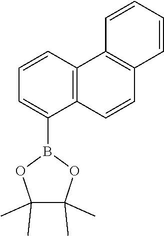 | 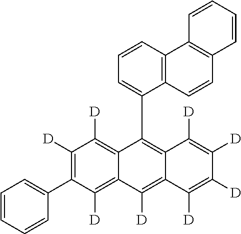 [SMILES: [2H]c1c([2H])c([2H])c2c(-c3cccc4c3ccc3ccccc34)c3c([2H])c([2H])c(-c4ccccc4)c([2H])c3c([2H])c2c1[2H]] | 71% | 439.60 |
| int 1-5 | 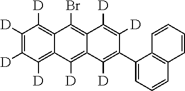 | 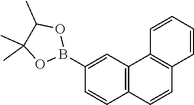 | 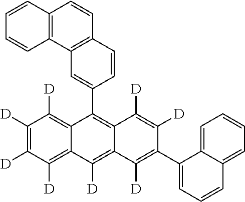 [SMILES: [2H]c1c([2H])c([2H])c2c(-c3ccc4ccc5ccccc5c4c3)c3c([2H])c([2H])c(-c4cccc5ccccc45)c([2H])c3c([2H])c2c1[2H]] | 72% | 489.66 |
| int 1-6 | 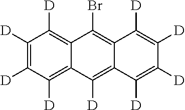 | 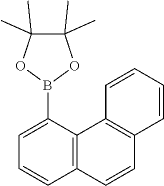 | 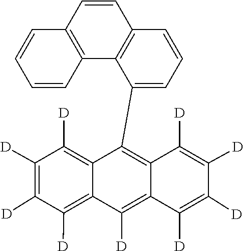 [SMILES: [2H]c1c([2H])c([2H])c2c(-c3cccc4ccc5ccccc5c34)c3c([2H])c([2H])c([2H])c([2H])c3c([2H])c2c1[2H]] | 73% | 364.51 |
| int 1-7 | 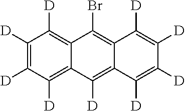 | A3-1 | 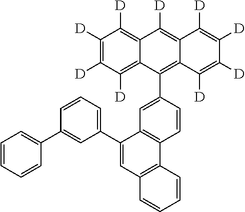 [SMILES: [2H]c1c([2H])c([2H])c2c(-c3ccc4c(c3)c(-c3cccc(-c5ccccc5)c3)cc3ccccc34)c3c([2H])c([2H])c([2H])c([2H])c3c([2H])c2c1[2H]] | 71% | 516.70 |
| int 1-8 | 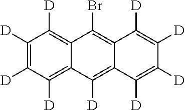 | A3-2 | 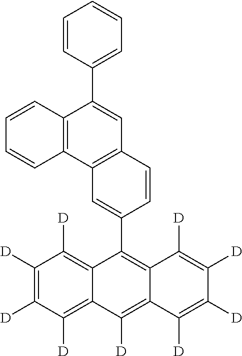 [SMILES: [2H]c1c([2H])c([2H])c2c(-c3ccc4cc(-c5ccccc5)c5ccccc5c4c3)c3c([2H])c([2H])c([2H])c([2H])c3c([2H])c2c1[2H]] | 70% | 440.60 |
| int 1-9 | 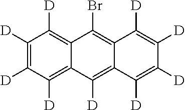 | A3-3 | 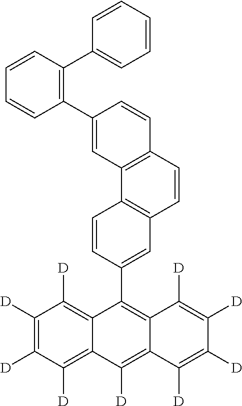 [SMILES: [2H]c1c([2H])c([2H])c2c(-c3ccc4c(ccc5ccc(-c6ccccc6-c6ccccc6)cc54)c3)c3c([2H])c([2H])c([2H])c([2H])c3c([2H])c2c1[2H]] | 75% | 516.70 |
| int 1-10 | 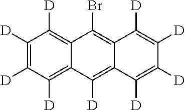 | A3-4 | 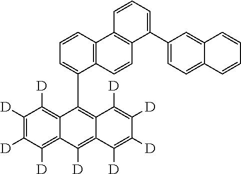 [SMILES: [2H]c1c([2H])c([2H])c2c(-c3cccc4c3ccc3c(-c5ccc6ccccc6c5)cccc34)c3c([2H])c([2H])c([2H])c([2H])c3c([2H])c2c1[2H]] | 73% | 490.66 |
| int 1-11 | 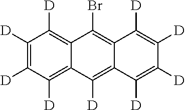 | A3-5 | 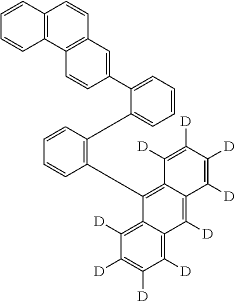 [SMILES: [2H]c1c([2H])c([2H])c2c(-c3ccccc3-c3ccccc3-c3ccc4c(ccc5ccccc54)c3)c3c([2H])c([2H])c([2H])c([2H])c3c([2H])c2c1[2H]] | 71% | 516.70 |
| int 1-12 | 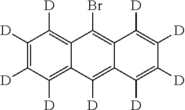 | A3-6 | 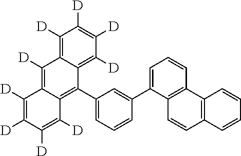 [SMILES: [2H]c1c([2H])c([2H])c2c(-c3cccc(-c4cccc5c4ccc4ccccc45)c3)c3c([2H])c([2H])c([2H])c([2H])c3c([2H])c2c1[2H]] | 75% | 440.60 |
| int 1-13 | 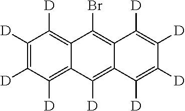 | A3-7 | 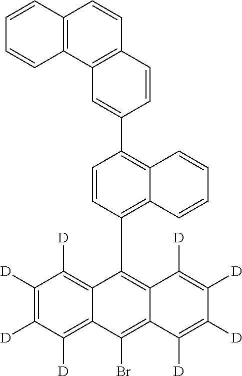 [SMILES: [2H]c1c([2H])c([2H])c2c(-c3ccc(-c4ccc5ccc6ccccc6c5c4)c4ccccc34)c3c([2H])c([2H])c([2H])c([2H])c3c(Br)c2c1[2H]] | 76% | 490.66 |
| int 1-14 | 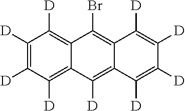 | A3-8 | 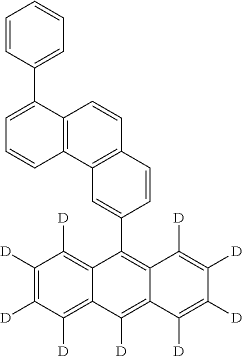 [SMILES: [2H]c1c([2H])c([2H])c2c(-c3ccc4ccc5c(-c6ccccc6)cccc5c4c3)c3c([2H])c([2H])c([2H])c([2H])c3c([2H])c2c1[2H]] | 70% | 440.60 |

### 5) Synthesis of Int 2.

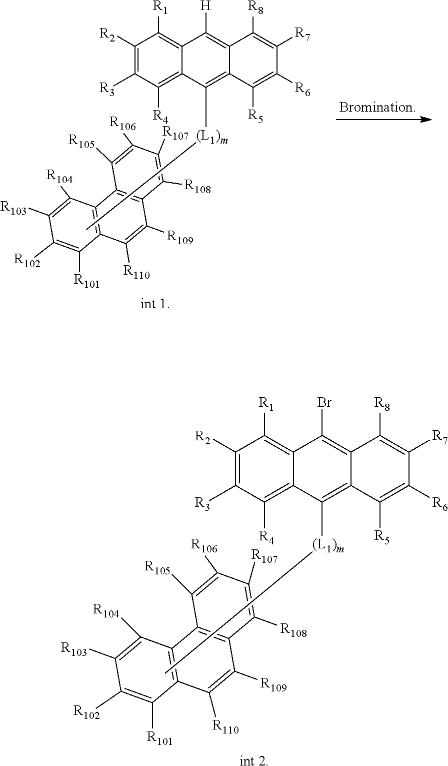

After SM1 (one of int 1., 1 eq) was dissolved in chloroform (excess), the temperature was lowered to 0° C. and stabilized, and then N-bromosuccinimide (NBS) (1 eq) was dissolved in dimethylformamide (DMF) (excess), and the resulting solution was added dropwise thereto. Thereafter, the reactant was warmed to room temperature and stirred for 1 hour, and 1 N HCl (excess) was added thereto to terminate the reaction. After the reaction was completed, the solvent was removed by separating layers, and then the residue was subjected to silica column chromatography (ethyl acetate/hexane 1:15) to prepare Chemical Formula int 2. (int 2-1. to int 2-14.).

int 2-1. to int 2-14. in [Table 7] were synthesized in the same manner as in the method of synthesizing Chemical Formula int 2., except that SM1 and SM2 were changed to the materials in the following Table 7.

TABLE 7

| | | | | | MS |
|---|---|---|---|---|---|
| | SM1 | SM2 | Product | Yield | [M + H]+ |
| int 2-1 | int 1-1 | NBS (1 eq) chloroform, DMF | 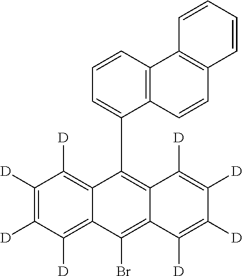 [SMILES: [2H]c1c([2H])c([2H])c2c(-c3cccc4c3ccc3ccccc34)c3c([2H])c([2H])c([2H])c([2H])c3c(Br)c2c1[2H]] | 73% | 442.40 |
| int 2-2 | int 1-2 | NBS (1 eq) chloroform, DMF | 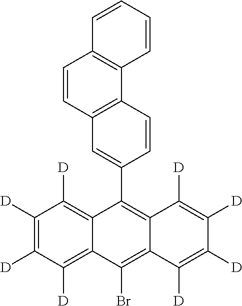 [SMILES: [2H]c1c([2H])c([2H])c2c(-c3ccc4c(ccc5ccccc54)c3)c3c([2H])c([2H])c([2H])c([2H])c3c(Br)c2c1[2H]] | 75% | 442.40 |
| int 2-3 | int 1-3 | NBS (1 eq) chloroform, DMF | 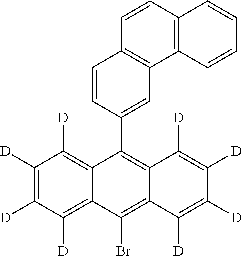 [SMILES: [2H]c1c([2H])c([2H])c2c(-c3ccc4ccc5ccccc5c4c3)c3c([2H])c([2H])c([2H])c([2H])c3c(Br)c2c1[2H]] | 76% | 442.40 |
| int 2-4 | int 1-4 | NBS (1 eq) chloroform, DMF | 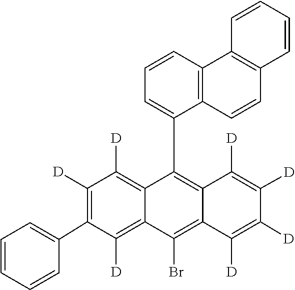 [SMILES: [2H]c1c([2H])c([2H])c2c(-c3cccc4c3ccc3ccccc34)c3c([2H])c([2H])c(-c4ccccc4)c([2H])c3c(Br)c2c1[2H]] | 78% | 517.49 |
| int 2-5 | int 1-5 | NBS (1 eq) chloroform, DME | 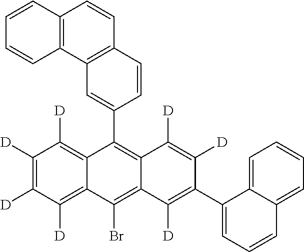 [SMILES: [2H]c1c([2H])c([2H])c2c(-c3ccc4ccc5ccccc5c4c3)c3c([2H])c([2H])c(-c4cccc5ccccc45)c([2H])c3c(Br)c2c1[2H]] | 71% | 567.55 |
| int 2-6 | int 1-6 | NBS (1 eq) chloroform, DME | 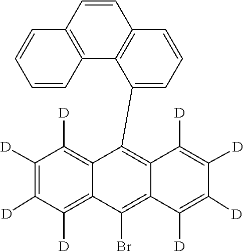 [SMILES: [2H]c1c([2H])c([2H])c2c(-c3cccc4ccc5ccccc5c34)c3c([2H])c([2H])c([2H])c([2H])c3c(Br)c2c1[2H]] | 74% | 442.40 |
| int 2-7 | int 1-7 | NBS (1 eq) chloroform, DME | 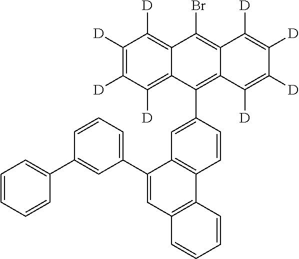 [SMILES: [2H]c1c([2H])c([2H])c2c(-c3ccc4c(c3)c(-c3cccc(-c5ccccc5)c3)cc3ccccc34)c3c([2H])c([2H])c([2H])c([2H])c3c(Br)c2c1[2H]] | 78% | 594.59 |
| int 2-8 | int 1-8 | NBS (1 eq) chloroform, DMF | 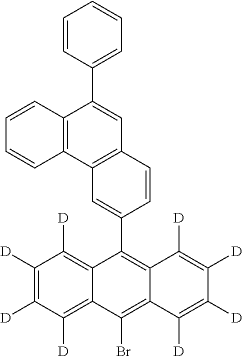 [SMILES: [2H]c1c([2H])c([2H])c2c(-c3ccc4cc(-c5ccccc5)c5ccccc5c4c3)c3c([2H])c([2H])c([2H])c([2H])c3c(Br)c2c1[2H]] | 79% | 518.49 |
| int 2-9 | int 1-9 | NBS (1 eq) chloroform, DMF | 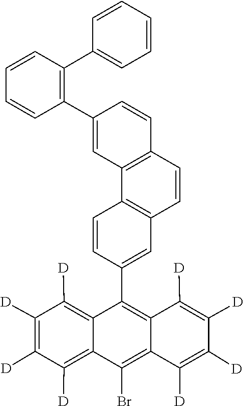 [SMILES: [2H]c1c([2H])c([2H])c2c(-c3ccc4c(ccc5ccc(-c6ccccc6-c6ccccc6)cc54)c3)c3c([2H])c([2H])c([2H])c([2H])c3c(Br)c2c1[2H]] | 80% | 594.59 |
| int 2-10 | int 1-10 | NBS (1 eq) chloroform, DME | 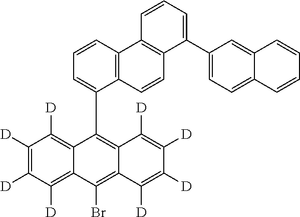 [SMILES: [2H]c1c([2H])c([2H])c2c(-c3cccc4c3ccc3c(-c5ccc6ccccc6c5)cccc34)c3c([2H])c([2H])c([2H])c([2H])c3c(Br)c2c1[2H]] | 74% | 568.55 |
| int 2-11 | int 1-11 | NBS (1 eq) chloroform, DME | 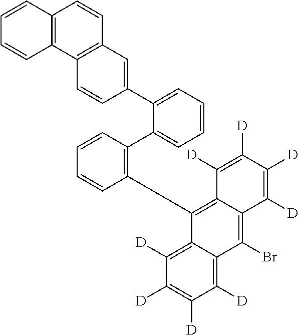 [SMILES: [2H]c1c([2H])c([2H])c2c(-c3ccccc3-c3ccccc3-c3ccc4c(ccc5ccccc54)c3)c3c([2H])c([2H])c([2H])c([2H])c3c(Br)c2c1[2H]] | 73% | 594.59 |
| int 2-12 | int 1-12 | NBS (1 eq) chloroform, DMF | 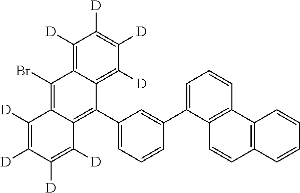 [SMILES: [2H]c1c([2H])c([2H])c2c(-c3cccc(-c4cccc5c4ccc4ccccc45)c3)c3c([2H])c([2H])c([2H])c([2H])c3c(Br)c2c1[2H]] | 71% | 518.49 |
| int 2-13 | int 1-13 | NBS (1 eq) chloroform, DME | 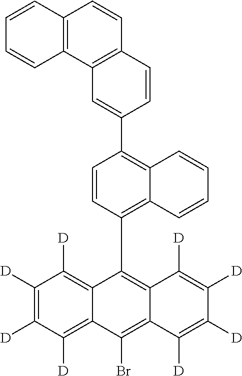 [SMILES: [2H]c1c([2H])c([2H])c2c(-c3ccc(-c4ccc5ccc6ccccc6c5c4)c4ccccc34)c3c([2H])c([2H])c([2H])c([2H])c3c(Br)c2c1[2H]] | 76% | 568.55 |
| int 2-14 | int 1-14 | NBS (1 eq) chloroform, DMF | 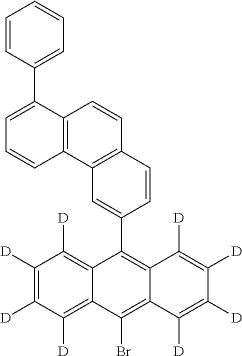 [SMILES: [2H]c1c([2H])c([2H])c2c(-c3ccc4ccc5c(-c6ccccc6)cccc5c4c3)c3c([2H])c([2H])c([2H])c([2H])c3c(Br)c2c1[2H]] | 75% | 518.49 |

### 6) Synthesis of Chemical Formula 1

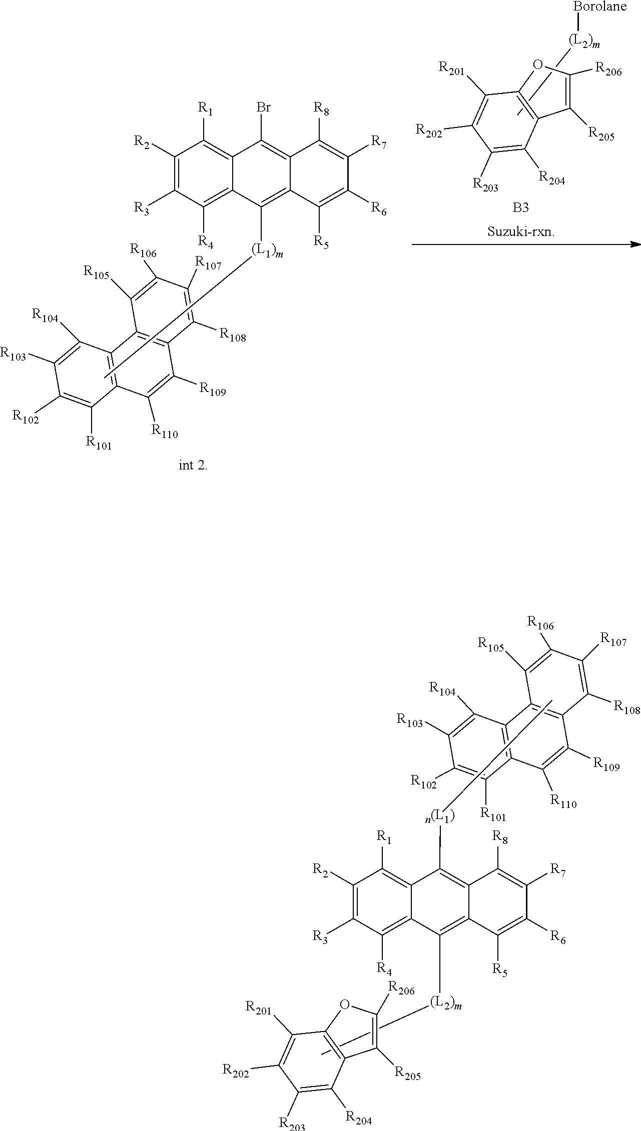

After SM1 (one of int 2., 1 eq) and SM2 (1.1 eq) were added to tetrahydrofuran (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formula 1 (Compounds 1 to 31) above.

Compounds 1 to 31 in [Table 8] were synthesized in the same manner as in the method of synthesizing Chemical Formula int., except that SM1 and SM2 were changed to the materials in the following Table 8.

TABLE 8

| Com- | | | | | MS |
|---|---|---|---|---|---|
| pound | SM1 | SM2 | Product | Yield | [M + H]+ |
| 1 | int 2-1 | B3-1 | 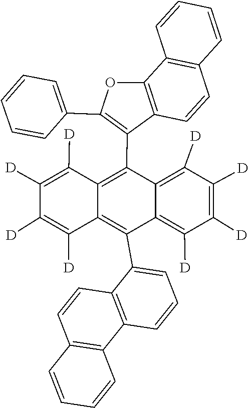 [SMILES: [2H]c1c([2H])c([2H])c2c(-c3c(-c4ccccc4)oc4c3ccc3ccccc34)c3c([2H])c([2H])c([2H])c([2H])c3c(-c3cccc4c3ccc3ccccc34)c2c1[2H]] | 65% | 605.78 |
| 2 | int 2-2 | B3-2 | 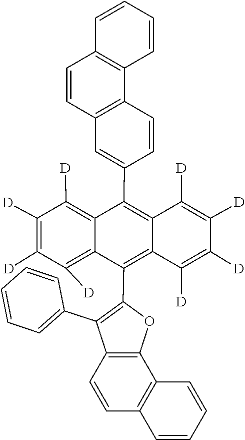 [SMILES: [2H]c1c([2H])c([2H])c2c(-c3oc4c(ccc5ccccc54)c3-c3ccccc3)c3c([2H])c([2H])c([2H])c([2H])c3c(-c3ccc4c(ccc5ccccc54)c3)c2c1[2H]] | 68% | 605.78 |
| 3 | int 2-3 | B3-3 | 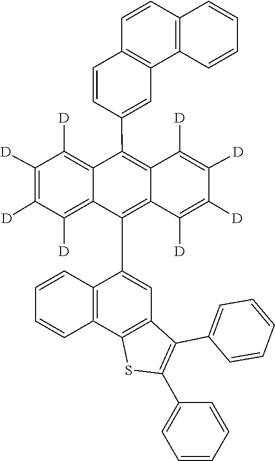 [SMILES: [2H]c1c([2H])c([2H])c2c(-c3cc4c(-c5ccccc5)c(-c5ccccc5)sc4c4ccccc34)c3c([2H])c([2H])c([2H])c([2H])c3c(-c3ccc4ccc5ccccc5c4c3)c2c1[2H]] | 60% | 697.94 |
| 4 | int 2-4 | B3-4 | 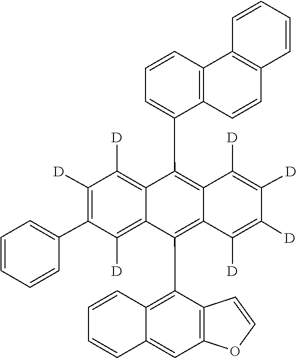 [SMILES: [2H]c1c([2H])c([2H])c2c(-c3cccc4c3ccc3ccccc34)c3c([2H])c([2H])c(-c4ccccc4)c([2H])c3c(-c3c4ccccc4cc4occc34)c2c1[2H]] | 67% | 604.77 |
| 5 | int 2-5 | B3-5 | 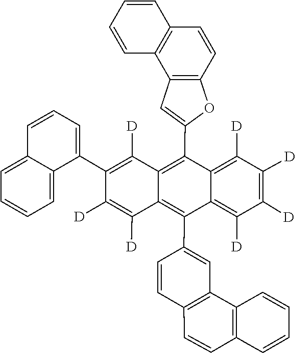 | 63% | 654.83 |
| 6 | int 2-6 | B3-6 | 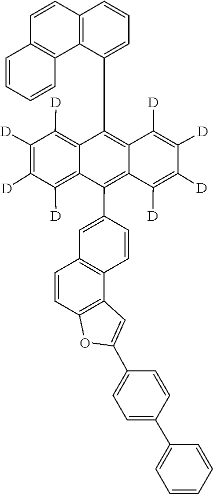 [SMILES: [2H]c1c([2H])c([2H])c2c(-c3cccc4ccc5ccccc5c34)c3c([2H])c([2H])c([2H])c([2H])c3c(-c3ccc4c(ccc5oc(-c6ccc(-c7ccccc7)cc6)cc54)c3)c2c1[2H]] | 60% | 681.88 |
| 7 | int 2-3 | B3-7 | 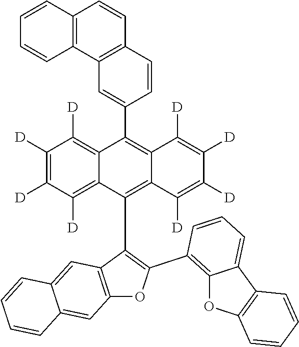 [SMILES: [2H]c1c([2H])c([2H])c2c(-c3c(-c4cccc5c4oc4ccccc45)oc4cc5ccccc5cc34)c3c([2H])c([2H])c([2H])c([2H])c3c(-c3ccc4ccc5ccccc5c4c3)c2c1[2H]] | 64% | 695.86 |
| 8 | int 2-1 | B3-8 | 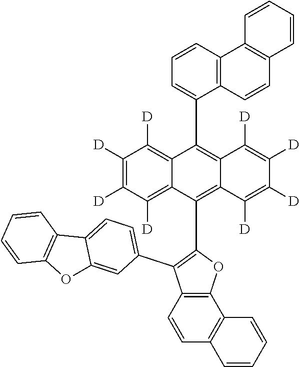 [SMILES: [2H]c1c([2H])c([2H])c2c(-c3cccc4c3ccc3ccccc34)c3c([2H])c([2H])c([2H])c([2H])c3c(-c3oc4c(ccc5ccccc54)c3-c3ccc4c(c3)oc3ccccc34)c2c1[2H]] | 61% | 695.86 |
| 9 | int 2-2 | B3-9 | 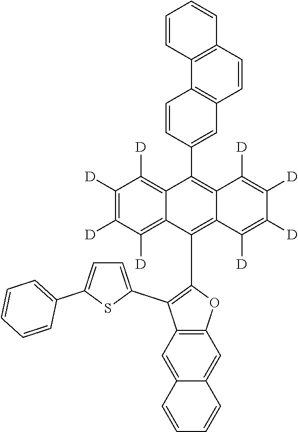 [SMILES: [2H]c1c([2H])c([2H])c2c(-c3oc4cc5ccccc5cc4c3-c3ccc(-c4ccccc4)s3)c3c([2H])c([2H])c([2H])c([2H])c3c(-c3ccc4c(ccc5ccccc54)c3)c2c1[2H]] | 63% | 687.90 |
| 10 | int 2-2 | B3-10 | 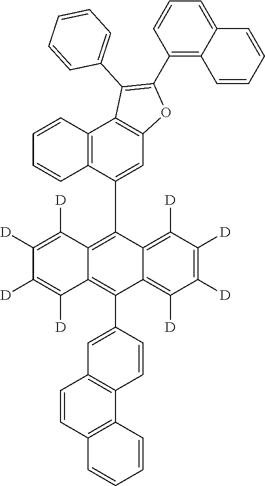 [SMILES: [2H]c1c([2H])c([2H])c2c(-c3cc4oc(-c5cccc6ccccc56)c(-c5ccccc5)c4c4ccccc34)c3c([2H])c([2H])c([2H])c([2H])c3c(-c3ccc4c(ccc5ccccc54)c3)c2c1[2H]] | 62% | 731.94 |
| 11 | int 2-1 | B3-11 | 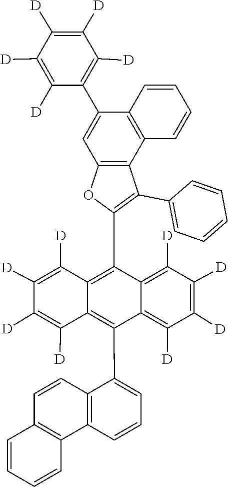 [SMILES: [2H]c1c([2H])c([2H])c(-c2cc3oc(-c4c5c([2H])c([2H])c([2H])c([2H])c5c(-c5cccc6c5ccc5ccccc56)c5c([2H])c([2H])c([2H])c([2H])c45)c(-c4ccccc4)c3c3ccccc23)c([2H])c1[2H]] | 60% | 686.91 |
| 12 | int 2-3 | B3-12 | 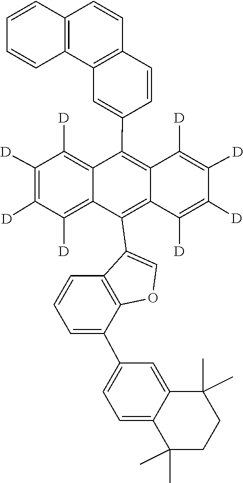 [SMILES: [2H]c1c([2H])c([2H])c2c(-c3coc4c(-c5ccc6c(c5)C(C)(C)CCC6(C)C)cccc34)c3c([2H])c([2H])c([2H])c([2H])c3c(-c3ccc4ccc5ccccc5c4c3)c2c1[2H]] | 68% | 665.92 |
| 13 | int 2-1 | B3-13 | 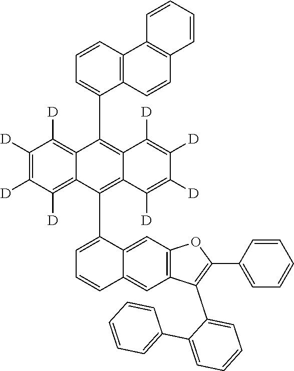 [SMILES: [2H]c1c([2H])c([2H])c2c(-c3cccc4c3ccc3ccccc34)c3c([2H])c([2H])c([2H])c([2H])c3c(-c3cccc4cc5c(-c6ccccc6-c6ccccc6)c(-c6ccccc6)oc5cc34)c2c1[2H]] | 63% | 757.97 |
| 14 | int 2-7 | B3-14 | 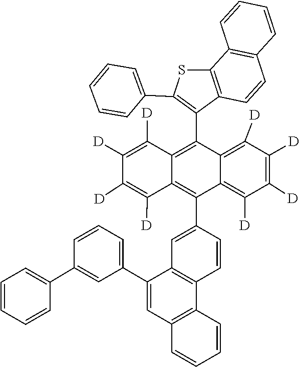 [SMILES: [2H]c1c([2H])c([2H])c2c(-c3c(-c4ccccc4)sc4c3ccc3ccccc34)c3c([2H])c([2H])c([2H])c([2H])c3c(-c3ccc4c(c3)c(-c3cccc(-c5ccccc5)c3)cc3ccccc34)c2c1[2H]] | 64% | 774.03 |
| 15 | int 2-8 | B3-15 | 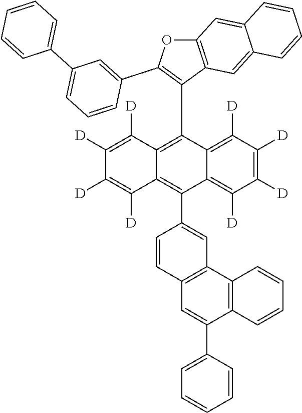 [SMILES: [2H]c1c([2H])c([2H])c2c(-c3c(-c4cccc(-c5ccccc5)c4)oc4cc5ccccc5cc34)c3c([2H])c([2H])c([2H])c([2H])c3c(-c3ccc4cc(-c5ccccc5)c5ccccc5c4c3)c2c1[2H]] | 65 % | 757.97 |
| 16 | int 2-9 | B3-16 | 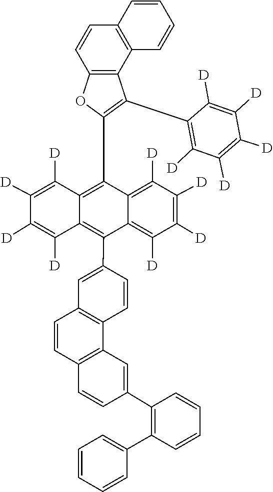 [SMILES: [2H]c1c([2H])c([2H])c(-c2c(-c3c4c([2H])c([2H])c([2H])c([2H])c4c(-c4ccc5c(ccc6ccc(-c7ccccc7-c7ccccc7)cc65)c4)c4c([2H])c([2H])c([2H])c([2H])c34)oc3ccc4ccccc4c23)c([2H])c1[2H]] | 67% | 763.00 |
| 17 | int 2-10 | B3-17 | 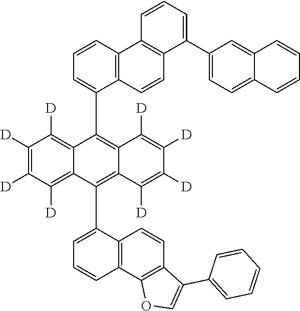 [SMILES: [2H]c1c([2H])c([2H])c2c(-c3cccc4c3ccc3c(-c5ccccc5)coc34)c3c([2H])c([2H])c([2H])c([2H])c3c(-c3cccc4c3ccc3c(-c5ccc6ccccc6c5)cccc34)c2c1[2H]] | 69% | 731.94 |
| 18 | int 2-3 | B3-18 | 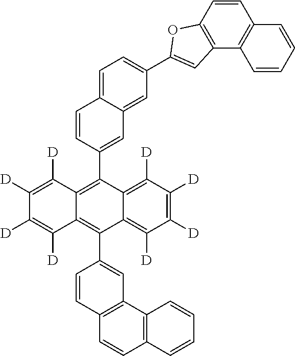 [SMILES: [2H]c1c([2H])c([2H])c2c(-c3ccc4ccc5ccccc5c4c3)c3c([2H])c([2H])c([2H])c([2H])c3c(-c3ccc4ccc(-c5cc6c(ccc7ccccc76)o5)cc4c3)c2c1[2H]] | 60% | 655.84 |
| 19 | int 2-11 | B3-19 | 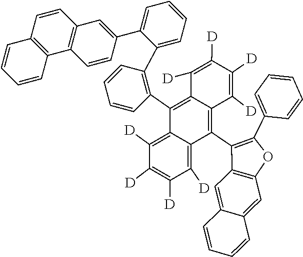 [SMILES: [2H]c1c([2H])c([2H])c2c(-c3c(-c4ccccc4)oc4cc5ccccc5cc34)c3c([2H])c([2H])c([2H])c([2H])c3c(-c3ccccc3-c3ccccc3-c3ccc4c(ccc5ccccc54)c3)c2c1[2H]] | 63% | 757.97 |
| 20 | int 2-12 | B3-20 | 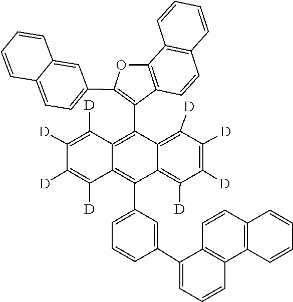 [SMILES: [2H]c1c([2H])c([2H])c2c(-c3c(-c4ccc5ccccc5c4)oc4c3ccc3ccccc34)c3c([2H])c([2H])c([2H])c([2H])c3c(-c3cccc(-c4cccc5c4ccc4ccccc45)c3)c2c1[2H]] | 61% | 731.94 |
| 21 | int 2-13 | B3-21 | 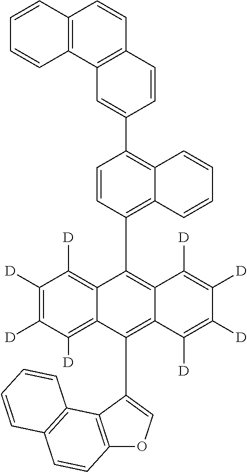 [SMILES: [2H]c1c([2H])c([2H])c2c(-c3coc4ccc5ccccc5c34)c3c([2H])c([2H])c([2H])c([2H])c3c(-c3ccc(-c4ccc5ccc6ccccc6c5c4)c4ccccc34)c2c1[2H]] | 65% | 655.84 |
| 22 | int 2-14 | B3-22 | 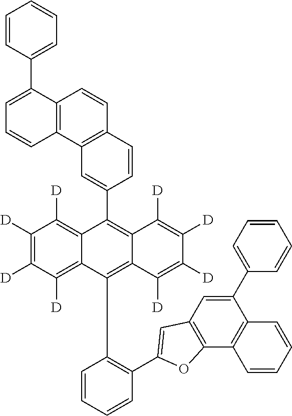 [SMILES: [2H]c1c([2H])c([2H])c2c(-c3ccccc3-c3cc4cc(-c5ccccc5)c5ccccc5c4o3)c3c([2H])c([2H])c([2H])c([2H])c3c(-c3ccc4ccc5c(-c6ccccc6)cccc5c4c3)c2c1[2H]] | 67% | 757.97 |
| 23 | int 2-2 | B3-23 | 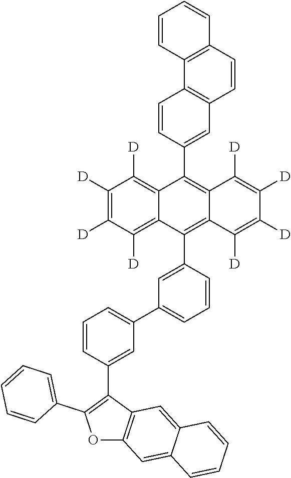 [SMILES: [2H]c1c([2H])c([2H])c2c(-c3ccc4c(ccc5ccccc54)c3)c3c([2H])c([2H])c([2H])c([2H])c3c(-c3cccc(-c4cccc(-c5c(-c6ccccc6)oc6cc7ccccc7cc56)c4)c3)c2c1[2H]] | 63% | 757.97 |
| 24 | int 2-1 | B3-24 | 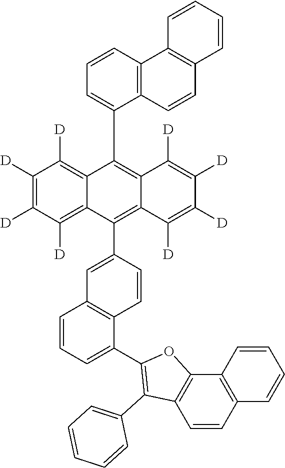 [SMILES: [2H]c1c([2H])c([2H])c2c(-c3cccc4c3ccc3ccccc34)c3c([2H])c([2H])c([2H])c([2H])c3c(-c3ccc4c(-c5oc6c(ccc7ccccc76)c5-c5ccccc5)cccc4c3)c2c1[2H]] | 66% | 731.94 |
| 25 | int 2-3 | B3-25 | 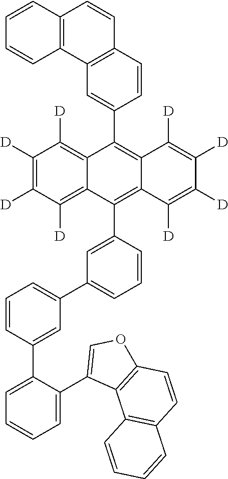 [SMILES: [2H]c1c([2H])c([2H])c2c(-c3ccc4ccc5ccccc5c4c3)c3c([2H])c([2H])c([2H])c([2H])c3c(-c3cccc(-c4cccc(-c5ccccc5-c5coc6ccc7ccccc7c56)c4)c3)c2c1[2H]] | 64% | 757.97 |
| 26 | int 2-1 | B3-26 | 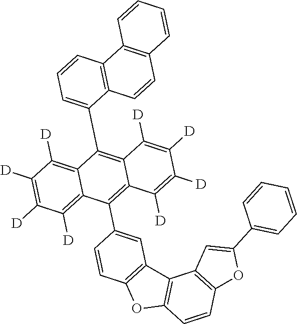 [SMILES: [2H]c1c([2H])c([2H])c2c(-c3cccc4c3ccc3ccccc34)c3c([2H])c([2H])c([2H])c([2H])c3c(-c3ccc4oc5ccc6oc(-c7ccccc7)cc6c5c4c3)c2c1[2H]] | 61% | 645.80 |
| 27 | int 2-2 | B3-27 | 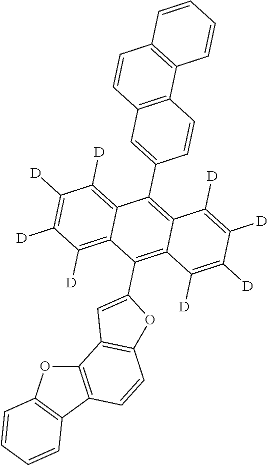 [SMILES: [2H]c1c([2H])c([2H])c2c(-c3cc4c(ccc5c6ccccc6oc45)o3)c3c([2H])c([2H])c([2H])c([2H])c3c(-c3ccc4c(ccc5ccccc54)c3)c2c1[2H]] | 62% | 569.70 |
| 28 | int 2-3 | B3-28 | 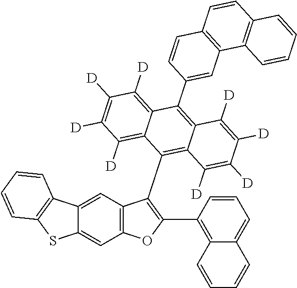 [SMILES: [2H]c1c([2H])c([2H])c2c(-c3c(-c4cccc5ccccc45)oc4cc5sc6ccccc6c5cc34)c3c([2H])c([2H])c([2H])c([2H])c3c(-c3ccc4ccc5ccccc5c4c3)c2c1[2H]] | 60% | 711.92 |
| 29 | int 2-6 | B3-29 | 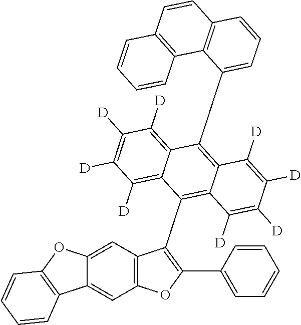 [SMILES: [2H]c1c([2H])c([2H])c2c(-c3cccc4ccc5ccccc5c34)c3c([2H])c([2H])c([2H])c([2H])c3c(-c3c(-c4ccccc4)oc4cc5c(cc34)oc3ccccc35)c2c1[2H]] | 63% | 645.80 |
| 30 | int 2-1 | B3-30 | 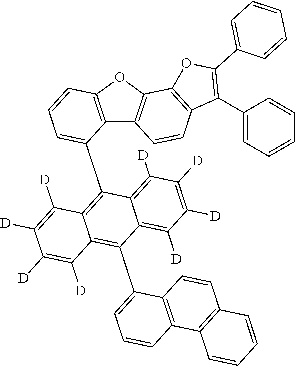 [SMILES: [2H]c1c([2H])c([2H])c2c(-c3cccc4oc5c(ccc6c(-c7ccccc7)c(-c7ccccc7)oc65)c34)c3c([2H])c([2H])c([2H])c([2H])c3c(-c3cccc4c3ccc3ccccc34)c2c1[2H]] | 64% | 721.90 |
| 31 | int 2-3 | B3-31 | 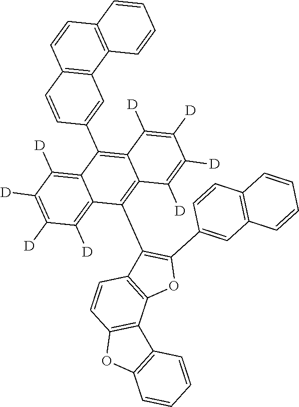 [SMILES: [2H]c1c([2H])c([2H])c2c(-c3c(-c4ccc5ccccc5c4)oc4c3ccc3oc5ccccc5c34)c3c([2H])c([2H])c([2H])c([2H])c3c(-c3ccc4ccc5ccccc5c4c3)c2c1[2H]] | 60% | 695.86 |

### 7) Synthesis of Chemical Formula 1D

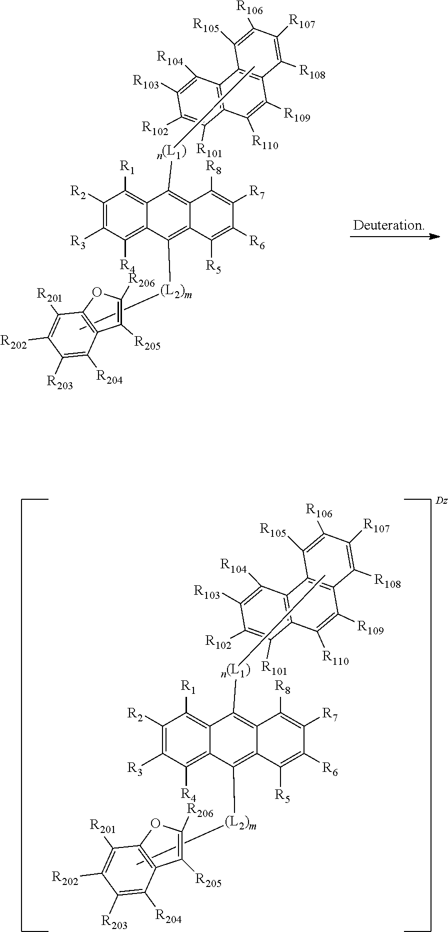

The reactant (1 eq) and trifluoromethanesulfonic acid (cat.) were put into C₆D₆ (10- to 50-fold mass ratio compared to the reactant), and the resulting mixture was stirred at 70° C. for 10 minutes to 100 minutes. After the reaction was completed, D₂O (excess) was added thereto, the resulting solution was stirred for 30 minutes, and then trimethylamine (excess) was added dropwise thereto. The reaction solution was transferred to a separatory funnel, and an extraction with water and chloroform was performed. The extract was dried over MgSO₄, and then recrystallized by heating with toluene to obtain the products (Compounds 32 to 35) of the following Table 9.

TABLE 9

| | | | | Product | | | | | Ratio | Ratio of | |
|---|---|---|---|---|---|---|---|---|---|---|---|
| | | | | theo- | | Product | Number | Number | of D | Total D | |
| | | | | retical | Reaction | actual | of total | of D | substi- | substi- | |
| Com- | | | Reactant | max | time | max | hydrogen | substi- | tutions | tutions | |
| pound | Reactant | Product | m/z | m/z | (min) | m/z | atoms | tutions | (%) | (%) | Yield |
| 32 | Com- pound 1 | 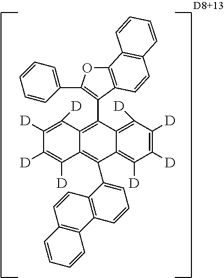 | 604 | 624 | 50 | 617 | 21 | 13 | 65% | 75% | 70% |
| 33 | Com- pound 3 | 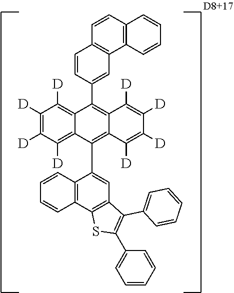 | 696 | 720 | 50 | 713 | 25 | 17 | 71% | 78% | 68% |
| 34 | Com- pound 29 | 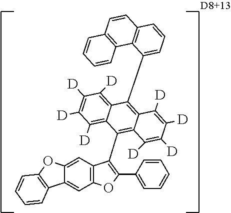 | 644 | 664 | 50 | 657 | 21 | 13 | 65% | 75% | 66% |
| 35 | Com- pound 31 | 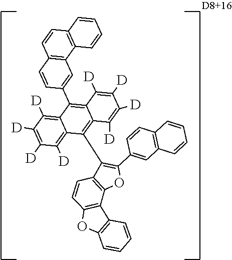 | 694 | 716 | 50 | 710 | 24 | 16 | 73% | 80% | 67% |

[Each product has a different degree of deuterium substitution depending on the reaction time, and the substitution rate was determined according to the maximum m/z (M+) value]

### <Example 1> Manufacture of OLED

As an anode, a substrate on which ITO/Ag/ITO were deposited to have a thickness of 70 Å/1,000 Å/70 Å was cut into a size of 50 mm×50 mm×0.5 mm, put into distilled water in which a dispersant was dissolved, and washed with ultrasonic waves. A product manufactured by Fischer Co., was used as the detergent, and distilled water twice filtered using a filter manufactured by Millipore Co., was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

HI-1 was thermally vacuum-deposited to have a thickness of 50 Å on the anode thus prepared, thereby forming a hole injection layer, and HT1 as a material which transports holes was vacuum-deposited to have a thickness of 1,150 Å thereon, thereby forming a hole transport layer. Next, a hole adjusting layer was formed using EB1 (150 Å). Next, the host compound 1 synthesized in Preparation Example 1 and a dopant BD1 (2 wt %) were vacuum-deposited to have a thickness of 360 Å, thereby forming a light emitting layer. Thereafter, HB1 was deposited to have a thickness of 50 Å, thereby forming an electron adjusting layer, and Compounds ET1 and Liq were mixed at 5:5 (mass ratio), thereby forming an electron transport layer having a thickness of 250 Å. Sequentially, magnesium and lithium fluoride (LiF) were deposited to have a thickness of 50 Å to form a film as an electron injection layer <EIL>, magnesium and silver (1:4) were used to form a cathode having a thickness of 200 Å, and then CP1 was deposited to have a thickness of 600 Å, thereby completing a device. In the aforementioned procedure, the deposition rates of the organic materials were each maintained at 1 Å/sec.

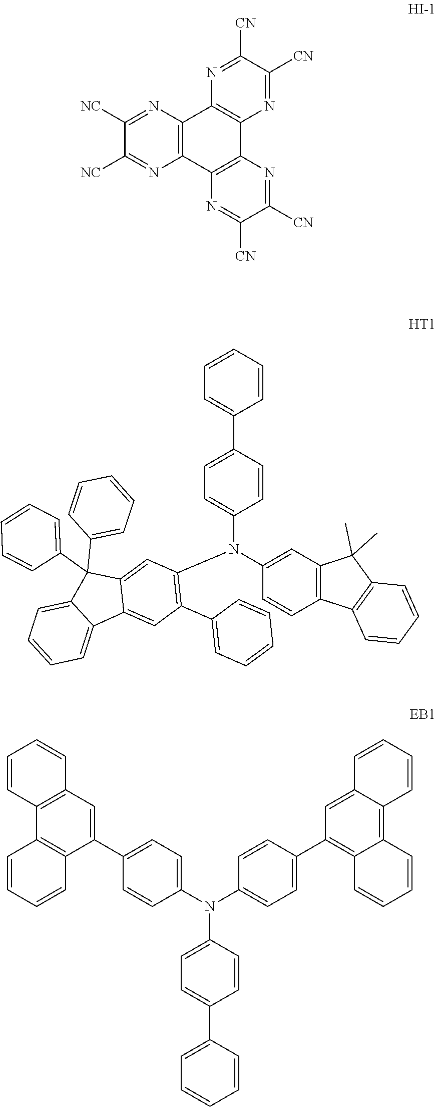 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3cc4c(cc3-c3ccccc3)-c3ccccc3C4(c3ccccc3)c3ccccc3)cc21 ; c1ccc(-c2ccc(N(c3ccc(-c4cc5ccccc5c5ccccc45)cc3)c3ccc(-c4cc5ccccc5c5ccccc45)cc3)cc2)cc1]

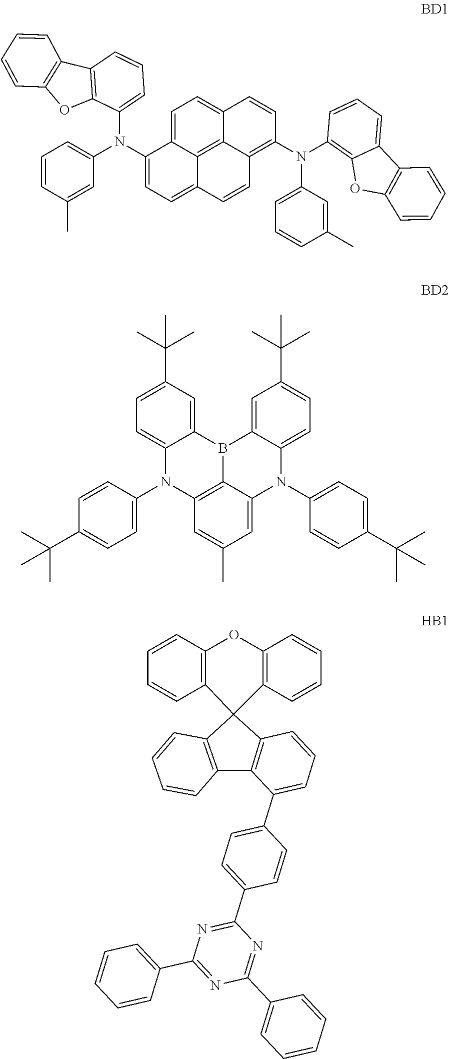 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2nc(-c3ccccc3)nc(-c3ccc(-c4cccc5c4-c4ccccc4C54c5ccccc5Oc5ccccc54)cc3)n2)cc1]

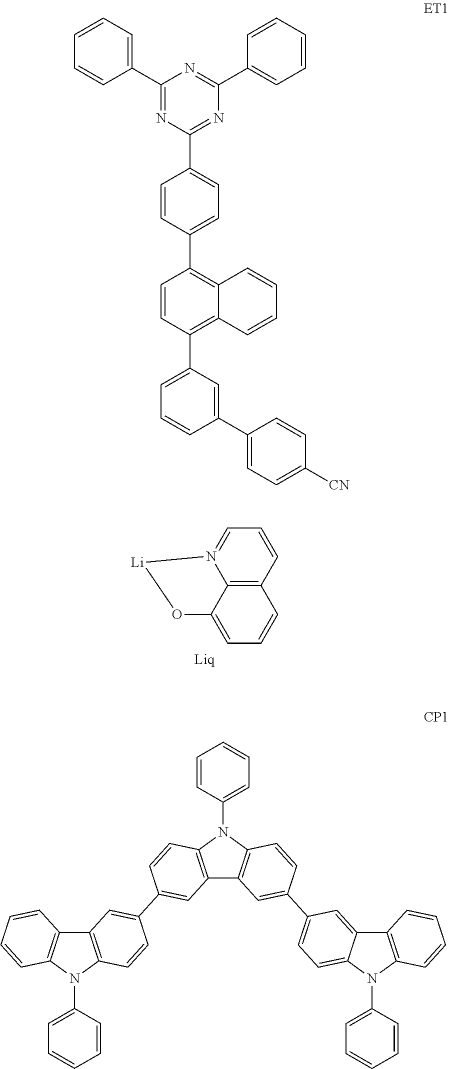

### Comparative Examples 1 to 8 and Examples 2 to 39

Devices were manufactured in the same manner as in Example 1, except that in Comparative Examples 1 to 8 and Examples 2 to 39, the materials shown in the following [Table 10] were used as materials for the light emitting layer.

For the devices manufactured in Comparative Examples 1 to 8 and Examples 1 to 39, the driving voltage, light emitting efficiency, and a time (LT95) for reaching a 95% value compared to the initial luminance were measured at a current density of 20 mA/cm². The results are shown in the following [Table 10].

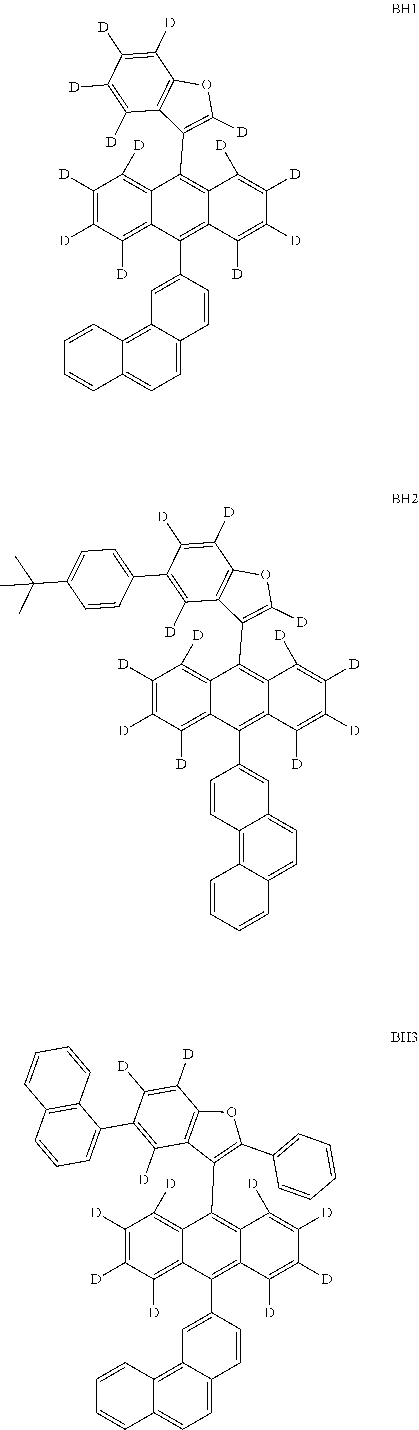 [SMILES: [2H]c1oc2c([2H])c([2H])c([2H])c([2H])c2c1-c1c2c([2H])c([2H])c([2H])c([2H])c2c(-c2ccc3ccc4ccccc4c3c2)c2c([2H])c([2H])c([2H])c([2H])c12 ; [2H]c1oc2c([2H])c([2H])c(-c3ccc(C(C)(C)C)cc3)c([2H])c2c1-c1c2c([2H])c([2H])c([2H])c([2H])c2c(-c2ccc3c(ccc4ccccc43)c2)c2c([2H])c([2H])c([2H])c([2H])c12 ; [2H]c1c(-c2cccc3ccccc23)c([2H])c2c(-c3c4c([2H])c([2H])c([2H])c([2H])c4c(-c4ccc5ccc6ccccc6c5c4)c4c([2H])c([2H])c([2H])c([2H])c34)c(-c3ccccc3)oc2c1[2H]]

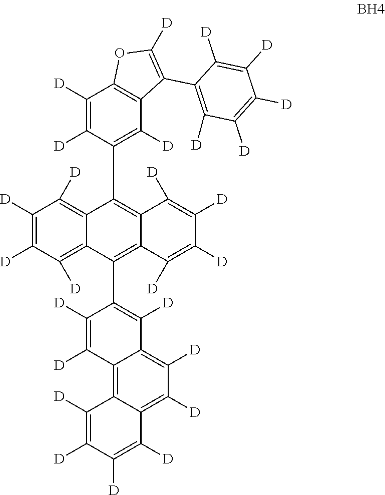 [SMILES: [2H]c1oc2c([2H])c([2H])c(-c3c4c([2H])c([2H])c([2H])c([2H])c4c(-c4c([2H])c([2H])c5c(c4[2H])c([2H])c([2H])c4c([2H])c([2H])c([2H])c([2H])c45)c4c([2H])c([2H])c([2H])c([2H])c34)c([2H])c2c1-c1c([2H])c([2H])c([2H])c([2H])c1[2H]]

TABLE 10

| | | | Device results | | | |
|---|---|---|---|---|---|---|
| | | | | | Color | Service life |
| Experimental | Light emitting layer | | Voltage (V) | Cd/A | coordinate | (T95, h) |
| Example | Host | Dopant | (@20 mA/cm²) | (@20 mA/cm²) | (x, y) | (@20 mA/cm²) |
| Comparative | BH1 | BD1 | 3.71 | 5.46 | (0.140, | 53.2 |
| Example 1 | | | | | 0.045) | |
| Comparative | BH2 | BD1 | 3.75 | 5.32 | (0.140, | 51.6 |
| Example 2 | | | | | 0.047) | |
| Comparative | BH3 | BD1 | 3.73 | 5.43 | (0.141, | 54.0 |
| Example 3 | | | | | 0.047) | |
| Comparative | BH4 | BD1 | 3.71 | 5.45 | (0.142, | 58.0 |
| Example 4 | | | | | 0.047) | |
| Comparative | BH1 | BD2 | 3.80 | 5.53 | (0.132, | 48.1 |
| Example 5 | | | | | 0.044) | |
| Comparative | BH2 | BD2 | 3.83 | 5.40 | (0.133, | 46.5 |
| Example 6 | | | | | 0.045) | |
| Comparative | BH3 | BD2 | 3.81 | 5.51 | (0.132, | 51.4 |
| Example 7 | | | | | 0.045) | |
| Comparative | BH4 | BD2 | 3.82 | 5.53 | (0.133, | 50.1 |
| Example 8 | | | | | 0.045) | |
| Example 1 | Compound | BD1 | 3.54 | 5.89 | (0.140, | 64.8 |
| | 1 | | | | 0.051) | |
| Example 2 | Compound | BD1 | 3.53 | 5.80 | (0.140, | 65.3 |
| | 2 | | | | 0.052) | |
| Example 3 | Compound | BD1 | 3.54 | 5.83 | (0.140, | 60.8 |
| | 3 | | | | 0.051) | |
| Example 4 | Compound | BD1 | 3.55 | 5.75 | (0.140, | 63.2 |
| | 4 | | | | 0.051) | |
| Example 5 | Compound | BD1 | 3.58 | 5.76 | (0.143, | 61.9 |
| | 5 | | | | 0.063) | |
| Example 6 | Compound | BD1 | 3.54 | 5.78 | (0.140, | 63.0 |
| | 6 | | | | 0.053) | |
| Example 7 | Compound | BD1 | 3.53 | 5.80 | (0.140, | 64.2 |
| | 7 | | | | 0.054) | |
| Example 8 | Compound | BD1 | 3.56 | 5.81 | (0.140, | 64.0 |
| | 8 | | | | 0.054) | |
| Example 9 | Compound | BD1 | 3.57 | 5.74 | (0.141, | 62.8 |
| | 9 | | | | 0.053) | |
| Example 10 | Compound | BD1 | 3.51 | 5.79 | (0.141, | 63.6 |
| | 10 | | | | 0.053) | |
| Example 11 | Compound | BD1 | 3.56 | 5.77 | (0.141, | 66.1 |
| | 11 | | | | 0.053) | |
| Example 12 | Compound | BD1 | 3.57 | 5.78 | (0.140, | 64.5 |
| | 12 | | | | 0.052) | |
| Example 13 | Compound | BD1 | 3.55 | 5.80 | (0.140, | 65.0 |
| | 13 | | | | 0.054) | |
| Example 14 | Compound | BD1 | 3.57 | 5.71 | (0.141, | 63.0 |
| | 14 | | | | 0.053) | |
| Example 15 | Compound | BD1 | 3.51 | 5.68 | (0.140, | 64.6 |
| | 15 | | | | 0.054) | |
| Example 16 | Compound | BD1 | 3.51 | 5.76 | (0.140, | 63.1 |
| | 16 | | | | 0.054) | |
| Example 17 | Compound | BD1 | 3.50 | 5.69 | (0.141, | 66.5 |
| | 17 | | | | 0.053) | |
| Example 18 | Compound | BD1 | 3.59 | 5.80 | (0.140, | 64.6 |
| | 18 | | | | 0.054) | |
| Example 19 | Compound | BD1 | 3.56 | 5.88 | (0.140, | 67.0 |
| | 19 | | | | 0.055) | |
| Example 20 | Compound | BD1 | 3.60 | 5.83 | (0.140, | 68.4 |
| | 20 | | | | 0.055) | |
| Example 21 | Compound | BD1 | 3.57 | 5.73 | (0.140, | 62.1 |
| | 21 | | | | 0.055) | |
| Example 22 | Compound | BD1 | 3.55 | 5.77 | (0.140, | 68.4 |
| | 22 | | | | 0.054) | |
| Example 23 | Compound | BD1 | 3.58 | 5.68 | (0.140, | 66.7 |
| | 23 | | | | 0.055) | |
| Example 24 | Compound | BD1 | 3.51 | 5.77 | (0.140, | 68.4 |
| | 24 | | | | 0.054) | |
| Example 25 | Compound | BD1 | 3.54 | 5.73 | (0.140, | 63.1 |
| | 25 | | | | 0.054) | |
| Example 26 | Compound | BD1 | 3.51 | 5.65 | (0.141, | 62.8 |
| | 26 | | | | 0.055) | |
| Example 27 | Compound | BD1 | 3.48 | 5.68 | (0.141, | 63.0 |
| | 27 | | | | 0.054) | |
| Example 28 | Compound | BD1 | 3.49 | 5.62 | (0.140, | 61.9 |
| | 28 | | | | 0.053) | |
| Example 29 | Compound | BD1 | 3.50 | 5.68 | (0.140, | 63.2 |
| | 29 | | | | 0.053) | |
| Example 30 | Compound | BD1 | 3.49 | 5.70 | (0.141, | 64.0 |
| | 30 | | | | 0.055) | |
| Example 31 | Compound | BD1 | 3.47 | 5.69 | (0.141, | 62.9 |
| | 31 | | | | 0.054) | |
| Example 32 | Compound | BD1 | 3.55 | 5.89 | (0.140, | 75.3 |
| | 32 | | | | 0.051) | |
| Example 33 | Compound | BD1 | 3.55 | 5.83 | (0.140, | 68.4 |
| | 33 | | | | 0.051) | |
| Example 34 | Compound | BD1 | 3.50 | 5.67 | (0.140, | 71.3 |
| | 34 | | | | 0.053) | |
| Example 35 | Compound | BD1 | 3.47 | 5.69 | (0.141, | 69.5 |
| | 35 | | | | 0.054) | |
| Example 36 | Compound | BD2 | 3.63 | 5.95 | (0.132, | 70.1 |
| | 32 | | | | 0.045) | |
| Example 37 | Compound | BD2 | 3.65 | 5.91 | (0.132, | 63.1 |
| | 33 | | | | 0.045) | |
| Example 38 | Compound | BD2 | 3.58 | 5.78 | (0.131, | 65.4 |
| | 34 | | | | 0.047) | |
| Example 39 | Compound | BD2 | 3.54 | 5.82 | (0.132, | 63.5 |
| | 35 | | | | 0.046) | |

The results in Table 10 above are for the case of having a device structure in which a light emitting layer is formed, in which the host of the light emitting layer is the sole host, and in which the compound in the claims of the present application is composed of the host of the light emitting layer.

In Comparative Example 1, the 3rd carbon of a phenanthrene is bonded to No. 10 position of an anthracene, the 2nd carbon of an unsubstituted benzofuran is bonded to No. 9 position of the anthracene, and deuterium is introduced into the anthracene and the unsubstituted benzofuran, and the present invention is different from Comparative Example 1, in that when the 2nd carbon of the benzofuran is bonded to No. 9 position of the anthracene, the present invention has one additional substituent. The compounds of the Examples of the present application have structural stabilization and carrier injection and migration speeds different from those of the Comparative Examples as a benzofuran core condensed with benzene or benzofuran is introduced, so that it can be confirmed that the present device structure shows excellent performance, particularly in terms of voltage and service life.

Further, Examples 14 to 17 show excellent device characteristics compared to Comparative Examples 1 and 2 by additionally introducing a substituent into the phenanthrene core under the aforementioned conditions.

In Examples 22 to 25, it can be seen that the electrical characteristics may be adjusted by introducing a linker, and the service life may be improved while maintaining the advantages of the corresponding structure.

Examples 26 to 31 show characteristics having further advantages in terms of voltage because a benzofuran core condensed with benzofuran is introduced.

In addition, Examples 32 to 35 show that additional service life improvement effect can be achieved despite the cost disadvantage while showing an additional service life increase of 10 to 15% on average through additional deuterium substitution, and Examples 36 to 39 can show the device consistency when various dopants are introduced because changing the dopant (pyrene dopant→boron dopant) maintains the tendency of the device while maintaining the change of the dopant characteristics.

### <Example 40> Manufacture of OLED

As an anode, a substrate on which ITO/Ag/ITO were deposited to have a thickness of 70 Å/1,000 Å/70 Å was cut into a size of 50 mm×50 mm×0.5 mm, put into distilled water in which a dispersant was dissolved, and washed with ultrasonic waves. A product manufactured by Fischer Co., was used as the detergent, and distilled water twice filtered using a filter manufactured by Millipore Co., was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

HI-1 was thermally vacuum-deposited to have a thickness of 50 Å on the anode thus prepared, thereby forming a hole injection layer, and HT1 as a material which transports holes was vacuum-deposited to have a thickness of 1,150 Å thereon, thereby forming a hole transport layer. Next, a hole adjusting layer was formed using EB1 (150 Å). Next, as a light emitting layer, the host compound 2 synthesized in Preparation Example 1, BH13, and a dopant BD1 (2 wt %) were vacuum-co-deposited to have a thickness of 360 Å, thereby forming a light emitting layer. Thereafter, HB1 was deposited to have a thickness of 50 Å, thereby forming an electron adjusting layer, and Compounds ET1 and Liq were mixed at 5:5 (mass ratio), thereby forming an electron transport layer having a thickness of 250 Å. Sequentially, magnesium and lithium fluoride (LiF) were deposited to have a thickness of 50 Å to form a film as an electron injection layer <EIL>, magnesium and silver (1:4) were used to form a cathode having a thickness of 200 Å, and then CP1 was deposited to have a thickness of 600 Å, thereby completing a device. In the aforementioned procedure, the deposition rates of the organic materials were each maintained at 1 Å/sec.

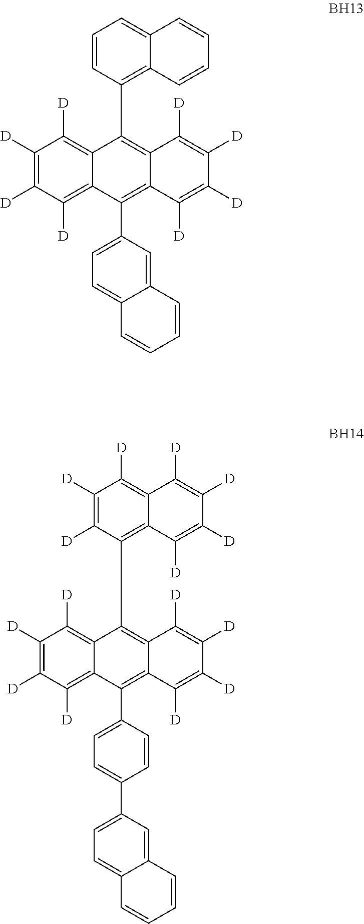 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3c4c([2H])c([2H])c([2H])c([2H])c4c(-c4ccc(-c5ccc6ccccc6c5)cc4)c4c([2H])c([2H])c([2H])c([2H])c34)c([2H])c([2H])c([2H])c2c1[2H]]

### Comparative Examples 9 to 14 and Examples 41 to 44

Devices were manufactured in the same manner as in Example 27, except that in Comparative Examples 9 to 14 and Examples 41 to 44, the materials shown in the following Table 2 were used as materials for the light emitting layer.

However, the host of the light emitting layer is an embodiment in which two hosts as shown in Table 11 were co-deposited using other evaporation sources during device manufacture to form a light emitting layer.

For the devices manufactured in Comparative Examples 9 to 14 and Examples 40 to 44, the driving voltage, light emitting efficiency, and a time (LT95) for reaching a 95% value compared to the initial luminance were measured at a current density of 20 mA/cm². The results are shown in the following [Table 11].

TABLE 11

| | Light emitting layer | | Device results | | | |
|---|---|---|---|---|---|---|
| | First host:Second host | | | | Color | Service life |
| Experimental | (Ratio, mixing | | Voltage (V) | Cd/A | coordinate | (T95, h) |
| Example | method) | Dopant | (@20 mA/cm²) | (@20 mA/cm²) | (x, y) | (@20 mA/cm²) |
| Comparative | BH1:BH13 | BD1 | 3.65 | 5.58 | (0.139, | 57.2 |
| Example 9 | (1:1) | | | | 0.045) | |
| Comparative | BH2:BH14 | BD1 | 3.66 | 5.49 | (0.140, | 56.4 |
| Example 10 | (1:1) | | | | 0.046) | |
| Comparative | BH3:BH13 | BD1 | 3.62 | 5.52 | (0.141, | 61.3 |
| Example 11 | (1:1) | | | | 0.047) | |
| Comparative | BH4:BH14 | BD1 | 3.65 | 5.53 | (0.142, | 63.1 |
| Example 12 | (1:1) | | | | 0.046) | |
| Comparative | BH1:BH13 | BD2 | 3.73 | 5.68 | (0.132, | 51.9 |
| Example 13 | (1:1) | | | | 0.044) | |
| Comparative | BH4:BH14 | BD2 | 3.74 | 5.61 | (0.133, | 55.3 |
| Example 14 | (1:1) | | | | 0.045) | |
| Example 40 | Compound32:BH13 | BD1 | 3.59 | 6.01 | (0.140, | 80.1 |
| | (1:1) | | | | 0.049) | |
| Example 41 | Compound33:BH13 | BD1 | 3.54 | 5.97 | (0.140, | 75.9 |
| | (1:1) | | | | 0.049) | |
| Example 42 | Compound34:BH14 | BD1 | 3.55 | 5.89 | (0.140, | 78.3 |
| | (1:1) | | | | 0.050) | |
| Example 43 | Compound35:BH14 | BD1 | 3.51 | 5.84 | (0.141, | 76.1 |
| | (1:1) | | | | 0.051) | |
| Example 44 | Compound32:BH13 | BD2 | 3.67 | 6.15 | (0.132, | 73.5 |
| | (1:1) | | | | 0.043) | |

The results in [Table 11] above are for the case where the host in the light emitting layer is composed of a mixed host (a mixed host of two anthracene species).

Comparative Examples 9 to 12 show examples in which mixed hosts were formed using BH13 and BH14 among widely used aryl-based anthracene compounds, and BH1, BH2, BH3, and BH4 among the structures of the present application, as comparative examples, and it was observed that devices having light emitting layers formed using the corresponding combinations showed device results that were partially improved in all aspects of voltage, efficiency, and service life. Furthermore, Comparative Examples 13 and 14 show the same tendency even when boron-based blue dopants are applied.

In Examples 40 to 44, Compounds 28 to 31 among the example compounds of the claims of the present application were mixed with the previous BH13 or BH14 to form mixed hosts, and the device results were observed, and it can be seen that the compounds of the claims of the present application, which had excellent characteristics compared to Comparative Examples 9 to 14, have the same tendency even under mixed host conditions.

What is claimed is:

1. An organic compound of the following Chemical Formula 1: 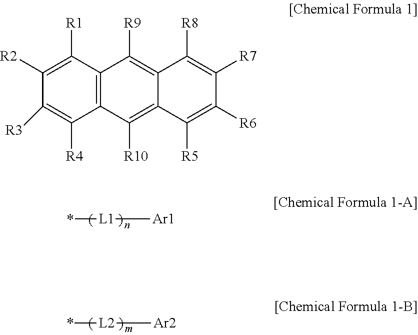
wherein, in Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C, 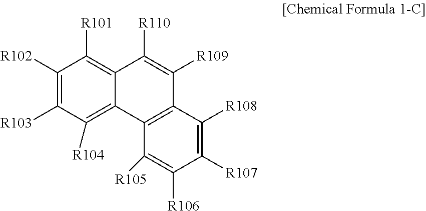
in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is necessarily bonded to L1,
Ar2 is the following Chemical Formula 1-D, 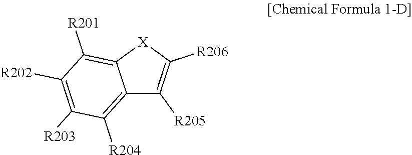
in Chemical Formula 1-D,
X is O or S,
R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups,
the substituted or unsubstituted ring group; or any one of R201 to R206 not forming the substituted or unsubstituted ring group is bonded to L2, and
* is a moiety bonded to Chemical Formula 1.

2. The organic compound of claim 1, wherein the substituted or unsubstituted ring group of Chemical Formula 1-D is any one of the following 1-D-1 to 1-D-8: 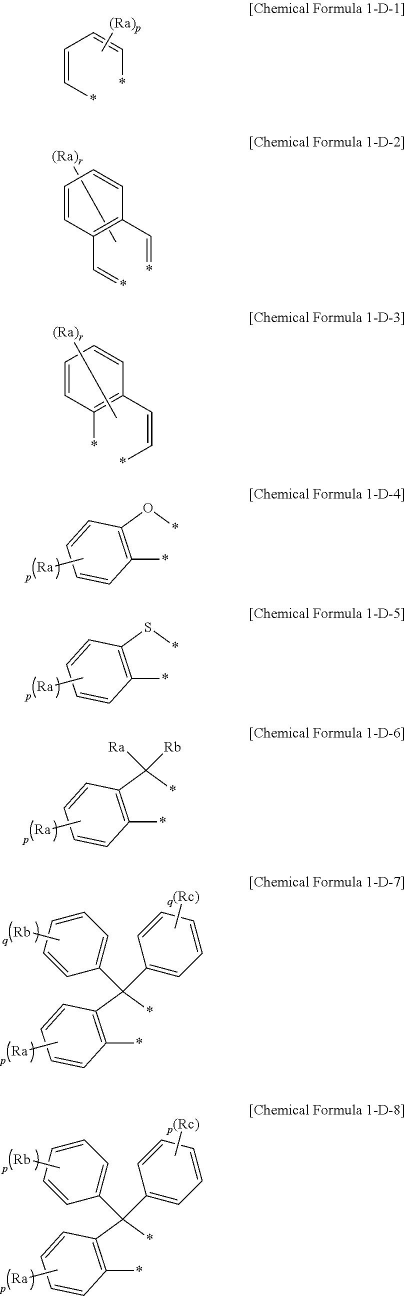
in Chemical Formulae 1-D-1 to 1-D-8,
Ra to Rc are the same as or different from each other, and are each independently hydrogen; deuterium; a substituted or unsubstituted alkyl group; a substituted or unsubstituted aryl group; or a substituted or unsubstituted heterocyclic group, or optionally bonded to an adjacent group to form a substituted or unsubstituted ring group, or bonded to L2 above,
p is an integer from 1 to 4, and when p is 2 or higher, two or more Ra's, Rb's, or Rc's are the same as or different from each other,
q is an integer from 1 to 5, and when q is 2 or higher, two or more Rb's or Rc's are the same as or different from each other,
r is an integer from 1 to 6, and when r is 2 or higher, two or more Ra's are the same as or different from each other, and
* is a moiety bonded to an adjacent pair of R201 to R206 in Chemical Formula 1-D.

3. The organic compound of claim 1, wherein Chemical Formula 1 is any one of the following Chemical Formulae 1-1 to 1-4: 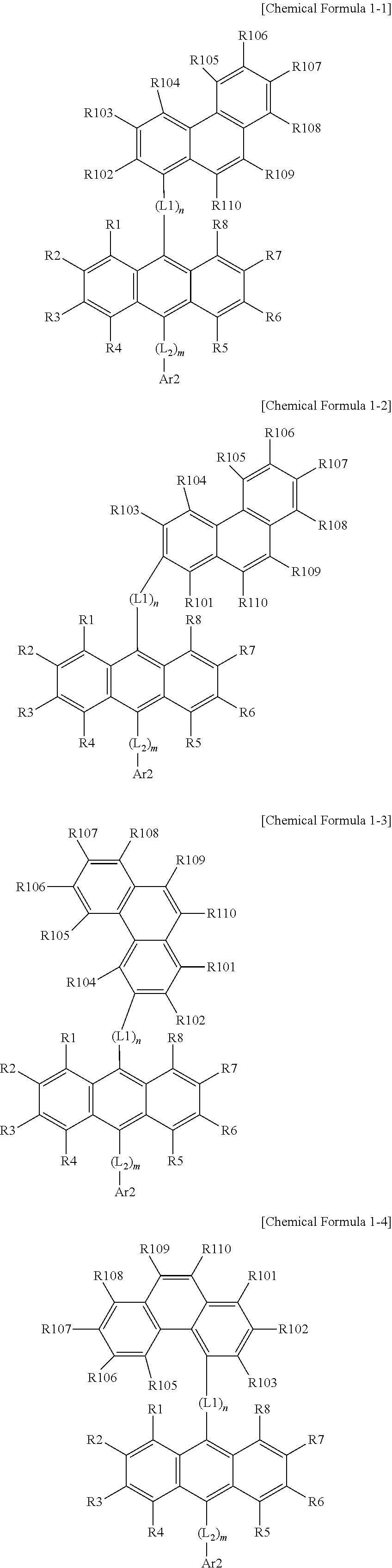
in Chemical Formulae 1-1 to 1-4,
the definitions of L1, L2, Ar2, n, and m are the same as the definitions in Chemical Formula 1 above,
R1 to R8 are each independently hydrogen; deuterium; a substituted or unsubstituted alkyl group; a substituted or unsubstituted cycloalkyl group; a substituted or unsubstituted aryl group; or a substituted or unsubstituted heteroaryl group, and
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group.

4. The organic compound of claim 1, wherein Chemical Formula 1 is any one of the following Chemical Formulae 1-5 to 1-10: 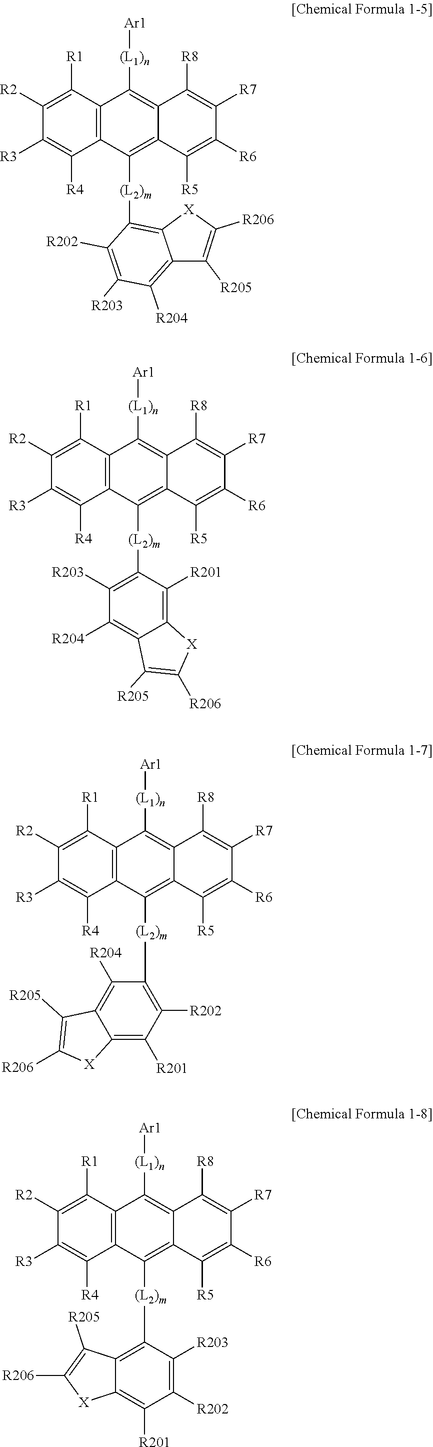 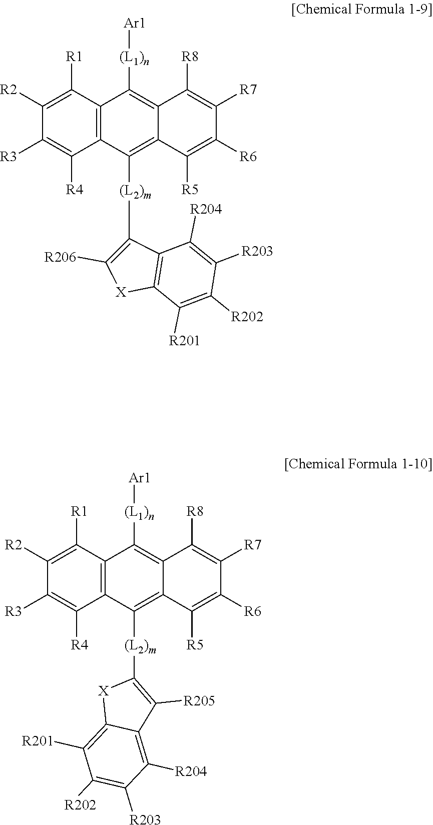
in Chemical Formulae 1-5 to 1-10,
the definitions of L1, L2, Ar1, X, n, and m are the same as the definitions in Chemical Formula 1 above,
R1 to R8 are each independently hydrogen; deuterium; a substituted or unsubstituted alkyl group; a substituted or unsubstituted cycloalkyl group; a substituted or unsubstituted aryl group; or a substituted or unsubstituted heteroaryl group, and
R201 to R206 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R206 is bonded to each other to necessarily form one or more substituted or unsubstituted ring groups.

5. The organic compound of claim 1, wherein R9 of Chemical Formula 1 above is bonded to L1 of Chemical Formula 1-A above, R10 of Chemical Formula 1 above is bonded to L2 of Chemical Formula 1-B above, and R1 to R8 of Chemical Formula 1 above are all deuterium.

6. The organic compound of claim 1, wherein a deuterium substitution rate of Chemical Formula 1 above is 15% or more.

7. The organic compound of claim 1, wherein Chemical Formula 1 is any one of the following compounds:

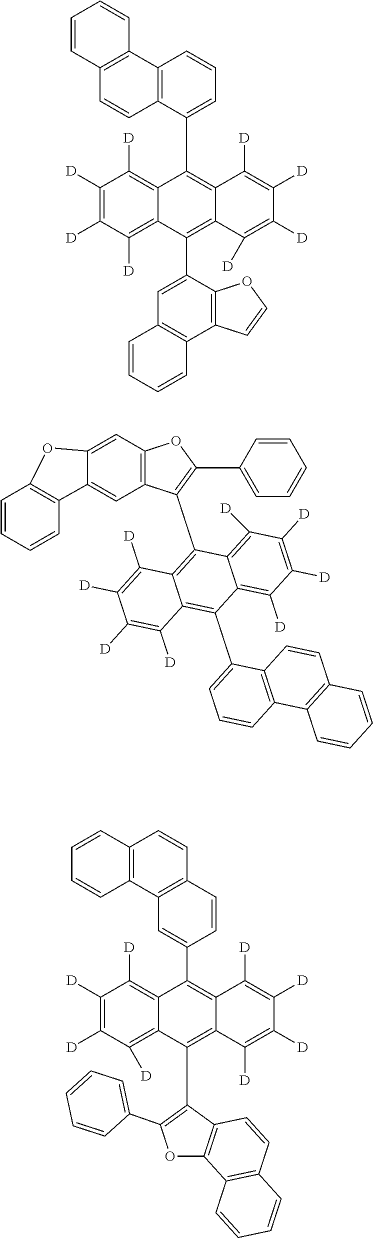 [SMILES: [2H]c1c([2H])c([2H])c2c(-c3cc4ccccc4c4ccoc34)c3c([2H])c([2H])c([2H])c([2H])c3c(-c3cccc4c3ccc3ccccc34)c2c1[2H] ; [2H]c1c([2H])c([2H])c2c(-c3cccc4c3ccc3ccccc34)c3c([2H])c([2H])c([2H])c([2H])c3c(-c3c(-c4ccccc4)oc4cc5oc6ccccc6c5cc34)c2c1[2H] ; [2H]c1c([2H])c([2H])c2c(-c3c(-c4ccccc4)oc4c3ccc3ccccc34)c3c([2H])c([2H])c([2H])c([2H])c3c(-c3ccc4ccc5ccccc5c4c3)c2c1[2H]]

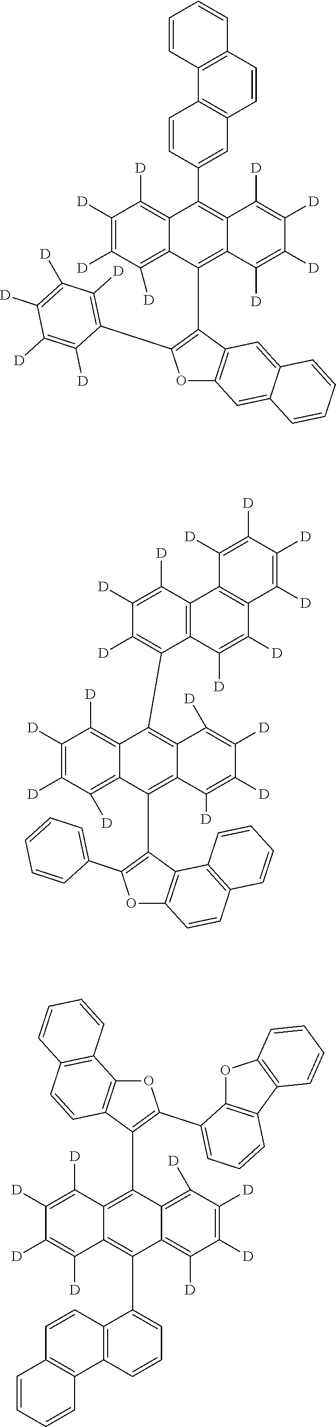 [SMILES: [2H]c1c([2H])c([2H])c(-c2oc3cc4ccccc4cc3c2-c2c3c([2H])c([2H])c([2H])c([2H])c3c(-c3ccc4c(ccc5ccccc54)c3)c3c([2H])c([2H])c([2H])c([2H])c23)c([2H])c1[2H] ; [2H]c1c([2H])c([2H])c2c(-c3c(-c4ccccc4)oc4ccc5ccccc5c34)c3c([2H])c([2H])c([2H])c([2H])c3c(-c3c([2H])c([2H])c([2H])c4c3c([2H])c([2H])c3c([2H])c([2H])c([2H])c([2H])c34)c2c1[2H] ; [2H]c1c([2H])c([2H])c2c(-c3c(-c4cccc5c4oc4ccccc45)oc4c3ccc3ccccc34)c3c([2H])c([2H])c([2H])c([2H])c3c(-c3cccc4c3ccc3ccccc34)c2c1[2H]]

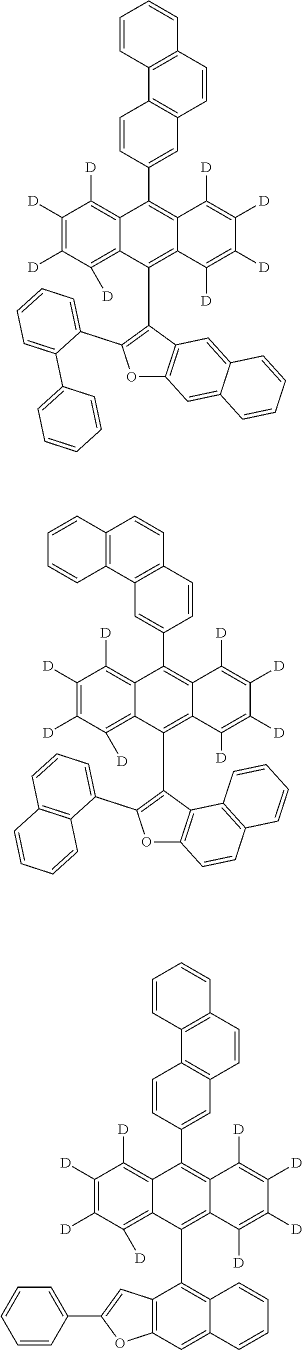 [SMILES: [2H]c1c([2H])c([2H])c2c(-c3c(-c4ccccc4-c4ccccc4)oc4cc5ccccc5cc34)c3c([2H])c([2H])c([2H])c([2H])c3c(-c3ccc4c(ccc5ccccc54)c3)c2c1[2H] ; [2H]c1c([2H])c([2H])c2c(-c3c(-c4cccc5ccccc45)oc4ccc5ccccc5c34)c3c([2H])c([2H])c([2H])c([2H])c3c(-c3ccc4ccc5ccccc5c4c3)c2c1[2H] ; [2H]c1c([2H])c([2H])c2c(-c3c4ccccc4cc4oc(-c5ccccc5)cc34)c3c([2H])c([2H])c([2H])c([2H])c3c(-c3ccc4c(ccc5ccccc54)c3)c2c1[2H]]

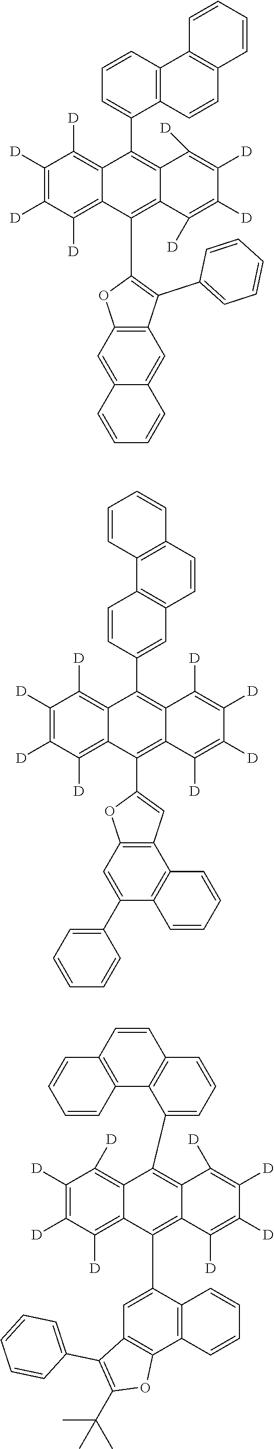

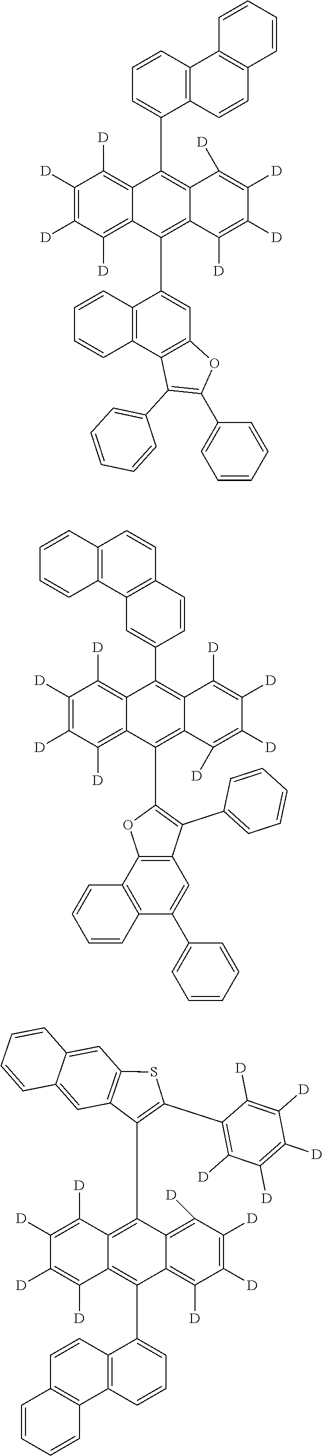 [SMILES: [2H]c1c([2H])c([2H])c2c(-c3cc4oc(-c5ccccc5)c(-c5ccccc5)c4c4ccccc34)c3c([2H])c([2H])c([2H])c([2H])c3c(-c3cccc4c3ccc3ccccc34)c2c1[2H] ; [2H]c1c([2H])c([2H])c2c(-c3oc4c(cc(-c5ccccc5)c5ccccc54)c3-c3ccccc3)c3c([2H])c([2H])c([2H])c([2H])c3c(-c3ccc4ccc5ccccc5c4c3)c2c1[2H] ; [2H]c1c([2H])c([2H])c(-c2sc3cc4ccccc4cc3c2-c2c3c([2H])c([2H])c([2H])c([2H])c3c(-c3cccc4c3ccc3ccccc34)c3c([2H])c([2H])c([2H])c([2H])c23)c([2H])c1[2H]]

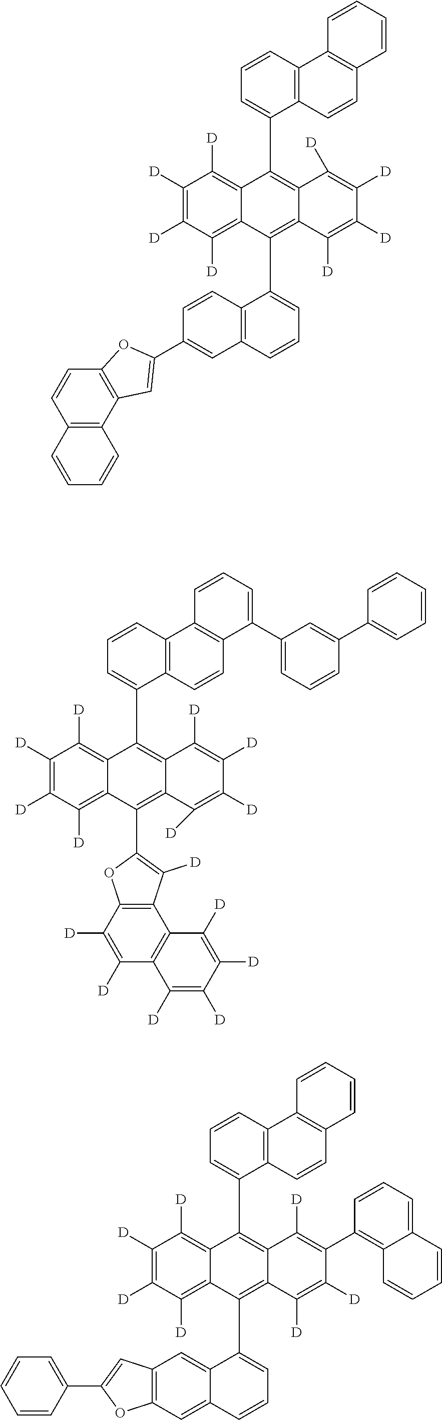 [SMILES: [2H]c1c([2H])c([2H])c2c(-c3cccc4c3ccc3ccccc34)c3c([2H])c([2H])c([2H])c([2H])c3c(-c3cccc4cc(-c5cc6c(ccc7ccccc76)o5)ccc34)c2c1[2H] ; [2H]c1c([2H])c([2H])c2c(-c3cccc4c3ccc3c(-c5cccc(-c6ccccc6)c5)cccc34)c3c([2H])c([2H])c([2H])c([2H])c3c(-c3oc4c([2H])c([2H])c5c([2H])c([2H])c([2H])c([2H])c5c4c3[2H])c2c1[2H] ; [2H]c1c([2H])c([2H])c2c(-c3cccc4c3ccc3ccccc34)c3c([2H])c(-c4cccc5ccccc45)c([2H])c([2H])c3c(-c3cccc4cc5oc(-c6ccccc6)cc5cc34)c2c1[2H]]

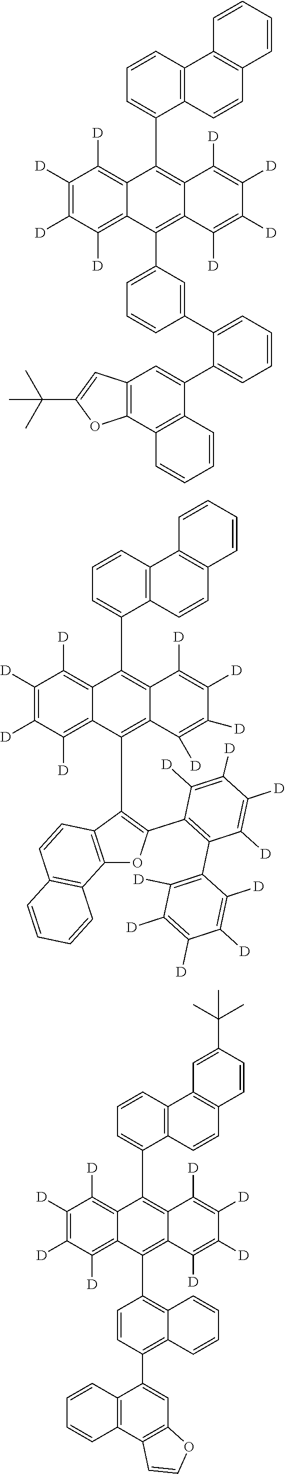

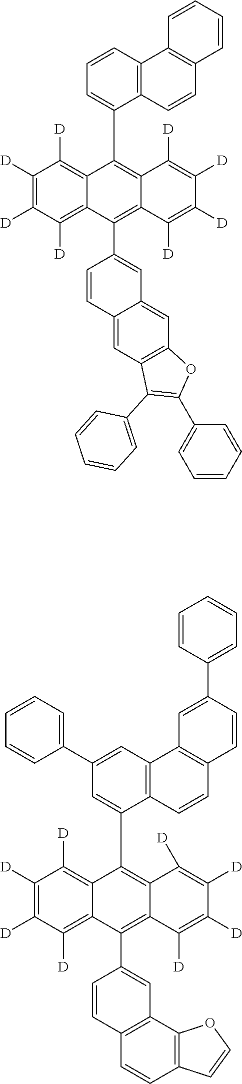 [SMILES: [2H]c1c([2H])c([2H])c2c(-c3cccc4c3ccc3ccccc34)c3c([2H])c([2H])c([2H])c([2H])c3c(-c3ccc4cc5c(-c6ccccc6)c(-c6ccccc6)oc5cc4c3)c2c1[2H] ; [2H]c1c([2H])c([2H])c2c(-c3cc(-c4ccccc4)cc4c3ccc3ccc(-c5ccccc5)cc34)c3c([2H])c([2H])c([2H])c([2H])c3c(-c3ccc4ccc5ccoc5c4c3)c2c1[2H]]

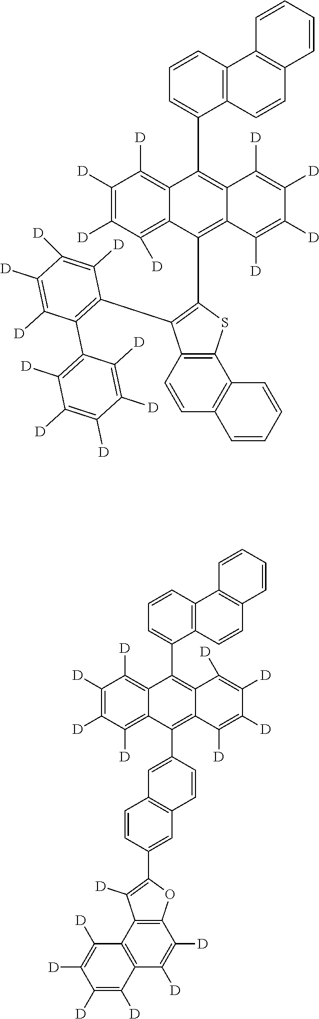 [SMILES: [2H]c1c([2H])c([2H])c(-c2c([2H])c([2H])c([2H])c([2H])c2-c2c(-c3c4c([2H])c([2H])c([2H])c([2H])c4c(-c4cccc5c4ccc4ccccc45)c4c([2H])c([2H])c([2H])c([2H])c34)sc3c2ccc2ccccc23)c([2H])c1[2H] ; [2H]c1c([2H])c([2H])c2c(-c3cccc4c3ccc3ccccc34)c3c([2H])c([2H])c([2H])c([2H])c3c(-c3ccc4cc(-c5oc6c([2H])c([2H])c7c([2H])c([2H])c([2H])c([2H])c7c6c5[2H])ccc4c3)c2c1[2H]]

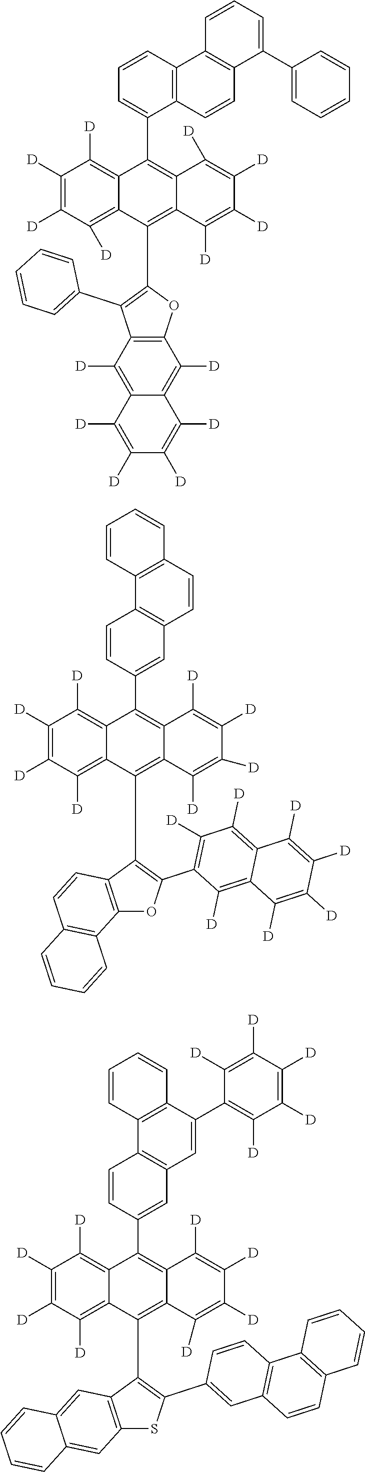

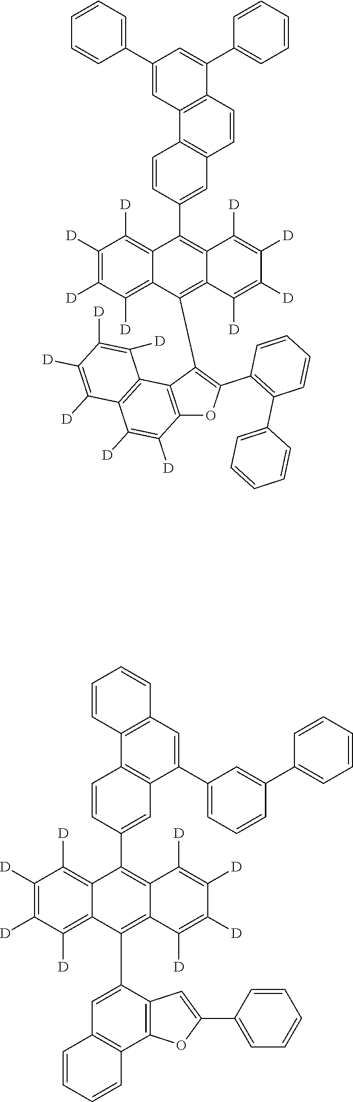 [SMILES: [2H]c1c([2H])c([2H])c2c(-c3c(-c4ccccc4-c4ccccc4)oc4c([2H])c([2H])c5c([2H])c([2H])c([2H])c([2H])c5c34)c3c([2H])c([2H])c([2H])c([2H])c3c(-c3ccc4c(ccc5c(-c6ccccc6)cc(-c6ccccc6)cc54)c3)c2c1[2H] ; [2H]c1c([2H])c([2H])c2c(-c3cc4ccccc4c4oc(-c5ccccc5)cc34)c3c([2H])c([2H])c([2H])c([2H])c3c(-c3ccc4c(c3)c(-c3cccc(-c5ccccc5)c3)cc3ccccc34)c2c1[2H]]

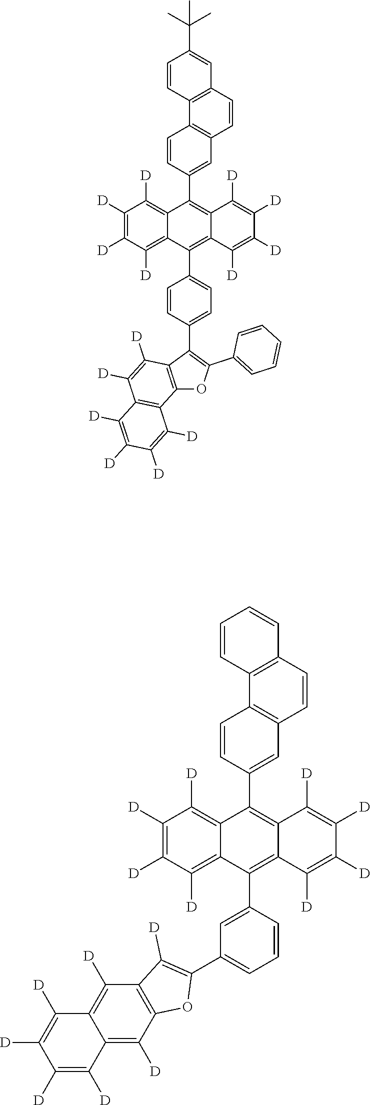 [SMILES: [2H]c1c([2H])c([2H])c2c(-c3ccc4c(ccc5cc(C(C)(C)C)ccc54)c3)c3c([2H])c([2H])c([2H])c([2H])c3c(-c3ccc(-c4c(-c5ccccc5)oc5c4c([2H])c([2H])c4c([2H])c([2H])c([2H])c([2H])c45)cc3)c2c1[2H] ; [2H]c1c([2H])c([2H])c2c([2H])c3c([2H])c(-c4cccc(-c5c6c([2H])c([2H])c([2H])c([2H])c6c(-c6ccc7c(ccc8ccccc87)c6)c6c([2H])c([2H])c([2H])c([2H])c56)c4)oc3c([2H])c2c1[2H]]

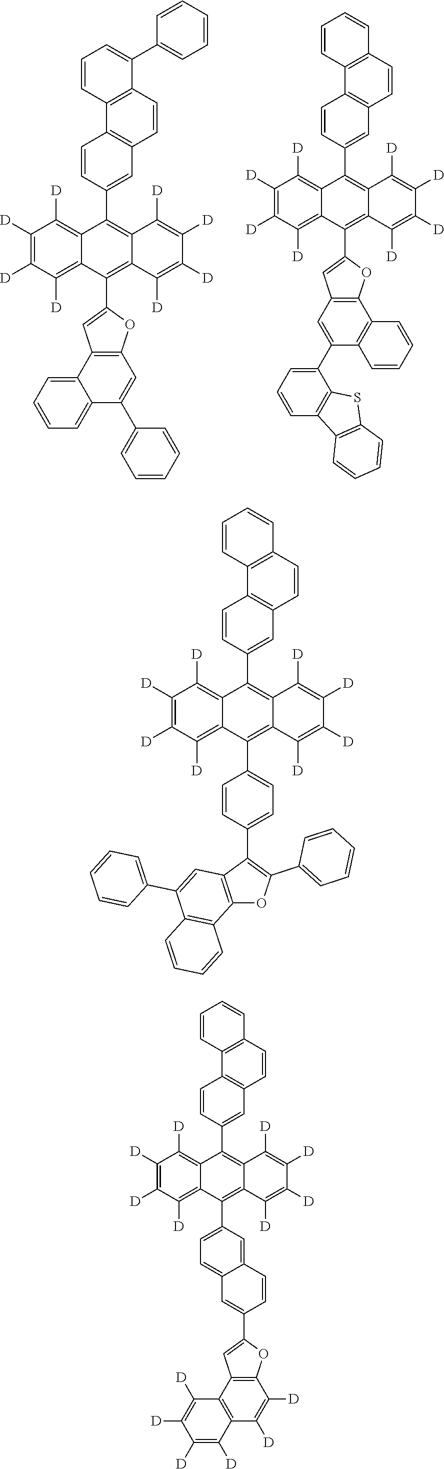

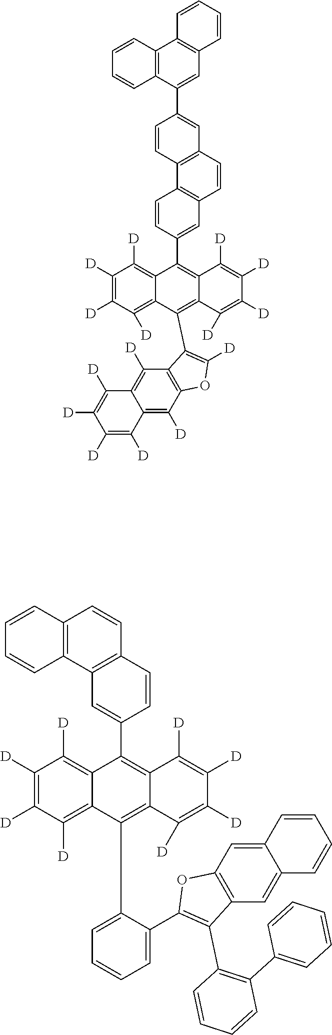 [SMILES: [2H]c1oc2c([2H])c3c([2H])c([2H])c([2H])c([2H])c3c([2H])c2c1-c1c2c([2H])c([2H])c([2H])c([2H])c2c(-c2ccc3c(ccc4cc(-c5cc6ccccc6c6ccccc56)ccc43)c2)c2c([2H])c([2H])c([2H])c([2H])c12 ; [2H]c1c([2H])c([2H])c2c(-c3ccccc3-c3oc4cc5ccccc5cc4c3-c3ccccc3-c3ccccc3)c3c([2H])c([2H])c([2H])c([2H])c3c(-c3ccc4ccc5ccccc5c4c3)c2c1[2H]]

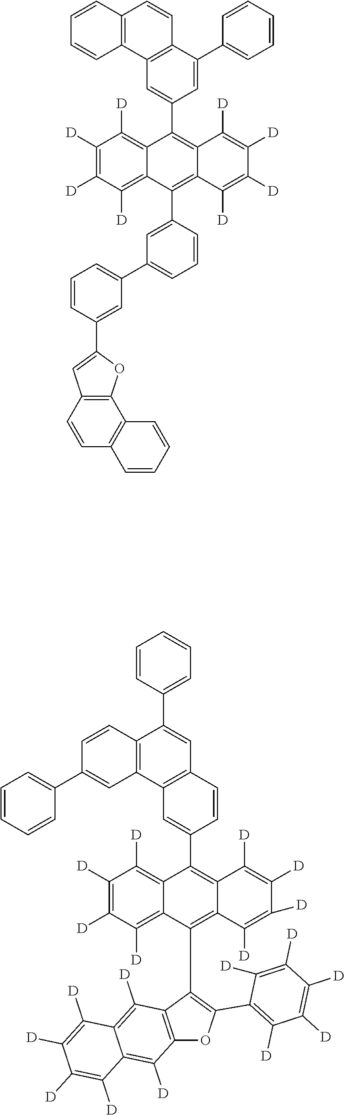 [SMILES: [2H]c1c([2H])c([2H])c2c(-c3cc(-c4ccccc4)c4ccc5ccccc5c4c3)c3c([2H])c([2H])c([2H])c([2H])c3c(-c3cccc(-c4cccc(-c5cc6ccc7ccccc7c6o5)c4)c3)c2c1[2H] ; [2H]c1c([2H])c([2H])c(-c2oc3c([2H])c4c([2H])c([2H])c([2H])c([2H])c4c([2H])c3c2-c2c3c([2H])c([2H])c([2H])c([2H])c3c(-c3ccc4cc(-c5ccccc5)c5ccc(-c6ccccc6)cc5c4c3)c3c([2H])c([2H])c([2H])c([2H])c23)c([2H])c1[2H]]

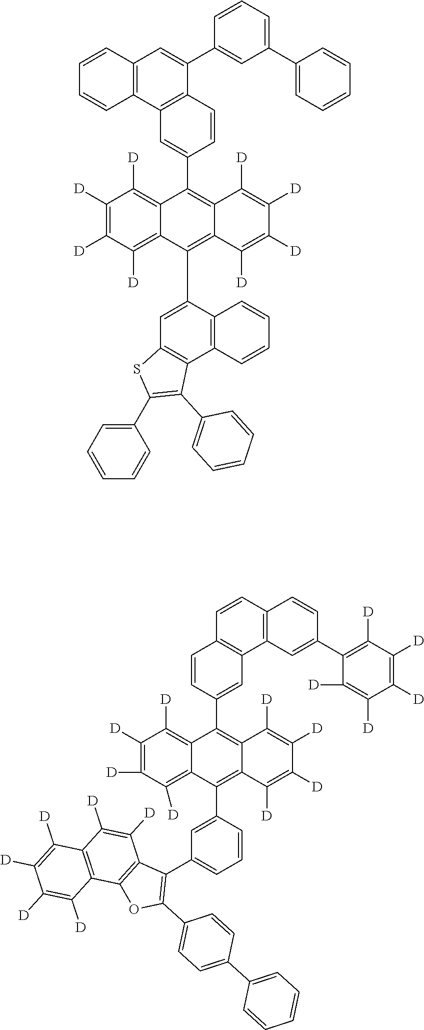 [SMILES: [2H]c1c([2H])c([2H])c2c(-c3cc4sc(-c5ccccc5)c(-c5ccccc5)c4c4ccccc34)c3c([2H])c([2H])c([2H])c([2H])c3c(-c3ccc4c(-c5cccc(-c6ccccc6)c5)cc5ccccc5c4c3)c2c1[2H] ; [2H]c1c([2H])c([2H])c(-c2ccc3ccc4ccc(-c5c6c([2H])c([2H])c([2H])c([2H])c6c(-c6cccc(-c7c(-c8ccc(-c9ccccc9)cc8)oc8c7c([2H])c([2H])c7c([2H])c([2H])c([2H])c([2H])c78)c6)c6c([2H])c([2H])c([2H])c([2H])c56)cc4c3c2)c([2H])c1[2H]]

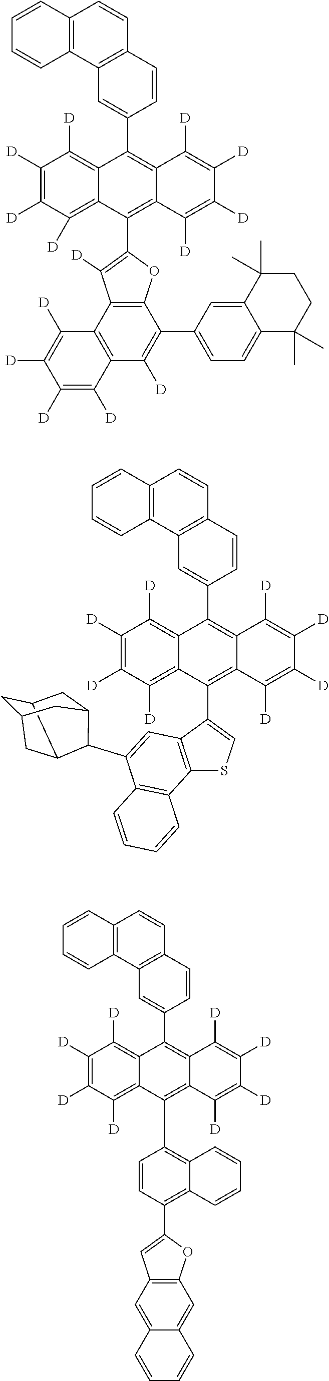 [SMILES: [2H]c1c([2H])c([2H])c2c(-c3oc4c(-c5ccc6c(c5)C(C)(C)CCC6(C)C)c([2H])c5c([2H])c([2H])c([2H])c([2H])c5c4c3[2H])c3c([2H])c([2H])c([2H])c([2H])c3c(-c3ccc4ccc5ccccc5c4c3)c2c1[2H] ; [2H]c1c([2H])c([2H])c2c(-c3csc4c3cc(C3C5CC6CC(C5)C3C6)c3ccccc34)c3c([2H])c([2H])c([2H])c([2H])c3c(-c3ccc4ccc5ccccc5c4c3)c2c1[2H] ; [2H]c1c([2H])c([2H])c2c(-c3ccc(-c4cc5cc6ccccc6cc5o4)c4ccccc34)c3c([2H])c([2H])c([2H])c([2H])c3c(-c3ccc4ccc5ccccc5c4c3)c2c1[2H]]

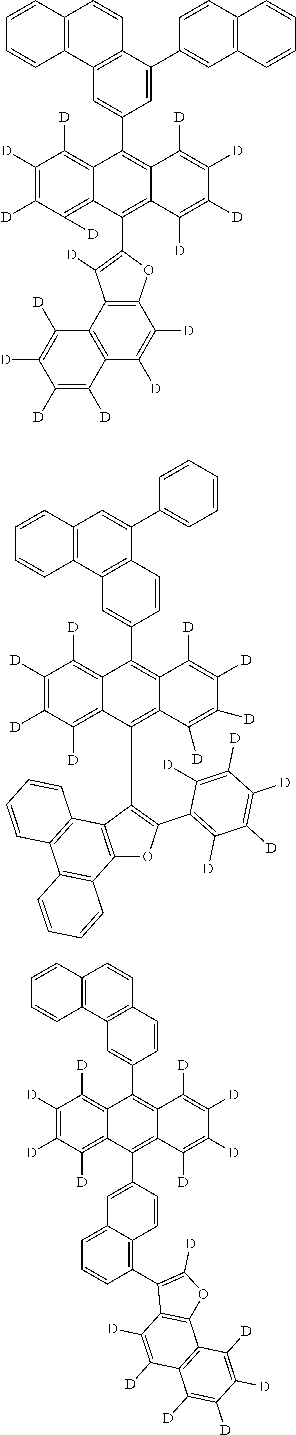

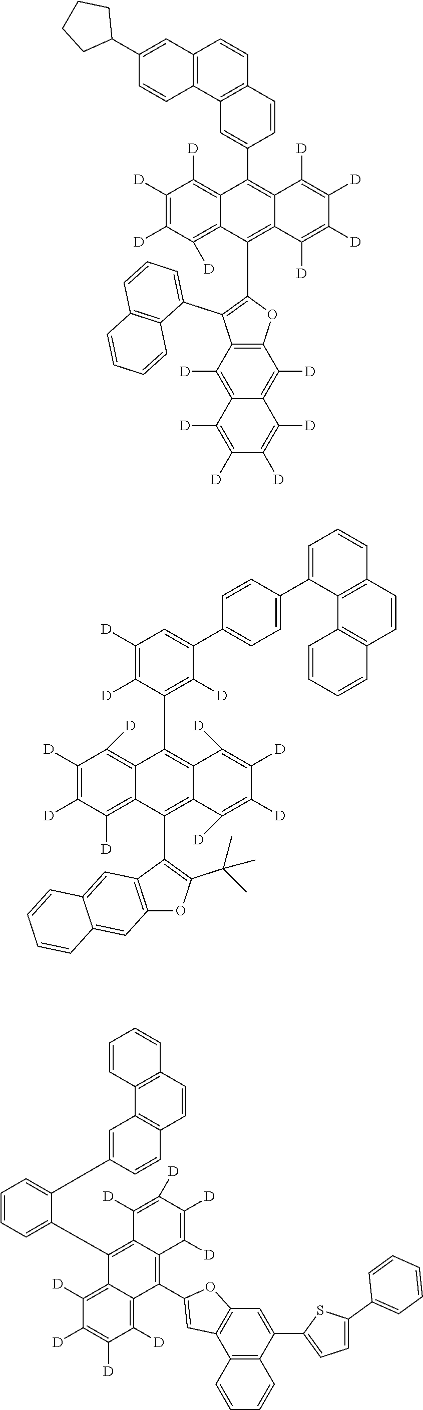 [SMILES: [2H]c1c([2H])c([2H])c2c([2H])c3c(-c4cccc5ccccc45)c(-c4c5c([2H])c([2H])c([2H])c([2H])c5c(-c5ccc6ccc7cc(C8CCCC8)ccc7c6c5)c5c([2H])c([2H])c([2H])c([2H])c45)oc3c([2H])c2c1[2H] ; [2H]c1cc(-c2ccc(-c3cccc4ccc5ccccc5c34)cc2)c([2H])c(-c2c3c([2H])c([2H])c([2H])c([2H])c3c(-c3c(C(C)(C)C)oc4cc5ccccc5cc34)c3c([2H])c([2H])c([2H])c([2H])c23)c1[2H] ; [2H]c1c([2H])c([2H])c2c(-c3ccccc3-c3ccc4ccc5ccccc5c4c3)c3c([2H])c([2H])c([2H])c([2H])c3c(-c3cc4c(cc(-c5ccc(-c6ccccc6)s5)c5ccccc54)o3)c2c1[2H]]

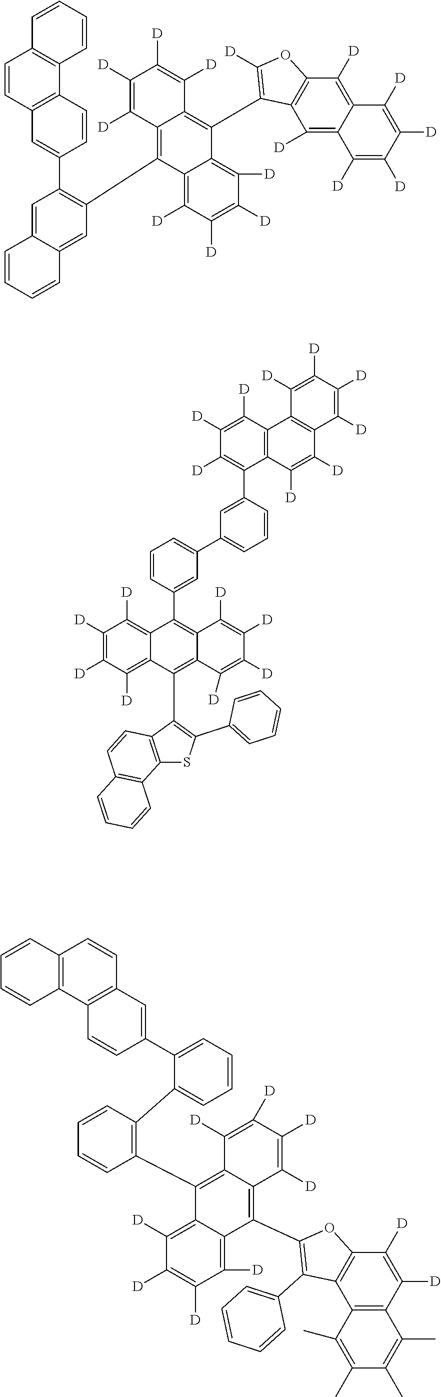

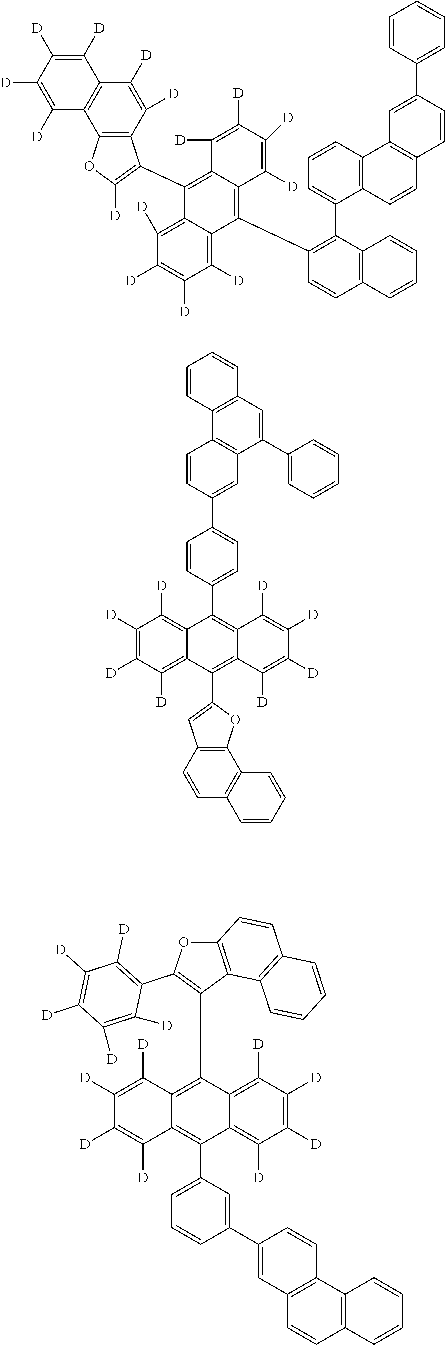 [SMILES: [2H]c1oc2c(c([2H])c([2H])c3c([2H])c([2H])c([2H])c([2H])c32)c1-c1c2c([2H])c([2H])c([2H])c([2H])c2c(-c2ccc3ccccc3c2-c2cccc3c2ccc2ccc(-c4ccccc4)cc23)c2c([2H])c([2H])c([2H])c([2H])c12 ; [2H]c1c([2H])c([2H])c2c(-c3cc4ccc5ccccc5c4o3)c3c([2H])c([2H])c([2H])c([2H])c3c(-c3ccc(-c4ccc5c(c4)c(-c4ccccc4)cc4ccccc45)cc3)c2c1[2H] ; [2H]c1c([2H])c([2H])c(-c2oc3ccc4ccccc4c3c2-c2c3c([2H])c([2H])c([2H])c([2H])c3c(-c3cccc(-c4ccc5c(ccc6ccccc65)c4)c3)c3c([2H])c([2H])c([2H])c([2H])c23)c([2H])c1[2H]]

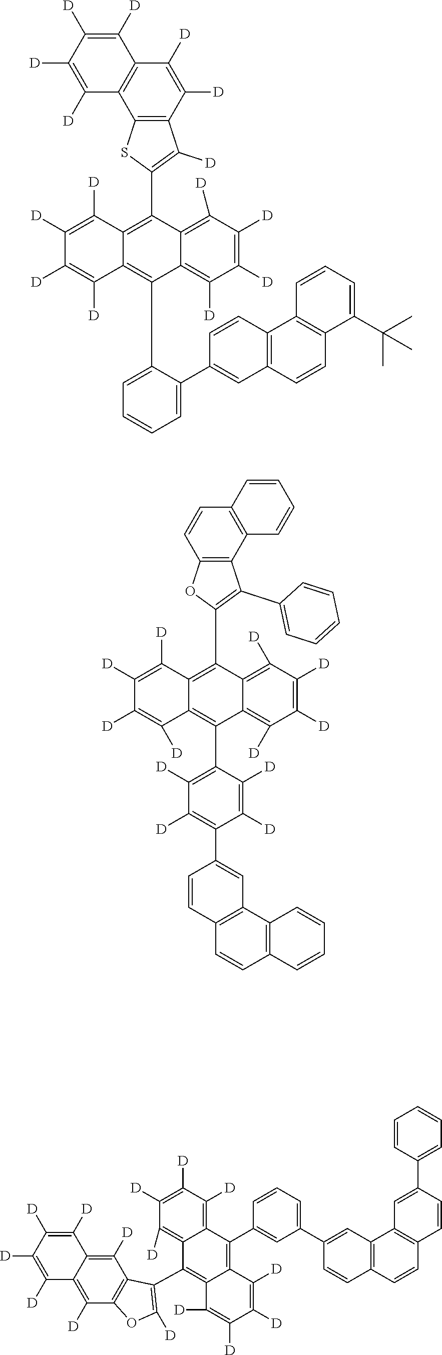

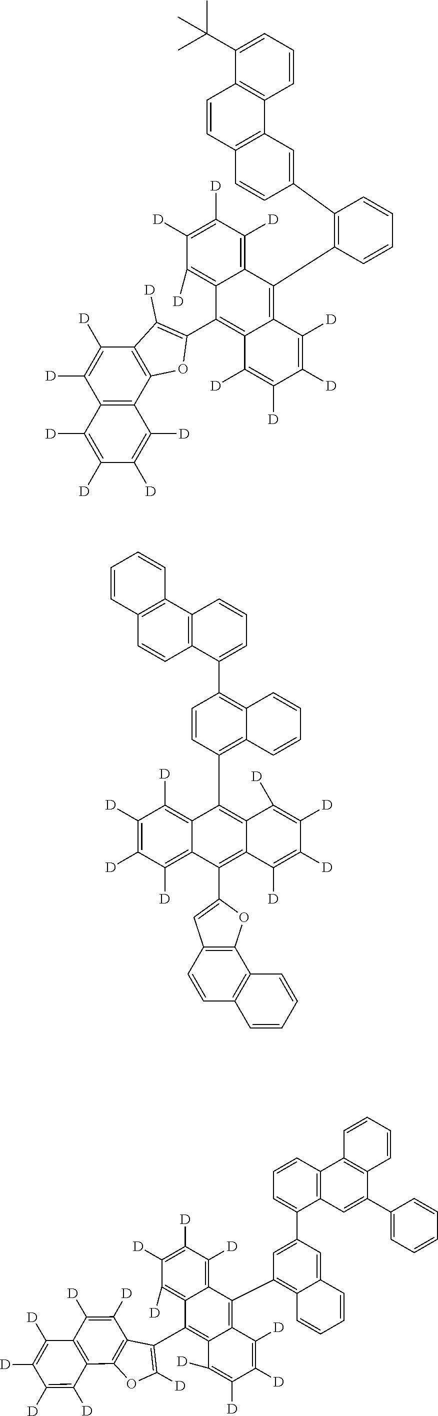 [SMILES: [2H]c1c([2H])c([2H])c2c(-c3oc4c(c3[2H])c([2H])c([2H])c3c([2H])c([2H])c([2H])c([2H])c34)c3c([2H])c([2H])c([2H])c([2H])c3c(-c3ccccc3-c3ccc4ccc5c(C(C)(C)C)cccc5c4c3)c2c1[2H] ; [2H]c1c([2H])c([2H])c2c(-c3ccc(-c4cccc5c4ccc4ccccc45)c4ccccc34)c3c([2H])c([2H])c([2H])c([2H])c3c(-c3cc4ccc5ccccc5c4o3)c2c1[2H] ; [2H]c1oc2c(c([2H])c([2H])c3c([2H])c([2H])c([2H])c([2H])c32)c1-c1c2c([2H])c([2H])c([2H])c([2H])c2c(-c2cc(-c3cccc4c3cc(-c3ccccc3)c3ccccc34)cc3ccccc23)c2c([2H])c([2H])c([2H])c([2H])c12]

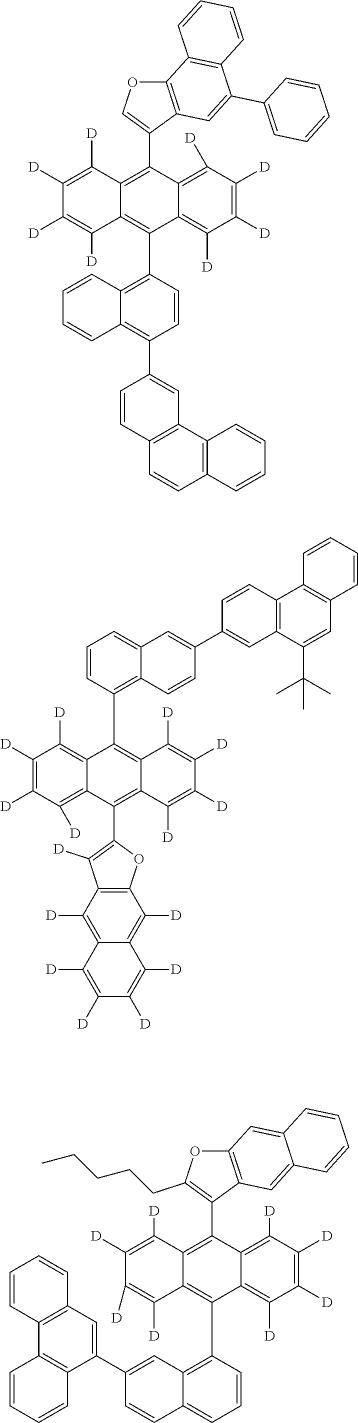 [SMILES: [2H]c1c([2H])c([2H])c2c(-c3coc4c3cc(-c3ccccc3)c3ccccc34)c3c([2H])c([2H])c([2H])c([2H])c3c(-c3ccc(-c4ccc5ccc6ccccc6c5c4)c4ccccc34)c2c1[2H] ; [2H]c1c([2H])c([2H])c2c([2H])c3c([2H])c(-c4c5c([2H])c([2H])c([2H])c([2H])c5c(-c5cccc6cc(-c7ccc8c(c7)c(C(C)(C)C)cc7ccccc78)ccc56)c5c([2H])c([2H])c([2H])c([2H])c45)oc3c([2H])c2c1[2H] ; [2H]c1c([2H])c([2H])c2c(-c3c(CCCCC)oc4cc5ccccc5cc34)c3c([2H])c([2H])c([2H])c([2H])c3c(-c3cccc4ccc(-c5cc6ccccc6c6ccccc56)cc34)c2c1[2H]]

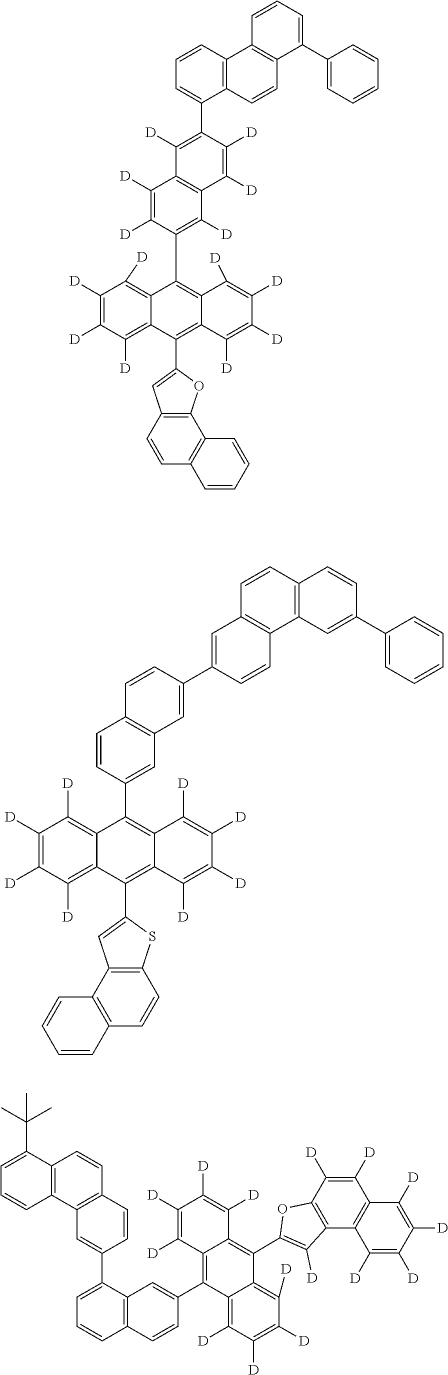 [SMILES: [2H]c1c(-c2c3c([2H])c([2H])c([2H])c([2H])c3c(-c3cc4ccc5ccccc5c4o3)c3c([2H])c([2H])c([2H])c([2H])c23)c([2H])c2c([2H])c([2H])c(-c3cccc4c3ccc3c(-c5ccccc5)cccc34)c([2H])c2c1[2H] ; [2H]c1c([2H])c([2H])c2c(-c3cc4c(ccc5ccccc54)s3)c3c([2H])c([2H])c([2H])c([2H])c3c(-c3ccc4ccc(-c5ccc6c(ccc7ccc(-c8ccccc8)cc76)c5)cc4c3)c2c1[2H] ; [2H]c1c([2H])c([2H])c2c(-c3oc4c([2H])c([2H])c5c([2H])c([2H])c([2H])c([2H])c5c4c3[2H])c3c([2H])c([2H])c([2H])c([2H])c3c(-c3ccc4cccc(-c5ccc6ccc7c(C(C)(C)C)cccc7c6c5)c4c3)c2c1[2H]]

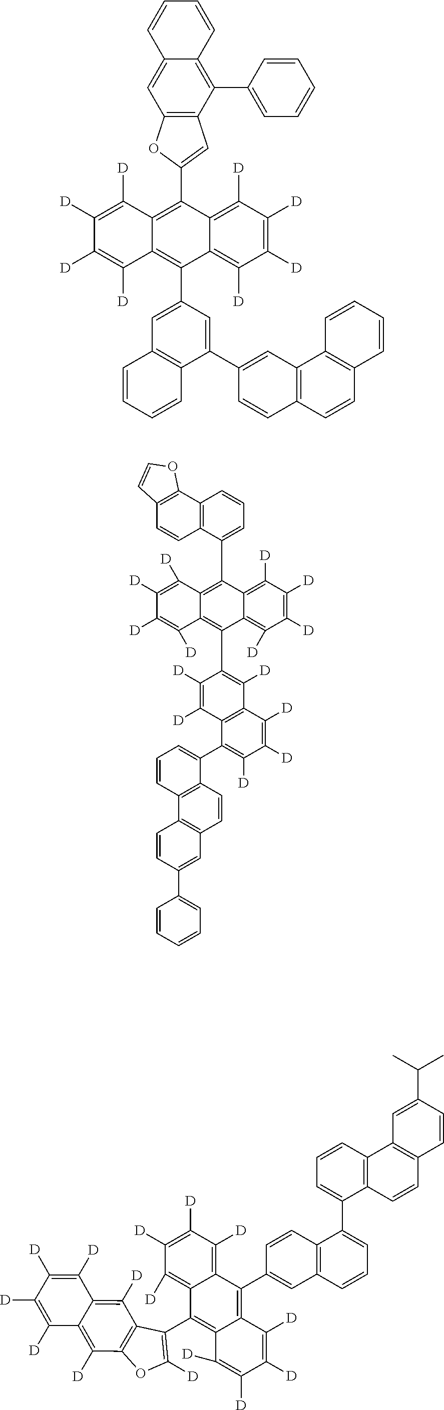 [SMILES: [2H]c1c([2H])c([2H])c2c(-c3cc4c(-c5ccccc5)c5ccccc5cc4o3)c3c([2H])c([2H])c([2H])c([2H])c3c(-c3cc(-c4ccc5ccc6ccccc6c5c4)c4ccccc4c3)c2c1[2H] ; [2H]c1c([2H])c(-c2cccc3c2ccc2cc(-c4ccccc4)ccc23)c2c([2H])c([2H])c(-c3c4c([2H])c([2H])c([2H])c([2H])c4c(-c4cccc5c4ccc4ccoc45)c4c([2H])c([2H])c([2H])c([2H])c34)c([2H])c2c1[2H] ; [2H]c1oc2c([2H])c3c([2H])c([2H])c([2H])c([2H])c3c([2H])c2c1-c1c2c([2H])c([2H])c([2H])c([2H])c2c(-c2ccc3c(-c4cccc5c4ccc4ccc(C(C)C)cc45)cccc3c2)c2c([2H])c([2H])c([2H])c([2H])c12]

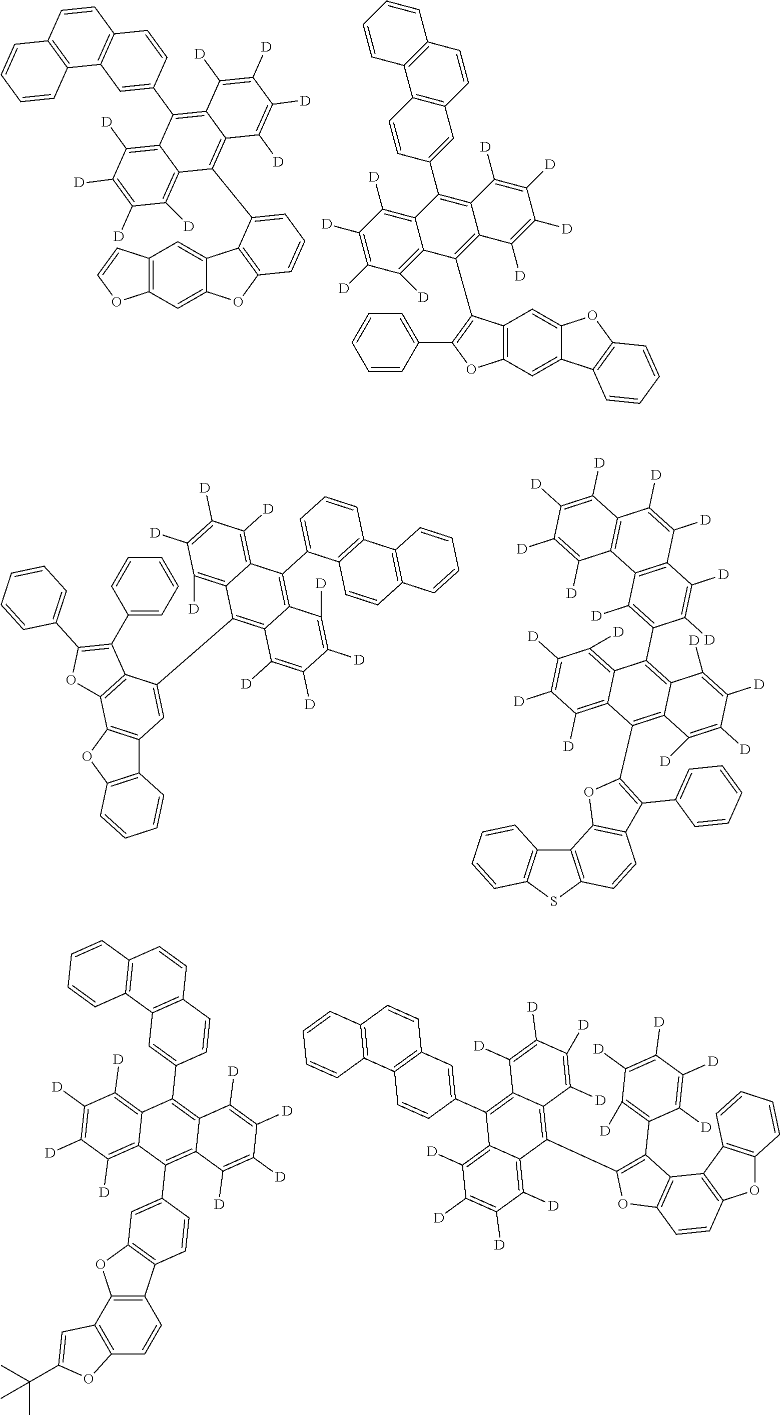

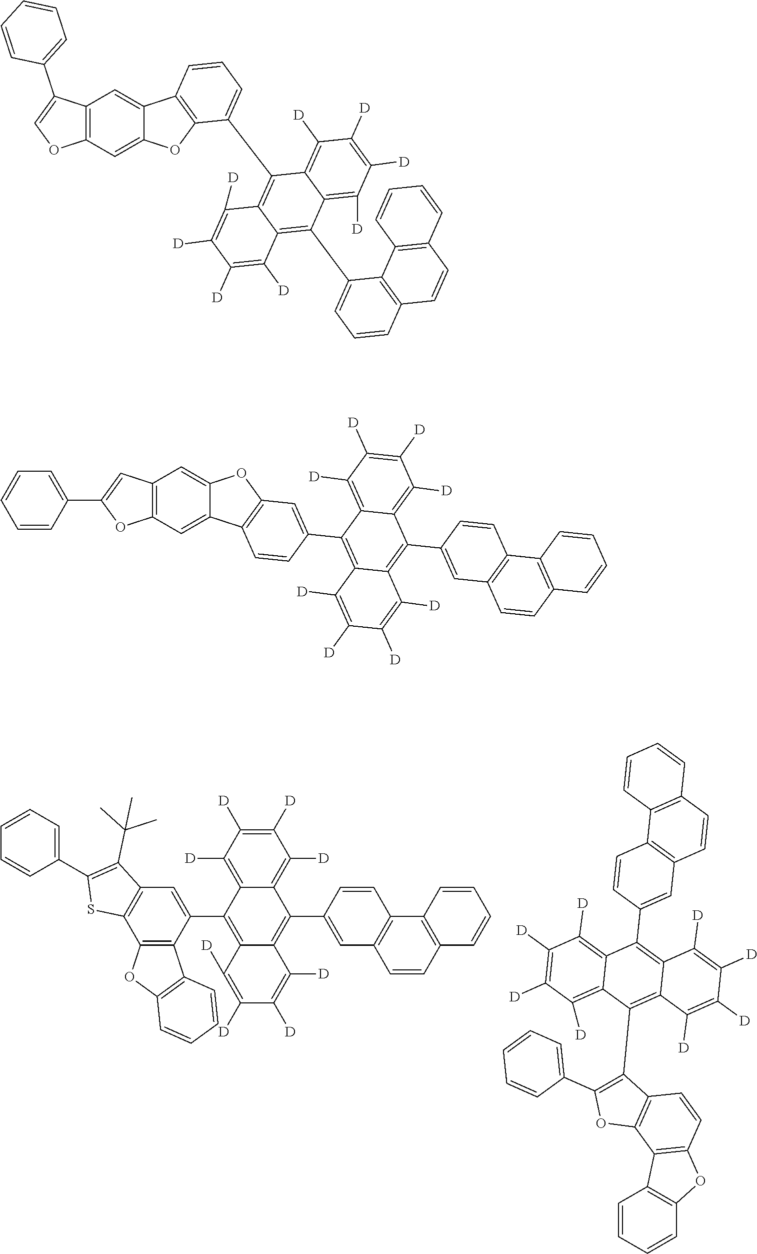

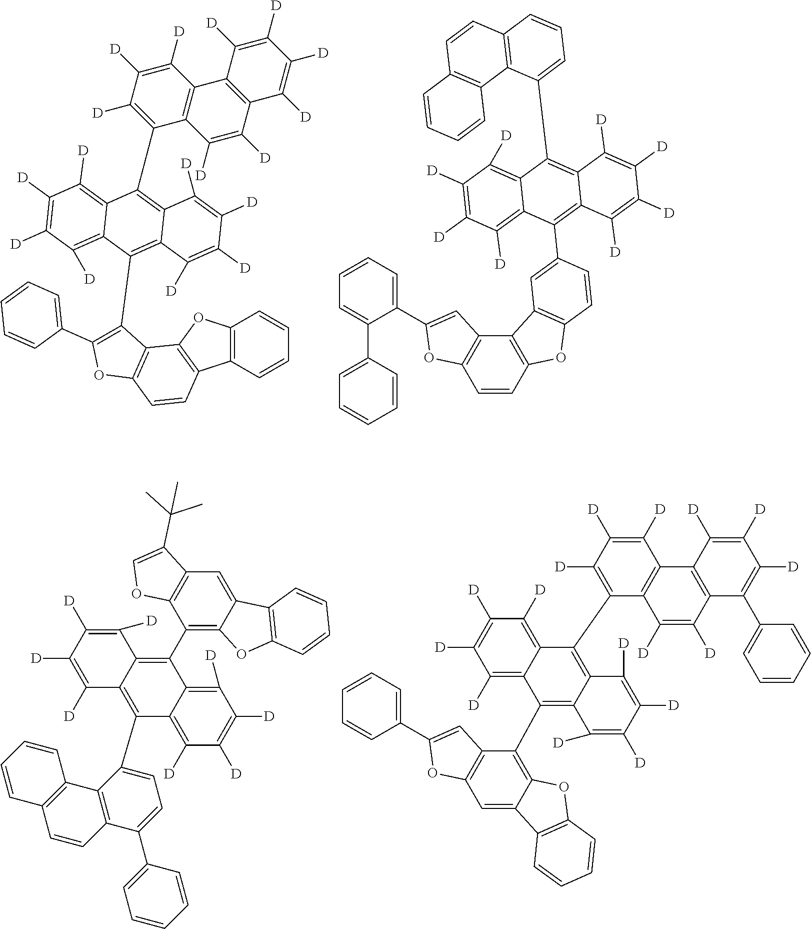

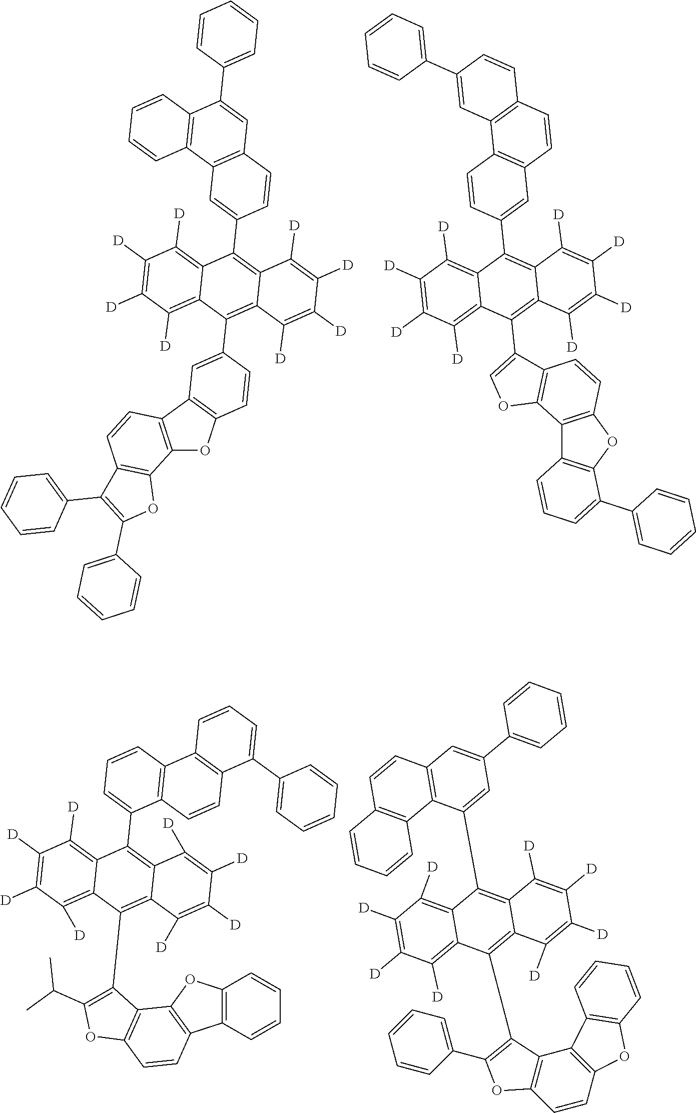

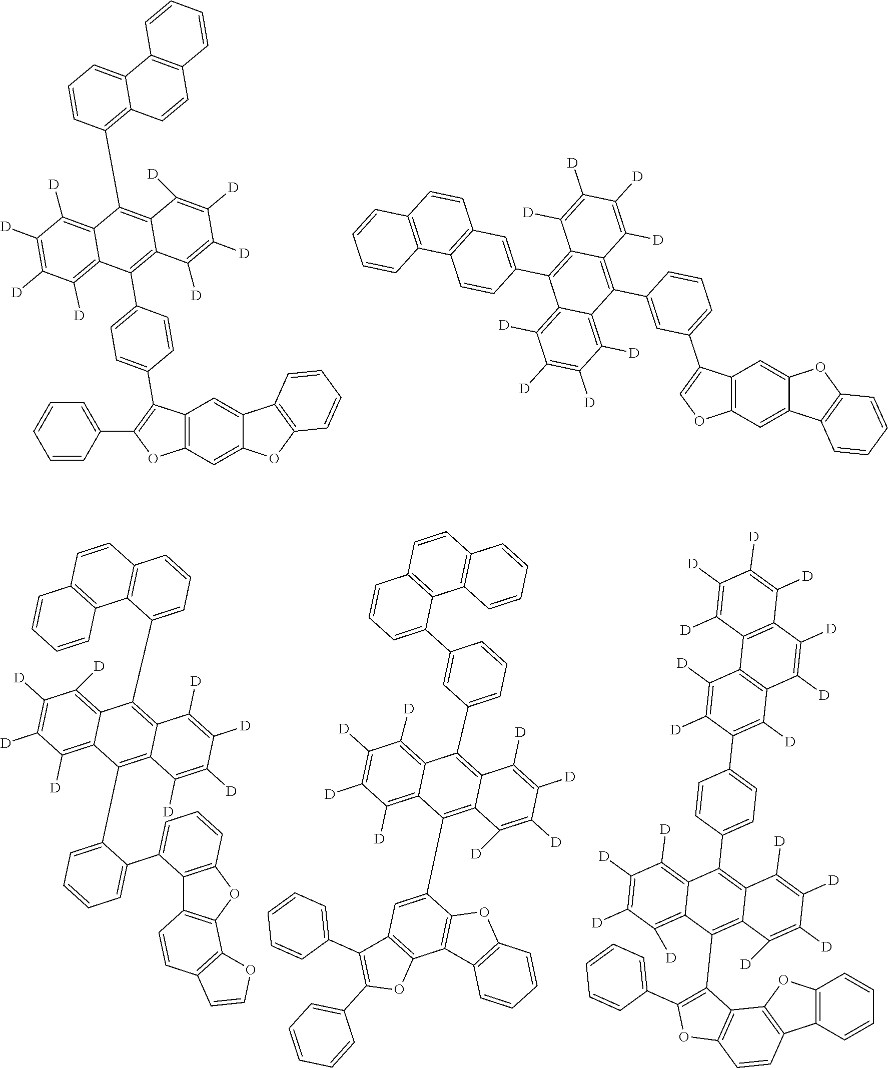

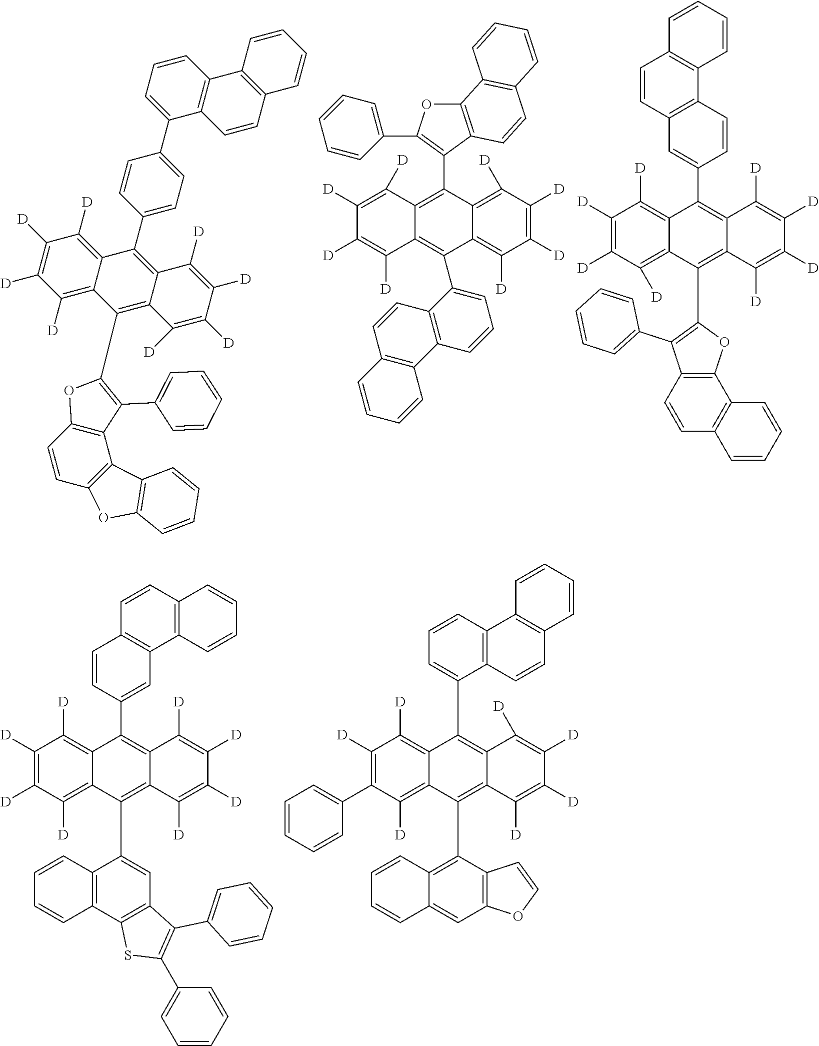

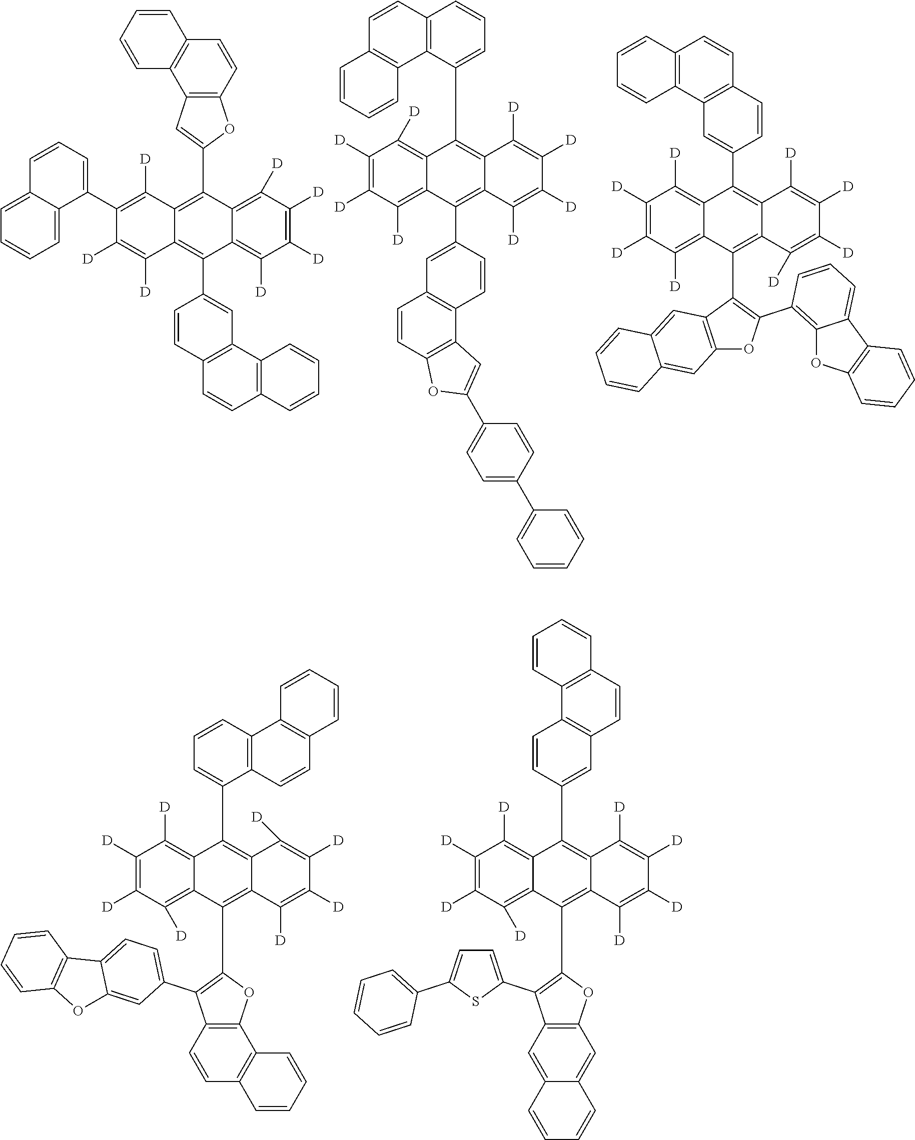

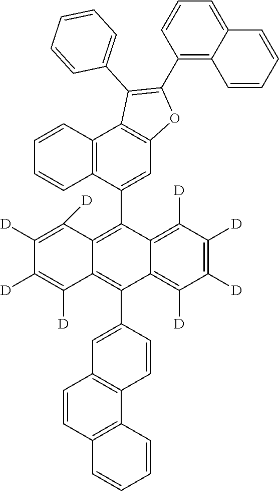 [SMILES: [2H]c1c([2H])c([2H])c2c(-c3cc4oc(-c5cccc6ccccc56)c(-c5ccccc5)c4c4ccccc34)c3c([2H])c([2H])c([2H])c([2H])c3c(-c3ccc4c(ccc5ccccc54)c3)c2c1[2H]]

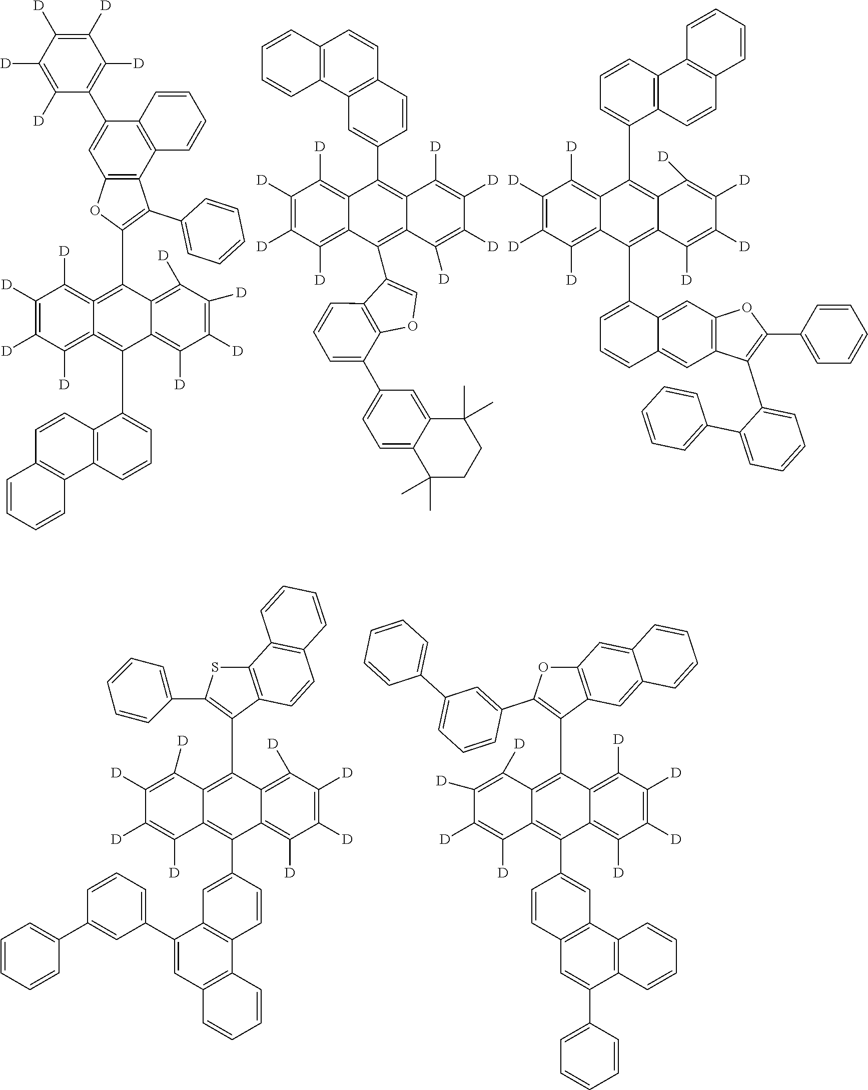

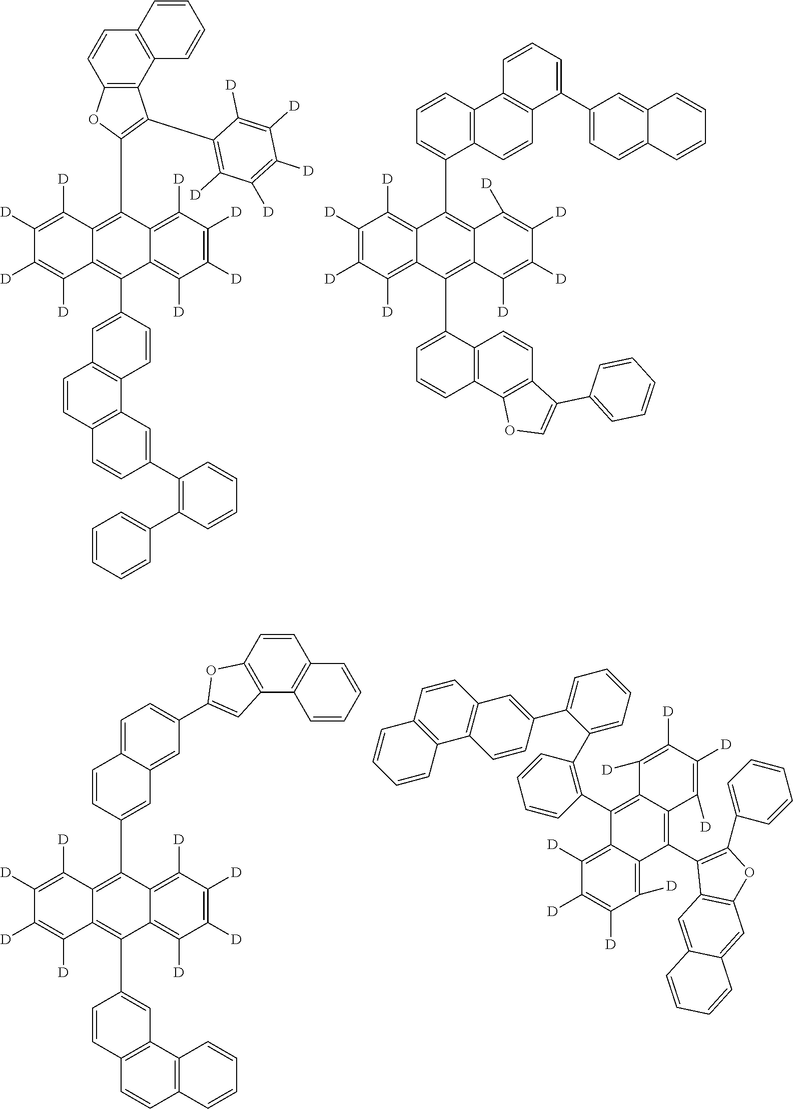

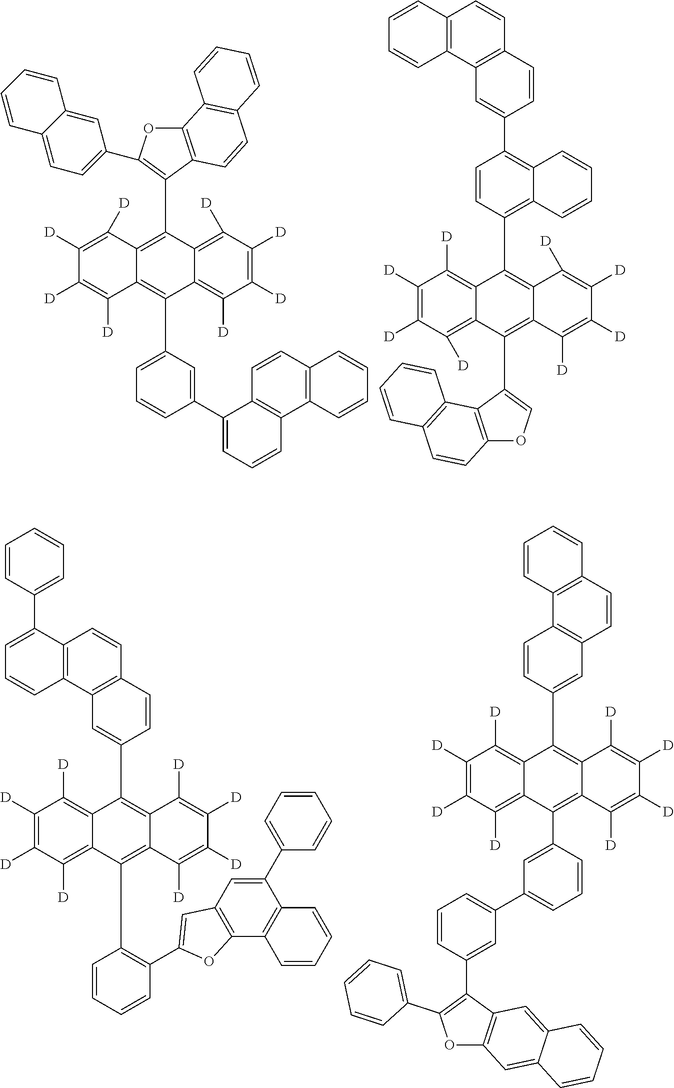

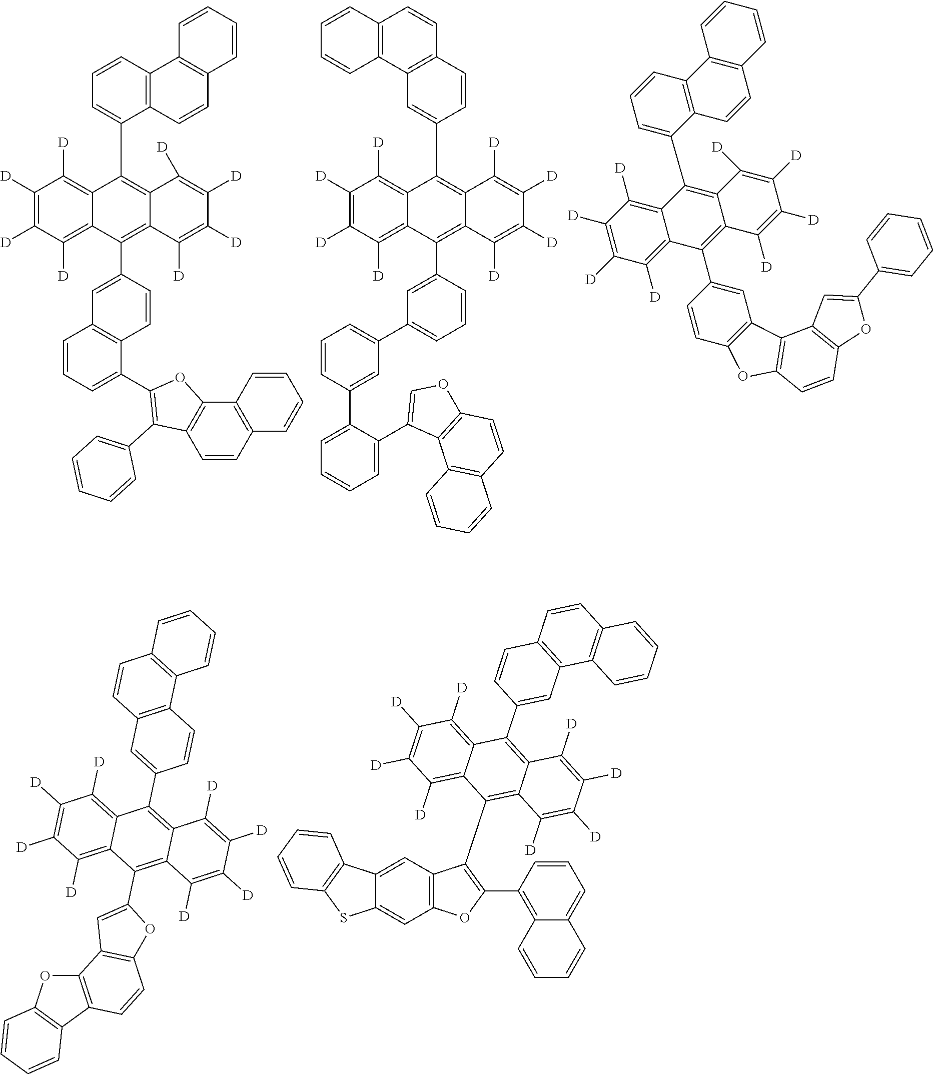

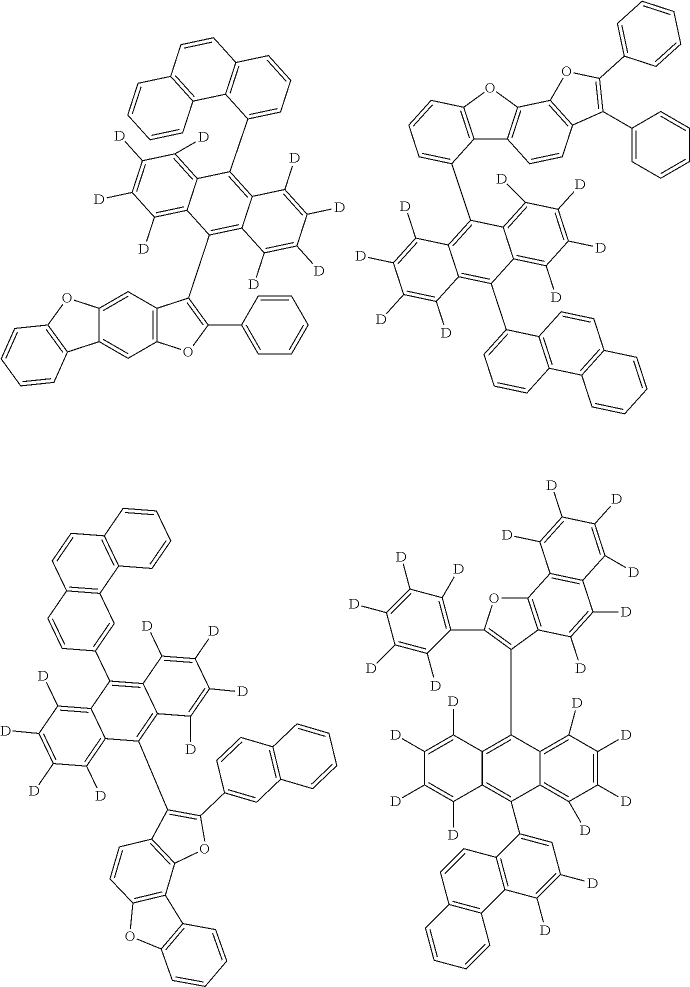

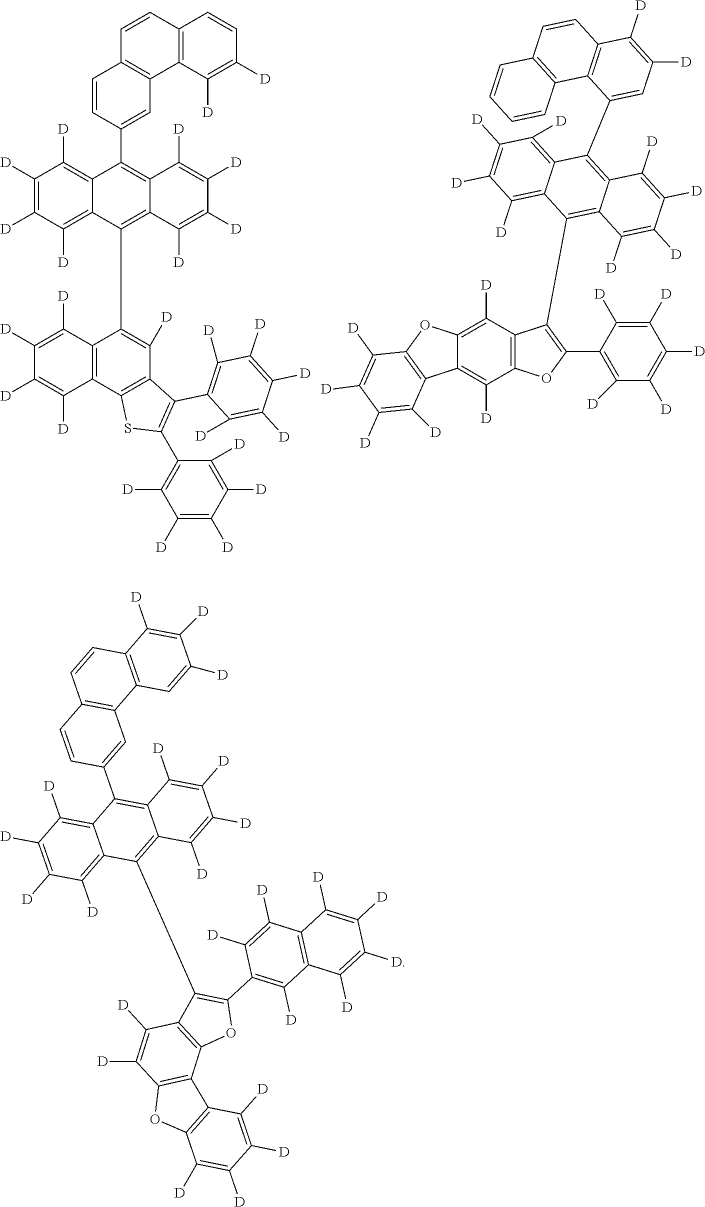

8. An organic light emitting device comprising:
an anode;
a cathode; and
an organic material layer having one or more layers provided between the anode and the cathode,
wherein one or more layers of the organic material layer comprise the organic compound according to claim 1.

9. The organic light emitting device of claim 8, wherein the organic material layer comprises a light emitting layer, and the light emitting layer comprises the organic compound.

10. The organic light emitting device of claim 9, wherein the light emitting layer comprises the organic compound as a host of the light emitting layer.

11. The organic light emitting device of claim 9, wherein the light emitting layer comprises the organic compound as a host and further comprises one host represented by the following Chemical Formula H-1 different from the organic compound: 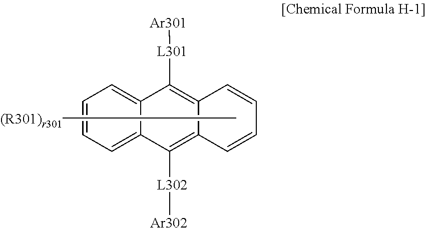
in Chemical Formula H-1,
L301 and L302 are the same as or different from each other, and are each independently a direct bond; a substituted or unsubstituted arylene group; or a substituted or unsubstituted heterocyclic group,
Ar301 and Ar302 are the same as or different from each other, and are each independently hydrogen; deuterium; a substituted or unsubstituted aryl group; or a substituted or unsubstituted heterocyclic group,
R301 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r301 is an integer from 1 to 7, and when r301 is 2 or higher, two or more R301's are the same as or different from each other.

12. The organic light emitting device of claim 10, wherein the light emitting layer comprises the organic compound as a host, and further comprises a dopant.
